(19) United States
(12) Patent Application Publication
(10) Pub. No.: US 2020/0239937 A1
(43) Pub. Date: Jul. 30, 2020

(54) BIOMARKERS FOR DIAGNOSING CONDITIONS

(71) Applicant: The Council of the Queensland Institute of Medical Research, Herston, Queensland (AU)

(72) Inventor: Jason LEE, Herston, Queensland (AU)

(21) Appl. No.: 16/487,798

(22) PCT Filed: Feb. 23, 2018
(86) PCT No.: PCT/AU2018/050156

§ 371 Date: Aug. 21, 2019

(30) Foreign Application Priority Data
Feb. 23, 2017 (AU) 2017900607

(51) Int. Cl. C12Q 1/6809; C12Q 1/6813; C12Q 1/6844; C12Q 1/6886

(52) U.S. Cl. CPC C12Q 1/6809; C12Q 1/6813; G01N 2800/7038; C12Q 1/6886; C12Q 2600/158; C12Q 1/6844

(57) ABSTRACT

Compositions, methods and apparatus for diagnosing and/or monitoring a hypoxic condition by measurement of a hypoxia-associated gene signature can be used for diagnosis including early diagnosis, monitoring, making treatment decisions, or management of subjects suspected of having a disease or condition that is associated with a hypoxic condition (e.g., a hypoxic condition). Nucleic acid and protein biomarkers can be used for specifically determining the likelihood of the presence or absence of a hypoxic condition in a subject.

### FIELD OF THE INVENTION

This application claims priority to Australian Provisional Application No. 2017900607 entitled “Biomarkers for diagnosing conditions” filed 23 Feb. 2017, the contents of which are incorporated herein by reference in their entirety.

This invention relates generally to compositions, methods and apparatus for diagnosing and/or monitoring a hypoxic condition by measurement of a hypoxia-associated gene signature. The invention can be used for diagnosis including early diagnosis, monitoring, making treatment decisions, or management of subjects suspected of having a disease or condition that is associated with a hypoxic condition (e.g., a hypoxic condition). More particularly, the present invention relates to nucleic acid and protein biomarkers that are useful for specifically determining the likelihood of the presence or absence of a hypoxic condition in a subject.

### BACKGROUND OF THE INVENTION

An important micro-environmental factor recognized to influence tumor behavior is hypoxia. In solid tumors, hypoxia occurs as a result of limitation in oxygen diffusion in vascular primary tumors or their metastases. Hypoxia in solid tumors is known to increase the aggressiveness of cancer cells by enhancing proliferative and metastatic potential (Harris, 2002; Semenza, 2003). Persistent hypoxia significantly reduces the efficacy of radiation and chemotherapy and leads to poor outcomes (Vaupel, 2004; Bache, et al., 2008). This is mainly due to the increase in pro-survival genes that suppress apoptosis (Erler et al., 2004), enhance tumor angiogenesis (Semenza, 2000) and the epithelial-to-mesenchymal transition (EMT) (Hill, et al., 2009). Clinical studies have also demonstrated that tumor hypoxia is strongly associated with an enhanced invasiveness (Pennacchietti et al., 2003) and a higher risk to develop metastasis (Chang et al., 2011), mainly considered to be due to the silencing of cell adhesion molecules (CAMs) under this condition (McGary et al., 2002; Lee et al., 2015; Scully et al., 2012).

Much of the tumor hypoxia research has been centered on examining the transcriptional targets of hypoxia inducible factors (HIFs). HIF-a is a heterodimeric transcription factor that is comprised of an oxygen-regulated a subunit (HIF-1a or HIF-2a) and a constitutively expressed β subunit (HIF-1β) (Ema et al., 1997; Semeza and Wang, 1992). HIF-1α is an oxygen-responsive transcription factor that mediates adaptation to hypoxia (Semenza, 2003; Dewhirst et al., 2008; Poon et al., 2009). In normal oxygen tension, HIF-a is hydroxylated on at least one of the two proline residues by the prolyl-hydroxylase domain (PHDs) containing enzymes (Ivan et al., 2001; Jaakkola et al., 2001). Hydroxylated HIF-a is then recognised by the tumor suppressor von Hippel Lindau protein (pVHL) and subsequently ubiquitinated for degradation by the proteasome (Maxwell et al., 1999; Ohh et al., 2000). As PHDs require oxygen for their enzymatic activity, under low oxygen concentrations, PHD-mediated hydroxylation is inhibited and HIF-a can then translocate to the nucleus, leading to specific target gene expression through binding of HIF-113 to a hypoxia response element (HRE, recognised by the motif RCGTG in which R is either A or G).

HIF-a activates metabolic and angiogenic genes that allow adaptation to hypoxic condition including Glut-1 and VEGFA (refs). HIF-a is also responsible for gene repression by activating transcriptional repressors such as DEC1 and DEC2 (Yun et al., 2002; Chakrabarti et al., 2004; Ivanov et al., 2007). While some genes are known to be transcriptionally downregulated by the recruitment of these specific repressors, it is increasingly evident that hypoxia-mediated gene repression also occurs independent of these repressive transcription factors. Therefore, not all genes are regulated by HIF-a, suggesting that some HIF-independent pathways may be involved in controlling gene expression in hypoxia.

G9a or euchromatic histone-lysine methyltransferase 2 (EHMT2) is one of a larger family of enzymes that can methylate histone H3 lysine 9 (H3K9) from an unmodified state to a dimethylated state (H3K9me2). Dimethylation of H3K9 is correlated with gene repression and is used as a marker of genes silenced epigenetically (Tachibana et al., 2005). G9a is frequently over-expressed in several tumor types and its depletion in cancer cells reduces tumor growth and metastasis suggesting that G9a participates in oncogenic and metastatic potential (Chen et al., 2010; Dong et al., 2012; Liu et al., 2015; Zhong et al., 2015; Hua et al., 2014; Wozniak et al., 2007). It has been shown that G9a protein accumulation occurs in hypoxic condition without altering the level of G9a transcript (Chen et al., 2006). However, the mechanism by which G9a exerts its activity on its environment is not well understood and therefore, the ability to use the identification and/or detection of G9a polypeptide in clinical diagnosis has not been possible.

### SUMMARY OF THE INVENTION

The present invention arises from the determination that certain host response peripheral blood RNA transcripts (RNA markers) are commonly, specifically and differentially expressed and regulated by G9a, particularly in hypoxic conditions. Such RNA transcripts (biomarkers) are useful for diagnosis at an early stage of a disease or condition and over the course of the disease or condition. These biomarkers are useful therefore in early diagnosis, diagnosis, monitoring, prognosis and determination of severity of a G9a-associated disease or condition. In one example, the disease or condition is associated with a hypoxic condition. In particular, based on the demonstrated specificity to hypoxia, such biomarkers are useful in determining the etiology of a disease or condition when caused by a hypoxic condition.

Based on this determination, the present inventors have developed various methods, apparatus, compositions, and kits, which take advantage of differentially expressed biomarkers, including ratios thereof (derived biomarkers), to determine the presence, absence or degree of G9a-associated disease or condition. in subjects presenting with clinical signs of the G9a-associated disease or condition. In certain embodiments, these methods, apparatus, compositions, and kits represent a significant advance over prior art processes and products, which have not been able to: 1) distinguish from other diseases or conditions that are not associated with aberrant G9a, (including other cancers); and/or 2) determine the contribution of a G9a overexpression (if any) to the presenting clinical signs and pathology of the disease or condition.

Accordingly, the present invention provides methods for determining an indicator used in assessing a likelihood of the presence or absence of a hypoxic condition (e.g., a hypoxic cancer) in a subject, the method comprising, consisting or consisting essentially of: (1) determining a biomarker value that is measured or derived for at least one hypoxia biomarker (e.g., 1, 2, 3, 4, 5, 6, 7, 8, 9 or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; and (2) determining the indicator using the biomarker value(s), wherein the indicator is at least partially indicative of the likelihood of the presence or absence of the hypoxic condition in the subject.

Non-limiting examples of nucleotide sequences for these hypoxia biomarkers are listed in SEQ ID NOs: 1-10 (see, Table 5). Non-limiting examples of amino acid sequences for these hypoxia biomarkers are listed in SEQ ID NOs: 202-211 (see, Table 6). In illustrative examples, an individual hypoxia biomarker is selected from the group consisting of: (a) a polynucleotide expression product comprising a nucleotide sequence that shares at least 70% (or at least 71% to at least 99% and all integer percentages in between) sequence identity with the sequence set forth in any one of SEQ ID NO: 1-10, or a complement thereof; (b) a polynucleotide expression product comprising a nucleotide sequence that encodes a polypeptide comprising the amino acid sequence set forth in any one of SEQ ID NO: 202-211; (c) a polynucleotide expression product comprising a nucleotide sequence that encodes a polypeptide that shares at least 70% (or at least 71% to at least 99% and all integer percentages in between) sequence similarity or identity with at least a portion of the sequence set forth in SEQ ID NO: 202-211; (d) a polynucleotide expression product comprising a nucleotide sequence that hybridizes to the sequence of (a), (b), (c) or a complement thereof, under medium or high stringency conditions; (e) a polypeptide expression product comprising the amino acid sequence set forth in any one of SEQ ID NO: 202-211; and (f) a polypeptide expression product comprising an amino acid sequence that shares at least 70% (or at least 71% to at least 99% and all integer percentages in between) sequence similarity or identity with the sequence set forth in any one of SEQ ID NO: 202-211.

Another aspect of the present invention provides methods for determining an indicator used in assessing malignancy of a tumor present in a subject, the method comprising, consisting, or consisting essentially of: (1) determining a biomarker value that is measured or derived for at least one hypoxia biomarker (e.g., 1, 2, 3, 4, 5, 6, 7, 8, 9 or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; and (2) determining the indicator using the biomarker value(s), wherein the indicator is at least partially indicative of the malignancy of the tumor.

One advantage of such methods, is that the hypoxia biomarkers may be detectable in the sample before the clinical signs of malignancy are observed. Thus, the present invention allows for the early detection of the likelihood of tumor malignancy, which is generally understood to correlate with increased survival rates of a subject.

Yet another aspect of the present invention provides methods for determining an indicator used in predicting a likelihood of cancer recurrence in a subject, the method comprising, consisting, or consisting essentially of: (1) determining a biomarker value that is measured or derived for at least one hypoxia biomarker (e.g., 1, 2, 3, 4, 5, 6, 7, 8, 9 or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; and (2) determining the indicator using the biomarker value(s), wherein the indicator is at least partially indicative of the likelihood of cancer recurring in the subject.

In another aspect, the present invention provides methods for reducing hypoxia in a subject, the method comprising, consisting or consisting essentially of: (1) determining a biomarker value that is measured or derived for at least one hypoxia biomarker (e.g., 1, 2, 3, 4, 5, 6, 7, 8, 9, or 10 hypoxia biomarkers) in a sample obtained from the subject, wherein the at least one hypoxia biomarker is selected from FGFR2, GATA2, CEACAM7, ARNTL, CD1C, KLRG1, OGN, MMP16, HHEX, and AGTR1; (2) determining an indicator using the biomarker value(s); and (3) administering an effective amount of a G9a antagonist to the subject on the basis that the indicator is at least partially indicative of the likelihood of the presence of hypoxia in the subject.

In a further aspect, the present invention provides methods for treating a hypoxic condition (e.g., a hypoxic cancer) in a subject, the method comprising, consisting, or consisting essentially of: (1) determining a biomarker value that is measured or derived for at least one hypoxia biomarker (e.g., 1, 2, 3, 4, 5, 6, 7, 8, 9 or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; (2) determining an indicator using the biomarker value(s); and (3) administering an effective amount of a G9a antagonist to the subject on the basis that the indicator is at least partially indicative of the likelihood of the presence of the hypoxic condition in the subject.

Yet another aspect of the present invention provides methods of reducing the malignancy of a hypoxic tumor in a subject, the method comprising, consisting, or consisting essentially of: (1) determining a biomarker value that is measured or derived for at least one hypoxia biomarker (e.g., 1, 2, 3, 4, 5, 6, 7, 8, 9, or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; (2) determining an indicator using the biomarker value(s); and (3) administering an effective amount of a G9a antagonist to the subject on the basis that the indicator is at least partially indicative of the likelihood that the tumor is hypoxic.

In another aspect, the present invention provides methods for treating a hypoxic tumor in a subject, the method comprising, consisting, or consisting essentially of: (1) determining a biomarker value that is measured or derived for at least one hypoxia biomarker (e.g., 1, 2, 3, 4, 5, 6, 7, 8, 9, or 10 hypoxia biomarkers) in sample obtained from the subject, wherein the at least one hypoxia biomarker is selected from ARNTL, CD1C, HHEX, KLRG1, MMP16, FGFR2, GATA2, CEACAM7, OGN, and AGTR1; and (2) determining an indicator using the biomarker value(s); and (3) administering an effective amount of a G9a antagonist to the subject on the basis that the indicator is at least partially indicative of the likelihood that the tumor is hypoxic.

Suitably, the sample is a biological sample, for example a biological sample comprising cancer or tumor cells.

In some embodiments, the subject is administered with an ancillary treatment.

For example, the subject may be administered with a G9a antagonist together with chemotherapy and/or radiotherapy treatment.

The at least one hypoxia biomarker is suitably selected from the group consisting of: (a) a polynucleotide expression product comprising a nucleotide sequence that shares at least 70% (or at least 71% to at least 99% and all integer percentages in between) sequence identity with the sequence set forth in any one of SEQ ID NO: 1-10, or a complement thereof; (b) a polynucleotide expression product comprising a nucleotide sequence that encodes a polypeptide comprising the amino acid sequence set forth in any one of SEQ ID NO: 202-211; (c) a polynucleotide expression product comprising a nucleotide sequence that encodes a polypeptide that shares at least 70% (or at least 71% to at least 99% and all integer percentages in between) sequence similarity or identity with at least a portion of the sequence set forth in SEQ ID NO: 202-211; (d) a polynucleotide expression product comprising a nucleotide sequence that hybridizes to the sequence of (a), (b), (c) or a complement thereof, under medium or high stringency conditions; (e) a polypeptide expression product comprising the amino acid sequence set forth in any one of SEQ ID NO: 202-211; and (f) a polypeptide expression product comprising an amino acid sequence that shares at least 70% (or at least 71% to at least 99% and all integer percentages in between) sequence similarity or identity with the sequence set forth in any one of SEQ ID NO: 202-211.

In some embodiments, the biomarker value is at least partially indicative of a concentration of the at least one hypoxia biomarker in the sample obtained from the subject. In some of the same embodiments and other embodiments, the biomarker value is at least partially indicated of the level of gene expression of the at least one hypoxia biomarker in the sample obtained from the subject. Suitably, the biomarker value includes the abundance of the biomarker.

In some embodiments, the level of the at least one hypoxia biomarker is reduced relative to the level of the biomarker that correlates with the presence of normal (i.e., non-hypoxic) conditions, and the indicator is thereby determined to be at least partially indicative of a hypoxia.

In some embodiments, the level of the at least one hypoxia biomarker is about the same as the level of the biomarker that correlates with the presence of normal (i.e., non-hypoxic) conditions, and the indicator is determined to be at least partially indicative of a normoxia.

In some embodiments, methods for determining an indicator used in assessing a likelihood of the presence or absence of a hypoxic condition (e.g., a hypoxic cancer) in a subject, the method comprising, consisting or consisting essentially of: (1) determining a biomarker value that is measured or derived for at least one group 1 hypoxia biomarker (e.g., 1, 2, 3, 4, 5, 6, 7, 8, 9 or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; (2) determining a biomarker value that is measured or derived for a group 2 hypoxia biomarker, wherein the group 2 hypoxia biomarker is G9a; and (3) determining the indicator using the biomarker values, wherein the indicator is at least partially indicative of the likelihood of the presence or absence of the hypoxic condition in the subject. Suitably, the methods further comprises applying a combining function to the at least one group 1 hypoxia biomarker value(s) and the group 2 hypoxia biomarker.

In some embodiments, wherein the indicator is a ratio of the biomarker values recorded on the group 1 hypoxia biomarker and the group 2 hypoxia biomarkers.

In the methods disclosed above and elsewhere herein, the biomarker value(s) is (are) measured using any suitable technique known in the art. For example, suitable measurements may be performed using any one or more of microscopy, flow cytometry, immunoassays, mass spectrometry, sequencing platforms, array and hybridization platforms, or a combination thereof.

In another aspect, the present invention provides compositions for determining an indicator used in assessing a likelihood of a subject having a hypoxic condition (e.g., hypoxic cancer). These compositions generally comprise, consist, or consist essentially of at least one cDNA and at least one oligonucleotide primer or probe that hybridizes to the cDNA, wherein the at least one cDNA is a selected from ARNTL, CD1C, HHEX, KLRG1, MMP16, FGFR2, GATA2, CEACAM7, OGN, and AGTR1. Suitably, the compositions comprise a population of cDNAs corresponding to mRNA derived from a cell or cell population. In some embodiments, the cell is a cell of suspected of a hypoxic condition, suitably a cancer or tumor cell. In some embodiments, the cell population is blood, suitably peripheral blood. In some embodiments, the at least one oligonucleotide primer or probe is hybridized to an individual one of the cDNAs. In any of the above embodiments, the composition may further comprise a labeled reagent for detecting the cDNA. In illustrative examples of this type, the labeled reagent is a labeled said at least one oligonucleotide primer or probe. In other embodiments, the labeled reagent is a labeled said cDNA. Suitably, the at least one oligonucleotide primer or probe is in a form other than a high density array. In non-limiting examples of these embodiments, the compositions comprise labeled reagents for detecting and/or quantifying no more than 4, 5, 6, 7, 8, 9, 10, 15, 20, 30, 40 or 50 different hypoxia biomarker cDNAs. In specific embodiments, the compositions comprise for a respective cDNA, (1) two oligonucleotide primers (e.g., nucleic acid amplification primers) that hybridize to opposite complementary strands of the cDNA, and (2) an oligonucleotide probe that hybridizes to the cDNA. In some embodiments, one or both of the oligonucleotide primers are labeled. In some embodiments, the oligonucleotide probe is labeled. In illustrative examples, the oligonucleotide primers are not labeled and the oligonucleotide probe is labeled. Suitably, in embodiments in which the oligonucleotide probe is labeled, the labeled oligonucleotide probe comprises a fluorophore. In representative examples of this type, the labeled oligonucleotide probe further comprises a quencher. In certain embodiments, different labeled oligonucleotide probes are included in the composition for hybridizing to different cDNAs, wherein individual oligonucleotide probes comprise detectably distinct labels (e.g. different fluorophores).

In still another aspect, the present invention provides complexes comprising, consisting, or consisting essentially of at least one cDNA and at least one oligonucleotide primer or probe that hybridizes to the cDNA, wherein the at least one cDNA is a selected from ARNTL, CD1C, HHEX, KLRG1, MMP16, FGFR2, GATA2, CEACAM7, OGN, and AGTR1. These compositions generally comprise, consist or consist essentially of at least one pair of cDNAs and at least one oligonucleotide primer or probe that hybridizes to an individual one of the cDNAs. Suitably, the compositions comprise a population of cDNAs corresponding to mRNA derived from a cell or cell population. In some embodiments, the cell is a cell of the immune system, suitably a leukocyte. In some embodiments, the cell population is blood, suitably peripheral blood. In some embodiments, the at least one oligonucleotide primer or probe is hybridized to an individual one of the cDNAs. In any of the above embodiments, the composition may further comprise a labeled reagent for detecting the cDNA. In illustrative examples of this type, the labeled reagent is a labeled said at least one oligonucleotide primer or probe. In other embodiments, the labeled reagent is a labeled said cDNA. Suitably, the at least one oligonucleotide primer or probe is in a form other than a high density array. In some embodiments, the cell is a cell of a cancer or tumor.

In other examples of these embodiments and other embodiments, the at least one oligonucleotide primer or probe is hybridized to an individual one of the cDNAs. Suitably, the composition or complex further comprises a labelled reagent for detecting the cDNAs. For example, in some embodiments, the labelled reagent is at least one oligonucleotide primer or probe. In some embodiments, the labelled reagent is a labelled said cDNA. In some of the same embodiments and other embodiments, the at least one oligonucleotide primer or probe is in a form other than a high density array.

In yet another aspect, the present invention provides a kit for determining an indicator indicative of the likelihood of hypoxia in a subject, the kit comprising, consisting, or consisting essentially of, (a) at least one reagent that allows quantification of a hypoxia biomarker;

and optionally (b) instructions for using the at least one reagent.

### BRIEF DESCRIPTION OF THE DRAWINGS

FIG. 1 is a photographical representation of G9a nuclear accumulation in hypoxic condition. (A) Immunoblotting analysis of G9a in nuclear extracts from MCF7 and MDA-MB-231 (MDA231) breast cancer cells exposed to normoxic and hypoxic conditions as indicated. HIF-1a levels were used as a marker of hypoxic response and Lamin A/C was used as a loading control. (B) Immunoblotting analysis of GLP and Suv39h1 in nuclear extracts from MCF7 cells exposed to normoxic and hypoxic conditions. (C) G9a transcript levels analyzed by qRT-PCR from RNA isolated from MCF7 cells exposed to hypoxic conditions as indicated. Results are expressed as relative mRNA levels compared to 0 hr (normoxia). (D) G9a immunoblotting was performed on nuclear extracts from MCF7 cells exposed to normoxic or hypoxic conditions for 9 hrs in the presence or absence of 20 μM proteasomal inhibitor, MG132. (E and F) Protein extracts from MCF7 cells transfected with His tagged-ubiquitin, exposed to normoxia or hypoxia (E) and DMOG treatment (1 mM) (F) as indicated were subjected to pull-down with Ni²⁺-NTA beads and immunoblotted for G9a. (G) G9a immunoblotting was performed on nuclear extracts from MCF7 and MDA231 cells treated with prolyl hydroxylase inhibitor (DMOG, 1 mM) for indicated times. (H) Immunoblotting of G9a from MCF7 and MDA231 cells in the presence or absence of 100 μM hypoxia-mimicking agent, Deferoxamine (DFA). Lamin A/C was used as loading control.

FIG. 2 is a photographical representation of the mechanism of G9a stabilisation under hypoxic stress. (A) MCF7 cells were transfected with the indicated expression plasmids in the presence of MG132 (20 μM), and immunoprecipitation was performed using anti-Flag antibody and immunoblotted using antibodies indicated. (B) G9a proline hydroxylation was determined in MCF7 cells overexpressing GFP-tagged PHD1, PHD2 or PHD3 in the presence of MG132 (20 μM). Immunoprecipitation of hydroxylated G9a was performed using anti-hydroxyproline antibody followed by immunoblotting with anti-Flag antibody. (C) Immunoprecipitation of proline hydroxylated G9a with anti-hydroxyproline antibody from MCF7 cells overexpressing Flag-tagged G9a either exposed to normoxia or hypoxia in the presence of MG132 (20 μM). (D and E) Interaction between G9a and pVHL was determined from MCF7 cells transfected with the indicated expression plasmids in normoxic or hypoxic conditions, and immunoprecipitation was performed using anti-Flag antibody (D) or cells treated with or without DMOG, immunoprecipitated with anti-HA (E) and immunoblotted using antibodies indicated. (F) Immunoblotting analysis of G9a in nuclear extracts from either RCC4 renal cell carcinoma cell line or overexpressing wild type pVHL exposed to normoxic and hypoxic conditions as indicated. (G) Immunoblotting analysis of G9a, in nuclear extracts from G9a^{-/-} MEFs reconstituted with G9a WT and P2A mutant exposed to normoxic and hypoxic conditions as indicated. (H) Immunoprecipitation of hydroxylated G9a with anti-hydroxyproline antibody from MCF7 cells overexpressing Flag-tagged G9a WT or P2A mutant either exposed to normoxia or hypoxia in the presence of MG132 (20 μM).

FIG. 3 shows graphical and schematic representations of the expression and prognostication of the hypoxia biomarker genes across breast cancer subtypes. (A) Hierarchical clustering of differentially expressed genes comparing fold change of hypoxia-responsive genes from MCF7 cells expressing shNS and shG9a. Upregulated and downregulated gene clusters are represented as red and green, respectively. (B) Diagram showing the strategy of cDNA microarray analysis and G9a-dependent gene selection process. (C) hypoxia biomarker gene signature; list of the 10 genes associated with relapse-free survival identified from FIG. 4 with a heat-map representing relative expression from the microarray analysis. (D) The average expression of the 10 G9a-suppressed genes was analyzed for association with relapse-free survival as a gene signature. The breast cancer cases in each of the three datasets (KM plotter, ROCK and TCGA) were allocated to one of four quartiles based on the G9a-suppressed gene signature and the survival of these patients were compared. The number of patients in each subgroup is shown in brackets and the hazard ratio (HR) and the log-rank P values for survival comparison between the quartile 1 group (bottom 25%) and the other groups is also shown in each panel. (G and H) Relapse-free survival analysis of breast cancer patients between tumors with the lowest expression (bottom 25%, quartile 1) to the rest of the tumors is shown using the G9a-suppressed gene signature in the different breast cancer subtypes from the KM plotter database. ER-positive and ER-negative (E) and Luminal A, Luminal B, HER2-enriched and Basal-like (F).

FIG. 4 is a schematic representation of the identification of G9a-associated genes. The hypoxia biomarker gene set from the microarray analysis was filtered for its inverse relationship with G9a and HIF target gene expression (left-hand circles in first step represent those genes that are inversely correlated to G9a; and right-hand circles represent those genes that are inversely correlated to hypoxia). Notably, 44 genes in each of the ER-positive and ER-negative groups identified were analyzed for commonality between the three datasets. 10 genes in each of the ER-positive and ER-negative groups were identified. Out of the 20 genes combined, 14 distinct genes were present (with 10 genes being associated with relapse-free survival).

FIG. 5 is a graphical representation of the molecular analysis of G9a inhibition in gene expression. (A) Quantitative RT-PCR analysis of the 10 hypoxia biomarker genes identified from FIG. 3 following UNC0642 treatment. Results are expressed as relative mRNA levels compared to vehicle treatment under normoxic (white boxes) or hypoxic (black boxes) conditions. (B) ChIP analysis of G9a, H3K9me2 and Pol II on AGTR1 and ARNTL promoters in MCF7 cells treated with 3 μM UNC0642 in normoxic or hypoxic conditions. (C) The shRNA-coupled ChIP assay on AGTR1 and ARNTL promoters in MCF7 cells in normoxia and hypoxia. Promoter occupancy by G9a, H3K9me2 and RNA polymerase II was analyzed. Values are expressed as mean±SEM. Statistical differences were determined by unpaired t-test (*P<0.05, **P<0.01), n=3.

FIG. 6 shows graphical and schematic representations of functional activity of G9a and G9a-dependent genes. (A) Top molecular and cellular functions altered in shG9a MCF7 cells in hypoxia include cellular development, growth and proliferation (p-value ranges: cell-to-cell signalling and interaction=8.10×10⁻³ to 2.07×10⁻⁶; cellular growth and proliferation=8.10×10⁻³ to 2.70×10⁻⁶; carbohydrate metabolism=8.10×10⁻³ to 4.22×10⁻⁵; cellular development=8.10×10⁻³ to 4.39×10⁻⁵; cellular function and maintenance=8.10×10⁻³ to 4.91×10⁻⁵). (B) Functional annotation network analysis as part of Ingenuity Pathway analysis revealed that downregulation of seven genes out of 10 genes in the hypoxia biomarker gene signature are predicted to inhibit organismal death (P=2.16×10⁻³) (i.e., upregulation of hypoxia biomarker gene signature promotes organismal death). Red shapes represent upregulated genes, green shapes represent downregulated genes, and dashed lines represent predicted inhibition. (C) Immunoblotting analysis of G9a in nuclear extracts from various breast epithelial cells as shown. Histone H3 levels were used as a loading control. (D) Cell survival was analyzed by performing MTT assay on cells examined in FIG. 6C following vehicle (black boxes) or G9a inhibitor (UNC0642 at 5 μM; clear boxes) treatment. (E) IncuCyte ZOOM time-lapse imaging analysis for MCF7 treated with various concentrations of UNC0642 as indicated. (D and E) Cell survival analyzed by performing MTT assay on MCF7 and MDA231 cells following vehicle or G9a inhibitor (UNC0642) treatment in normoxia (clear circles: vehicle; grey circles: UNC0642 1 μM; grey triangles: UNC0642 2 μM; and black squares: UNC0642 3 μM).

FIG. 7 is a photographical representation of the impact of G9a on cell motility. (A) Immunoblotting analysis of H3K9me2 in nuclear extracts from MCF7 cells following either vehicle or UNC0642 treatment for 6 hrs. Lamin A/C levels were used as a loading control. (B) Scratch wound assay for MCF7 cells, under both normoxic (21% 02) and hypoxic (1% 02) conditions. Results were evaluated by real-time imaging performed by the IncuCyte Zoom every 24 hours. Scale bars correspond to 700 μm, 10× magnification. (C) Scratch wound assay of MDA231 in normoxic (21% 02) and hypoxic (1% 02) conditions, grown in the presence or in the absence of G9a inhibitor. Scale bar 700 μm, 10× magnification. (D) Photomicrographs from a scratch wound assay of MCF7 cells expressing either shNS orshG9a, in hypoxia for the indicated times. Scale bars correspond to 1000 μm, 4× magnification.

FIG. 8 is a cartoon and graphical representation of the effect of G9a inhibition on tumor growth in vivo. (A) Diagram showing the design of the in vivo tumor growth study. (B) Groups of B6 wild-type (WT) mice (n=6-9) were subcutaneously injected with AT3 tumor (1×10⁶ cells) on day 0. Tumor-bearing mice were treated with 5 mg/kg UNC0642 intraperitoneally every two days. Tumor growth was measured using a digital caliper, and tumor volumes are represented as mean±SEM. Statistical differences in tumor volumes between vehicle and UNC0642-treated mice were determined by unpaired t-test (* P<0.05), n=3. (C) Tumor volume at end-point shown for vehicle and UNC0642-treated mice, represented as mean±SEM.

FIG. 9 is a graphical representation of the diagnosis and prognosis of a range of hypoxic cancers using the identified hypoxia biomarkers. (A) Kidney clear cell carcinoma patient dataset from The Cancer Genome Atlas (TCGA) were divided into quartiles, showing that patients with high expression of the hypoxia biomarkers are associated with a better survival outcome. (B) Lung adenocarcinoma patient dataset from Kaplan Meyer Plotter was divided into low and high groups, demonstrating that patients with high expression of the hypoxia biomarkers are associated with a better survival outcome. (C) Prognostic value of G9a assessed in melanoma. Patients grouped into a quartile based on the expression of a G9a transcript (Hazard ratio=1.773; P=0.0056), which quartile correlates to the overall survival. (D) Patients grouped into a quartile based on the expression of a G9a transcript (Hazard ratio=2.532; P=0.0106), which quartile correlates to the overall survival. (E) Prognostic value of G9a of outcome of patients with metastatic melanoma. The overall survival of patients stratified using G9a expression compared between melanoma patients and metastatic patients. (G) Patients grouped into quartiles based on average expression of a five-gene subset, namely ARNTL, CD1C, HHEX, KLRG1, and MMP16. The overall survival of patients associates with overall survival (OS) and relapse-free survival in melanoma.

### DETAILED DESCRIPTION OF THE INVENTION

### I. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” as used herein refers to the usual error range for the respective value readily known to the skilled person in this technical field. Reference to “about” a value or parameter herein includes (and describes) embodiments that are directed to that value or parameter per se.

As used herein, “and/or” refers to and encompasses any and all possible combinations of one or more of the associated listed items, as well as the lack of combinations when interpreted in the alternative (or).

The term “biomarker” broadly refers to any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a sample. “Derived from” as used in this context refers to a compound that, when detected, is indicative of a particular molecule being present in the sample. For example, detection of a particular cDNA can be indicative of the presence of a particular RNA transcript in the sample. As another example, detection of or binding to a particular antibody can be indicative of the presence of a particular antigen (e.g., protein) in the sample. Here, a discriminating molecule or fragment is a molecule or fragment that, when detected, indicates presence or abundance of an above-identified compound. A biomarker can, for example, be isolated from a sample, directly measured in a sample, or detected in or determined to be in a sample. A biomarker can, for example, be functional, partially functional, or non-functional. In specific embodiments, the “biomarkers” include “hypoxia biomarkers”, which are described in more detail below.

The term “biomarker value” refers to a value measured or derived for at least one corresponding biomarker of a subject and which is typically at least partially indicative of an abundance or concentration of a biomarker in a sample taken from the subject. Thus, the biomarker values could be measured biomarker values, which are values of biomarkers measured for the subject, or alternatively could be derived biomarker values, which are values that have been derived from one or more measured biomarker values, for example by applying a function to the one or more measured biomarker values. Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies and in one preferred example, the biomarker values relate to a level of activity or abundance of an expression product or other measurable molecule, quantified using a technique such as PCR, sequencing or the like. In this case, the biomarker values can be in the form of amplification amounts, or cycle times, which are a logarithmic representation of the concentration of the biomarker within a sample, as will be appreciated by persons skilled in the art and as will be described in more detail below.

The term “biomarker profile” refers to one or a plurality of one or more types of biomarkers (e.g., an mRNA molecule, a cDNA molecule and/or a protein, etc.), or an indication thereof, together with a feature, such as a measurable aspect (e.g., biomarker value) of the biomarker(s). A biomarker profile may comprise a single biomarker whose level, abundance or amount correlates with the presence or absence of a condition (e.g., hypoxia or normoxia). Alternatively, a biomarker profile may comprise at least two such biomarkers or indications thereof, where the biomarkers can be in the same or different classes, such as, for example, a nucleic acid and a polypeptide. Thus, a biomarker profile may comprise at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 or more biomarkers or indications thereof. In some embodiments, a biomarker profile comprises hundreds, or even thousands, of biomarkers or indications thereof. A biomarker profile can further comprise one or more controls or internal standards. In certain embodiments, the biomarker profile comprises at least one biomarker, or indication thereof, that serves as an internal standard. In other embodiments, a biomarker profile comprises an indication of one or more types of biomarkers. The term “indication” as used herein in this context merely refers to a situation where the biomarker profile contains symbols, data, abbreviations or other similar indicia for a biomarker, rather than the biomarker molecular entity itself. The term “biomarker profile” is also used herein to refer to a biomarker value or combination of at least two biomarker values, wherein individual biomarker values correspond to values of biomarkers that can be measured or derived from one or more subjects, which combination is characteristic of a discrete condition, stage of condition, subtype of condition or a prognosis for a discrete condition, stage of condition, subtype of condition. The term “profile biomarkers” is used to refer to a subset of the biomarkers that have been identified for use in a biomarker profile that can be used in performing a clinical assessment, such as to rule in or rule out a specific condition, different stages or severity of conditions, subtypes of different conditions or different prognoses. The number of profile biomarkers will vary, but is typically of the order of 10 or less.

The terms “complementary” and “complementarity” refer to polynucleotides (i.e., a sequence of nucleotides) related by the base-pairing rules. For example, the sequence “A-G-T”, is complementary to the sequence “T-C-A.” Complementarity may be “partial”, in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “correlating” refers to determining a relationship between one type of data with another or with a state.

As used herein, the terms “detectably distinct” and “detectably different” are used interchangeably herein to refer to a signal that is distinguishable or separable by a physical property either by observation or by instrumentation. For example, a fluorophore is readily distinguishable either by spectral characteristics or by fluorescence intensity, lifetime, polarization or photo-bleaching rate from another fluorophore in a sample, as well as from additional materials that are optionally present. In certain embodiments, the terms “detectably distinct” and “detectably different” refer to a set of labels (such as dyes, suitably organic dyes) that can be detected and distinguished simultaneously.

As used herein, the terms “diagnosis”, “diagnosing” and the like are used interchangeably herein to encompass determining the likelihood that a subject will develop a condition, or the existence or nature of a condition in a subject. These terms also encompass determining the severity of disease or episode of disease, as well as in the context of rational therapy, in which the diagnosis guides therapy, including initial selection of therapy, modification of therapy (e.g., adjustment of dose or dosage regimen), and the like. By “likelihood” is meant a measure of whether a subject with particular measured or derived biomarker values actually has a condition (or not) based on a given mathematical model. An increased likelihood for example may be relative or absolute and may be expressed qualitatively or quantitatively. For instance, an increased likelihood may be determined simply by determining the subject's measured or derived biomarker values for at least two hypoxia biomarkers and placing the subject in an “increased likelihood” category, based upon previous population studies. The term “likelihood” is also used interchangeably herein with the term “probability”. The term “risk” relates to the possibility or probability of a particular event occurring at some point in the future. “Risk stratification” refers to an arraying of known clinical risk factors to allow physicians to classify patients into a low, moderate, high or highest risk of developing a particular disease or condition.

“Fluorophore” as used herein to refer to a moiety that absorbs light energy at a defined excitation wavelength and emits light energy at a different defined wavelength. Examples of fluorescence labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red and Texas Red-X.

The term “gene”, as used herein, refers to a stretch of nucleic acid that codes for a polypeptide or for an RNA chain that has a function. While it is the exon region of a gene that is transcribed to form mRNA, the term “gene” also includes regulatory regions such as promoters and enhancers that govern expression of the exon region.

The term “high-density array” refers to a substrate or collection of substrates or surfaces bearing a plurality of array elements (e.g., discrete regions having particular moieties, e.g., proteins (e.g., antibodies), nucleic acids (e.g., oligonucleotide probes), etc., immobilized thereto), where the array elements are present at a density of about 100 elements/cm² or more, about 1,000 elements/cm² or more, about 10,000 elements/cm² or more, or about 100,000 elements/cm² or more. In specific embodiments, a “high-density array” is one that comprises a plurality of array elements for detecting about 100 or more different biomarkers, about 1,000 or more different biomarkers, about 10,000 or more different biomarkers, or about 100,000 or more different biomarkers. In representative example of these embodiments, a “high-density array” is one that comprises a plurality of array elements for detecting biomarkers of about 100 or more different genes, of about 1,000 or more different genes, of about 10,000 or more different genes, or of about 100,000 or more different genes. Generally, the elements of a high-density array are not labeled. The term “low-density array” refers to a substrate or collection of substrates or surfaces bearing a plurality of array elements (e.g., discrete regions having particular moieties, e.g., proteins (e.g., antibodies), nucleic acids (e.g., oligonucleotide probes), etc., immobilized thereto), where the array elements are present at a density of about 100 elements/cm² or less, about 50 elements/cm² or less, about 20 elements/cm² or less, or about 10 elements/cm² or less. In specific embodiments, a “low-density array” is one that comprises a plurality of array elements for detecting about 100 or less different biomarkers, about 50 or less different biomarkers, about 20 or less different biomarkers, or about 10 or less different biomarkers. In representative example of these embodiments, a “low-density array” is one that comprises a plurality of array elements for detecting biomarkers of about 100 or less different genes, of about 50 or less different genes, of about 20 or less different genes, or of about 10 or less different genes. Generally, the elements of a low-density array are not labeled.

As used herein, the term “hypoxia” refers to an environment in which the oxygen tension of tissue cells is abnormally low compared to that of normal tissue. Such an environment can occur when a tissue is compromised or blood flow. It appears commonly in intractable diseases, including cancer, ischemic stroke, and arthritis. In the case of cancer, as cancer tissue grows, it encounters a hypoxic environment because the inside of solid cancer does not receive oxygen from blood vessels. In conditions of in vitro cell culture, hypoxia may refer to an environment with at most about 5% O₂, preferably to a environment with about 1% O₂. In contrast, the term “normoxia” refers to an environment with an oxygen tension that corresponds to healthy tissue. In conditions of in vitro cell culture, normoxia may refer to a condition with a concentration of O₂ ranging from about 10 to about 21%. In specific embodiments, the O₂ concentration of a normoxic condition is about 15%, 16%, 17%, 18%, 19%, 20%, or 21%. In even more specific embodiments, the O₂ concentration is about 20% to 21%.

The term “indicator” as used herein refers to a result or representation of a result, including any information, number, ratio, signal, sign, mark, or note by which a skilled artisan can estimate and/or determine a likelihood or risk of whether or not a subject is suffering from a given disease or condition. In the case of the present invention, the “indicator” may optionally be used together with other clinical characteristics, to arrive at a diagnosis (that is, the occurrence or nonoccurrence) of a hypoxic condition or a G9a-associated disease or condition in a subject. That such an indicator is “determined” is not meant to imply that the indicator is 100% accurate. The skilled clinician may use the indicator together with other clinical indicia to arrive at a diagnosis.

The term “immobilized” means that a molecular species of interest is fixed to a solid support, suitably by covalent linkage. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physicochemical interactions typically occur in interactions between molecules. In particular embodiments, all that is required is that the molecules (e.g., nucleic acids or polypeptides) remain immobilized or attached to a support under conditions in which it is intended to use the support, for example in applications requiring nucleic acid amplification and/or sequencing or in antibody-binding assays. For example, oligonucleotides or primers are immobilized such that a 3′ end is available for enzymatic extension and/or at least a portion of the sequence is capable of hybridizing to a complementary sequence. In some embodiments, immobilization can occur via hybridization to a surface attached primer, in which case the immobilized primer or oligonucleotide may be in the 3′-5′ orientation. In other embodiments, immobilization can occur by means other than base-pairing hybridization, such as the covalent attachment.

As used herein, the term “label” and grammatical equivalents thereof, refer to any atom or molecule that can be used to provide a detectable and/or quantifiable signal. In particular, the label can be attached, directly or indirectly, to a nucleic acid or protein. Suitable labels that can be attached include, but are not limited to, radioisotopes, fluorophores, quenchers, chromophores, mass labels, electron dense particles, magnetic particles, spin labels, molecules that emit chemiluminescence, electrochemically active molecules, enzymes, cofactors, and enzyme substrates. A label can include an atom or molecule capable of producing a visually detectable signal when reacted with an enzyme. In some embodiments, the label is a “direct” label which is capable of spontaneously producing a detectible signal without the addition of ancillary reagents and is detected by visual means without the aid of instruments. For example, colloidal gold particles can be used as the label. Many labels are well known to those skilled in the art. In specific embodiments, the label is other than a naturally-occurring nucleoside. The term “label” also refers to an agent that has been artificially added, linked or attached via chemical manipulation to a molecule.

The “level” or “amount” of a biomarker is a detectable level or amount in a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment. In specific embodiments in which the level of a biomarker is “reduced” relative to a reference or control, the reduced level may refer to an overall reduction of any of at least about 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced level refers to a decrease in level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the level/amount of the respective biomarker in a reference sample, reference cell, reference tissue, control sample, control cell, or control tissue. In certain embodiments in which the level of a biomarker is “about the same” a reference or control, the level of biomarker varies by less than about 10%, 9%, 8%, 7%, 6%, 5%, 4%, 3%, 2%, 1%, 0.5%, 0.1%, or even less, as compared to the level of biomarker (e.g., protein or nucleic acid (e.g., gene or mRNA)), detected by standard art known methods such as those described herein, in a reference sample, reference cell, reference tissue, control sample, control cell, or control tissue.

The term “microarray” refers to an arrangement of hybridizable array elements, e.g., probes (including primers), ligands, biomarker nucleic acid sequence or protein sequences on a substrate.

The term “nucleic acid” or “polynucleotide” as used herein includes RNA, mRNA, miRNA, cRNA, cDNA mtDNA, or DNA. The term typically refers to a polymeric form of nucleotides of at least 10 bases in length, either ribonucleotides or deoxynucleotides or a modified form of either type of nucleotide. The term includes single and double stranded forms of DNA or RNA.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

As used herein, the term “positive response” means that the result of a treatment regimen includes some clinically significant benefit, such as the prevention, or reduction of severity, of symptoms, or a slowing of the progression of the condition. By contrast, the term “negative response” means that a treatment regimen provides no clinically significant benefit, such as the prevention, or reduction of severity, of symptoms, or increases the rate of progression of the condition.

“Protein”, “polypeptide” and “peptide” are used interchangeably herein to refer to a polymer of amino acid residues and to variants and synthetic analogues of the same.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Desirably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

As used herein, the term “probe” refers to a molecule that binds to a specific sequence or sub-sequence or other moiety of another molecule. Unless otherwise indicated, the term “probe” typically refers to a nucleic acid probe that binds to another nucleic acid, also referred to herein as a “target polynucleotide”, through complementary base pairing. Probes can bind target polynucleotides lacking complete sequence complementarity with the probe, depending on the stringency of the hybridization conditions. Probes can be labeled directly or indirectly and include primers within their scope.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The skilled artisan will understand that the term “prognosis” refers to an increased probability that a certain course or outcome will occur; that is, that a course or outcome is more likely to occur in a subject exhibiting a given condition, when compared to those individuals not exhibiting the condition.

As used herein, the term “quencher” includes any moiety that in close proximity to a donor fluorophore, takes up emission energy generated by the donor fluorophore and either dissipates the energy as heat or emits light of a longer wavelength than the emission wavelength of the donor fluorophore. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TAQMAN® probes while proximal quenching is used in molecular beacon and SCORPION® type probes. Suitable quenchers are selected based on the fluorescence spectrum of the particular fluorophore. Useful quenchers include, for example, the BLACK HOLE™ quenchers BHQ-1, BHQ-2, and BHQ-3 (Biosearch Technologies, Inc.), and the ATTO-series of quenchers (ATTO 540Q, ATTO 580Q, and ATTO 612Q; Atto-Tec GmbH).

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., faeces), tears, sweat, sebum, nipple aspirate, ductal lavage, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises cancer or tumor cells.

The term “solid support” as used herein refers to a solid inert surface or body to which a molecular species, such as a nucleic acid and polypeptides can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

The terms “subject”, “individual” and “patient” are used interchangeably herein to refer to an animal subject, particularly a vertebrate subject, and even more particularly a mammalian subject. Suitable vertebrate animals that fall within the scope of the invention include, but are not restricted to, any member of the phylum Chordata, subphylum vertebrata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards, etc.), and fish. A preferred subject is a primate (e.g., a human, ape, monkey, chimpanzee). The subject suitably has at least one (e.g., 1, 2, 3, 4, 5 or more) clinical sign of a hypoxic condition.

As used herein, the term “treatment regimen” refers to prophylactic and/or therapeutic (i.e., after onset of a specified condition) treatments, unless the context specifically indicates otherwise. The term “treatment regimen” encompasses natural substances and pharmaceutical agents (i.e., “drugs”) as well as any other treatment regimen including but not limited to dietary treatments, physical therapy or exercise regimens, surgical interventions, and combinations thereof.

It will be appreciated that the terms used herein and associated definitions are used for the purpose of explanation only and are not intended to be limiting.

### 2. Hypoxia Biomarkers

The present invention concerns methods, apparatus, compositions and kits for identifying the presence or absence of a hypoxic condition (e.g., a hypoxic cancer) in a subject, or for providing a prognosis for subjects with a disease or condition that is associated with a hypoxic condition. In particular, hypoxia biomarkers are disclosed for use in these modalities to assess the likelihood of the presence or absence of a hypoxic condition (e.g., a hypoxic cancer) in a subject, or for providing a prognosis for subjects with a disease or condition that is associated with a hypoxic condition. The methods, apparatus, compositions and kits of the invention are useful for early detection of a hypoxic condition (e.g., a hypoxic cancer) in a subject, thus allowing better treatment interventions for subjects with a disease or condition that is associated with the hypoxic condition.

The present inventors have determined that certain expression products are commonly, specifically and differentially expressed in a hypoxic condition in a subject when compared with a normoxic condition. The results presented herein provide clear evidence that a unique biologically-relevant biomarker profile predicts hypoxia with a remarkable degree of accuracy. This hypoxia biomarker profile was validated in an in vivo model (see, Example 5 for details). Overall, these findings provide compelling evidence that the expression products disclosed herein can function as biomarkers for hypoxia and may potentially serve as a useful diagnostic for triaging treatment decisions for subjects with a hypoxic condition, or a disease or condition that is associated with hypoxia. In this regard, it is proposed that the methods, apparatus, compositions and kits disclosed herein that are based on these biomarkers may serve in the point-of-care diagnostics that allow for rapid and inexpensive screening for hypoxia, which may result in significant cost savings to the medical system as subjects can be exposed to appropriate therapeutic agents that are suitable for treating a disease or condition that is associated with a hypoxic condition (e.g., a G9a antagonist) as opposed to therapeutic agents for diseases or conditions that are not associated with a hypoxic condition.

Thus, specific expression products are disclosed herein as hypoxia biomarkers that provide a means for identifying the presence or absence of a hypoxic condition in a subject. Evaluation of these hypoxia biomarkers through analysis of their levels in a subject or in a sample taken from a subject provides a measured or derived biomarker value for determining an indicator that can be used for assessing the presence or absence of a hypoxic condition in a subject or for providing a prognosis for a disease or condition that is associated with hypoxia in a subject.

Accordingly, biomarker values can be measured derived biomarker values, which are values that have been derived from one or more measured biomarker values, for example by applying a function to the one or more measured biomarker values. As used herein, biomarkers to which a function has been applied are referred to as “derived markers”.

The biomarker values may be determined in any one of a number of ways. An exemplary method of determining biomarker values is described by the present inventors in WO 2015/117204, which is incorporated herein by reference in its entirety. In one example, the process of determining biomarker values can include measuring the biomarker values, for example by performing tests on the subject or on sample(s) taken from the subject. More typically however, the step of determining the biomarker values includes having an electronic processing device receive or otherwise obtain biomarker values that have been previously measured or derived. This could include for example, retrieving the biomarker values from a data store such as a remote database, obtaining biomarker values that have been manually input, using an input device, or the like. The indicator is determined using a combination of the plurality of biomarker values, the indicator being at least partially indicative of the presence, or absence of a hypoxic condition. Assuming the method is performed using an electronic processing device, an indication of the indicator is optionally displayed or otherwise provided to the user. In this regard, the indication could be a graphical or alphanumeric representation of an indicator value. Alternatively, however, the indication could be the result of a comparison of the indicator value to predefined thresholds or ranges, or alternatively could be an indication of the presence or absence of a hypoxic condition, or prognosis for a disease or condition that is associated with hypoxia, derived using the indicator.

In some embodiments, biomarker values are combined, for example by adding, multiplying, subtracting, or dividing biomarker values to determine an indicator value. This step is performed so that multiple biomarker values can be combined into a single indicator value, providing a more useful and straightforward mechanism for allowing the indicator to be interpreted and hence used in diagnosing the presence or absence of a hypoxic condition in the subject, or providing a prognosis for a disease or condition that is associated with hypoxia in the subject.

In some embodiments in which a plurality of biomarkers and biomarker values are used, in order to ensure that an effective diagnosis or prognosis can be determined, at least two of the biomarkers have a mutual correlation in respect of hypoxia that lies within a mutual correlation range, the mutual correlation range being between ±0.9. This requirement means that the two biomarkers are not entirely correlated in respect of each other when considered in the context of the hypoxic condition (e.g., hypoxic cancer) being diagnosed. In other words, at least two of the biomarkers in the combination respond differently as the condition changes, which adds significantly to their ability when combined to discriminate between at least two conditions, to diagnose the presence or absence of a hypoxic condition, and/or to provide a prognosis for the disease or condition that is associated with hypoxia in the subject.

Typically, the requirement that biomarkers have a low mutual correlation means that the biomarkers may relate to different biological attributes or domains such as, but not limited, to different molecular functions, different biological processes and different cellular components. Illustrative examples of molecular function include addition of, or removal of, one of more of the following moieties to, or from, a protein, polypeptide, peptide, nucleic acid (e.g., DNA, RNA): linear, branched, saturated or unsaturated alkyl (e.g., C1-C24 alkyl); phosphate; ubiquitin; acyl; fatty acid, lipid, phospholipid; nucleotide base; hydroxyl and the like. Molecular functions also include signaling pathways, including without limitation, receptor signaling pathways and nuclear signaling pathways. Non-limiting examples of molecular functions also include cleavage of a nucleic acid, peptide, polypeptide or protein at one or more sites; polymerization of a nucleic acid, peptide, polypeptide or protein; translocation through a cell membrane (e.g., outer cell membrane; nuclear membrane); translocation into or out of a cell organelle (e.g., Golgi apparatus, lysosome, endoplasmic reticulum, nucleus, mitochondria); receptor binding, receptor signaling, membrane channel binding, membrane channel influx or efflux; and the like.

Illustrative examples of biological processes include: stages of the cell cycle such as meiosis, mitosis, cell division, prophase, metaphase, anaphase, telophase and interphase, stages of cell differentiation; apoptosis; necrosis; chemotaxis; immune responses including adaptive and innate immune responses, pro-inflammatory immune responses, autoimmune responses, tolerogenic responses and the like. Other illustrative examples of biological processes include generating or breaking down adenosine triphosphate (ATP), saccharides, polysaccharides, fatty acids, lipids, phospholipids, sphingolipids, glycolipids, cholesterol, nucleotides, nucleic acids, membranes (e.g., cell plasma membrane, nuclear membrane), amino acids, peptides, polypeptides, proteins and the like. Representative examples of cellular components include organelles, membranes, as for example noted above, and others.

It will be understood that the use of biomarkers that have different biological attributes or domains provides further information than if the biomarkers were related to the same or common biological attributes or domains. In this regard, it will be appreciated if the at least two biomarkers are highly correlated to each other, the use of both biomarkers would add little diagnostic/prognostic improvement compared to the use of a single one of the biomarkers. Accordingly, an indicator-determining method of the present invention in which a plurality of biomarkers and biomarker values are used preferably employ biomarkers that are not well correlated with each other, thereby ensuring that the inclusion of each biomarker in the method adds significantly to the discriminative ability of the indicator.

Despite this, in order to ensure that the indicator can accurately be used in performing the discrimination between the presence or absence of a hypoxic condition (e.g., a hypoxic cancer) or the provision of a prognosis for a disease or condition that is associate with hypoxia, the indicator has a performance value that is greater than or equal to a performance threshold. The performance threshold may be of any suitable form but is to be typically indicative of an explained variance of at least 0.3, or an equivalent value of another performance measure.

Suitably, a combination of biomarkers is employed, which biomarkers have a mutual correlation between ±0.9 and which combination provides an explained variance of at least 0.3. This typically allows an indicator to be defined that is suitable for ensuring that an accurate diagnosis or prognosis can be obtained whilst minimizing the number of biomarkers that are required. Typically, the mutual correlation range is one of ±0.8; ±0.7; ±0.6; ±0.5; ±0.4; ±0.3; ±0.2; and, ±0.1. Typically, each hypoxia biomarker has a condition correlation with the presence or absence of a hypoxic condition (e.g., hypoxic cancer) or with a prognosis for a disease or condition that is associated with hypoxia, that lies outside a condition correlation range, the condition correlation range being between ±0.3 and more typically ±0.9; ±0.8; ±0.7; ±0.6; ±0.5; and, ±0.4. Typically, the performance threshold is indicative of an explained variance of at least one of 0.4; 0.5; 0.6; 0.7; 0.8; and 0.9.

It will be understood that in this context, the biomarkers used within the above-described method can define a biomarker profile for a hypoxic condition which includes a minimal number of biomarkers, whilst maintaining sufficient performance to allow the biomarker profile to be used in making a clinically relevant diagnosis, prognosis, or differentiation. Minimizing the number of biomarkers used minimizes the costs associated with performing diagnostic or prognostic tests and in the case of nucleic acid expression products, allows the test to be performed utilizing relatively straightforward techniques such as nucleic acid array, and polymerase chain reaction (PCR) processes, or the like, allowing the test to be performed rapidly in a clinical environment.

Furthermore, producing a single indicator value allows the results of the test to be easily interpreted by a clinician or other medical practitioner, so that test can be used for reliable diagnosis in a clinical environment.

Processes for generating suitable biomarker profiles are described for example in WO 2015/117204, which uses the term “biomarker signature” in place of “biomarker profile” as defined herein. It will be understood, therefore, that terms “biomarker profile” and “biomarker signature” are equivalent in scope. The biomarker profile-generating processes disclosed in WO 2015/117204 provide mechanisms for selecting a combination of biomarkers, and more typically derived biomarkers, that can be used to form a biomarker profile, which in turn can be used in diagnosing the presence or absence of a hypoxic condition (e.g., a hypoxic cancer) or in providing a prognosis for a disease or condition that is associated with hypoxia. In this regard, the biomarker profile defines the biomarkers that should be measured (i.e., the profile biomarkers), how derived biomarker values should be determined for measured biomarker values, and then how biomarker values should be subsequently combined to generate an indicator value. The biomarker profile can also specify defined indicator value ranges that indicate a particular presence or absence of a hypoxic condition (e.g., hypoxic cancer) or that provide a prognosis for a disease or condition that is associated with hypoxia.

Using the above-described methods a number of biomarkers have been identified that are particularly useful for assessing a likelihood of the presence or absence of a hypoxic condition in a subject or for providing a prognosis for a disease or condition that is associated with hypoxia. These biomarkers are referred to herein as “hypoxia biomarkers”. As used herein, the term “hypoxia biomarker” refers to a biomarker of the host which is altered, or whose level of expression is altered, as part of a response to damage or insult resulting from a decreased concentration of oxygen (02), relative to normal tissue. The hypoxia biomarkers are suitably expression products of genes (also referred to interchangeably herein as “hypoxia biomarker genes”), including polynucleotide and polypeptide expression products. As used herein, polynucleotide expression products of hypoxia biomarker genes are referred to herein as “hypoxia biomarker polynucleotides”. Polypeptide expression products of the hypoxia biomarker genes are referred to herein as “hypoxia biomarker polypeptides.”

Hypoxia biomarkers are suitably selected from expression products of any one or more of the following hypoxia genes: AGBL3, AGMO, AGTR1, ALS2CR12, ALX1, ANAPC5, ANKRD20A1, ANXA13, AP1AR, ARHGAP10, ARMCX1, ARNTL, C10orf25, C14orf169, C18orf34, C18orf62, C1QTNF9, C2orf76, C6orf 165, C7orf45, C7orf62, C9orf131, C9orf85, CA10, CAPNS2, CCDC121, CCDC141, CD1C, CDH11, CEACAM7, CEP170P1, CYTIP, DBF4, DDX56, DPPA2, DPPA3P2, DTX2, EIF4G2, ELMO1, ELOVL3, ENAM, FAM172BP, FCGR1A, FFAR2, FGF12, FGFR2, FLJ42102, FLJ44838, FLJ45721, FN1, FOXP2, GATA2, GDI2, GGTLC2, GK2, GPR78, HARBI1, HEMGN, HHEX, HIST1H2AA, HMGN2, HMGN2P28, HMGN2P46, HOXC13, HPDL, HRCT1, HRH1, HS3ST3B1, HSD3B2, ICA1L, IDO2, IL10, IL17A, IL18, IL1B, KARS, KBTBD6, KCNA2, KLB, KLRG1, KMO, KRT10, KRT222, LCN1, LIG4, LINC00312, LINC00328, LINC00596, LLPH, LOC100049716, LOC100287879, LOC221442, LOC642947, LOC644714, LRRC37A5P, LRRC53, LTA, MEIG1, METTL2A, MGAT2, MGC23270, MILR1, MIR148A, MIR218-1, MIR34B, MIR494, MIR516B2, MMP16, MMP27, MPP1, MRPL30, MUTYH, MYH8, NBLA00301, NCRNA00185, ND6, NEFM, NEUROD1, NONO, NPCDR1, NPY5R, OCM, OCM2, OGN, OR10A2, OR2A12, OR2D3, OR2M3, OR2T2, OR2T35, OR4D10, OR4D2, OR5J2, OR5M10, OR8H3, OR9G1, ORM2, OTOG, PAGE2B, PLAG1, PRL, PRPF8, PSCA, PTPN20A, RBM44, RBMY1C, RBMY1F, RFK, RIMBP3, RN18S1, RN5S1, RN7SK, RN7SL1, RNF152, RNF185, RNPS1, RNU11, RNU1-4, RNU2-1, RNU4-2, RNU5F-1, RNU6-2, RNY1, RP11-165H20.1, RPL21P68, RPS27AP17, S1PR3, SART1, SDAD1, SEPP1, SETD9, SIGLEC14, SKAP1, SLC36A3, SLC8A1, SLC9B1P2, SLCO4C1, SMNDC1, SNORA1, SNORA72, SNORA75, SNORD114-2, SNORD115-10, SNORD116-19, SNORD18A, SNORD3B-1, SPOCK3, ST6GAL1, STAMBPL1, TAF1D, TAS2R14, TBCCD1, TCEB3B, TMEM100, TMIGD1, TOB2, TUBB8, UBD, UGT2B17, UQCRFS1, VNN1, ZC3HC1, ZCCHC12, ZNF222, ZNF259, ZNF439, ZNF487P, ZNF569, ZNF678, ZNF684, ZNF716, ZNF718, ZNRF3, ZSCAN29, and CDH10. Non-limiting examples of nucleotide sequences for these hypoxia biomarkers are listed in SEQ ID NOs: 1-201. Non-limiting examples of corresponding amino acid sequences for these hypoxia biomarkers are listed in SEQ ID NOs: 202-367.

In more specific embodiments, the one or more hypoxia genes are selected from ARNTL, CD1C, HHEX, KLRG1, MMP16, FGFR2, GATA2, CEACAM7, OGN, and AGTR1. More specifically, 1, 2, 3, 4, or 5 hypoxia biomarkers may be selected from ARNTL, CEACAM7, GATA2, HHEX, KLRG1, and OGN.

In some of the same embodiments and other embodiments, two or more hypoxia biomarkers may be selected from Table 1.

TABLE 1

| Biomarker 1 | Biomarker 2 |
|---|---|
| ARNTL | CD1C, HHEX, KLRG1, MMP16, |
| | FGFR2, GATA2, CEACAM7, OGN, |
| | AGTR1 |
| CD1C | HHEX, KLRG1, MMP16, FGFR2, |
| | GATA2, CEACAM7, OGN, AGTR1 |
| HHEX | KLRG1, MMP16, FGFR2, GATA2, |
| | CEACAM7, OGN, AGTR1 |
| KLRG1 | MMP16, FGFR2, GATA2, CEACAM7, |
| | OGN, AGTR1 |
| MMP16 | FGFR2, GATA2, CEACAM7, OGN, |
| | AGTR1 |
| FGFR2 | GATA2, CEACAM7, OGN, AGTR1 |
| GATA2 | CEACAM7, OGN, AGTR1 |
| KLRG1 | OGN, AGTR1 |
| OGN | AGTR1 |

In some of the same embodiments and other embodiments, two or more hypoxia biomarkers may be selected from Table 2.

TABLE 2

| Biomarker 1 | Biomarker 2 | Biomarker 3 |
|---|---|---|
| ARNTL | CD1C | HHEX, KLRG1, MMP16, FGFR2, |
| | | GATA2, CEACAM7, OGN, AGTR1 |
| | HHEX | KLRG1, MMP16, FGFR2, GATA2, |
| | | CEACAM7, OGN, AGTR1 |
| | KLRG1 | MMP16, FGFR2, GATA2, |
| | | CEACAM7, OGN, AGTR1 |
| | MMP16 | FGFR2, GATA2, CEACAM7, OGN, |
| | | AGTR1 |
| | FGFR2 | GATA2, CEACAM7, OGN, AGTR1 |
| | GATA2 | CEACAM7, OGN, AGTR1 |
| | CEACAM7 | OGN, AGTR1 |
| | OGN | AGTR1 |
| CD1C | HHEX | KLRG1, MMP16, FGFR2, GATA2, |
| | | CEACAM7, OGN, AGTR1 |
| | KLRG1 | MMP16, FGFR2, GATA2, |
| | | CEACAM7, OGN, AGTR1 |
| | MMP16 | FGFR2, GATA2, CEACAM7, OGN, |
| | | AGTR1 |
| | FGFR2 | GATA2, CEACAM7, OGN, AGTR1 |
| | GATA2 | CEACAM7, OGN, AGTR1 |
| | CEACAM7 | OGN, AGTR1 |
| | OGN | AGTR1 |
| HHEX | KLRG1 | MMP16, FGFR2, GATA2, |
| | | CEACAM7, OGN, AGTR1 |
| | MMP16 | FGFR2, GATA2, CEACAM7, OGN, |
| | | AGTR1 |
| | FGFR2 | GATA2, CEACAM7, OGN, AGTR1 |
| | GATA2 | CEACAM7, OGN, AGTR1 |
| | CEACAM7 | OGN, AGTR1 |
| | OGN | AGTR1 |
| KLRG1 | MMP16 | FGFR2, GATA2, CEACAM7, OGN, |
| | | AGTR1 |
| | FGFR2 | GATA2, CEACAM7, OGN, AGTR1 |
| | GATA2 | CEACAM7, OGN, AGTR1 |
| | CEACAM7 | OGN, AGTR1 |
| | OGN | AGTR1 |
| MMP16 | FGFR2 | GATA2, CEACAM7, OGN, AGTR1 |
| | GATA2 | CEACAM7, OGN, AGTR1 |
| | CEACAM7 | OGN, AGTR1 |
| | OGN | AGTR1 |
| FGFR2 | GATA2 | CEACAM7, OGN, AGTR1 |
| | CEACAM7 | OGN, AGTR1 |
| | OGN | AGTR1 |
| GATA2 | CEACAM7 | OGN, AGTR1 |
| | OGN | AGTR1 |
| KLRG1 | OGN | AGTR1 |

In some of the same embodiments and other embodiments, four or more hypoxia biomarkers may be selected from Table 3.

TABLE 3

| Biomarker 1 | Biomarker 2 | Biomarker 3 | Biomarker 4 |
|---|---|---|---|
| ARNTL | CD1C | HHEX | KLRG1, MMP16, FGFR2, |
| | | | GATA2, CEACAM7, OGN, |
| | | | AGTR1 |
| | | KLRG1 | MMP16, FGFR2, GATA2, |
| | | | CEACAM7, OGN, AGTR1 |
| | | MMP16 | FGFR2, GATA2, |
| | | | CEACAM7, OGN, AGTR1 |
| | | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | HHEX | KLRG1 | MMP16, FGFR2, GATA2, |
| | | | CEACAM7, OGN, AGTR1 |
| | | MMP16 | FGFR2, GATA2, |
| | | | CEACAM7, OGN, AGTR1 |
| | | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | KLRG1 | MMP16 | FGFR2, GATA2, |
| | | | CEACAM7, OGN, AGTR1 |
| | | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | MMP16 | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | FGFR2 | GATA2 | CEACAM7, OGN, AGTR1 |
| | | CEACAM7 | OGN, AGTR1 |
| | | OGN | AGTR1 |
| | GATA2 | CEACAM7 | OGN, AGTR1 |
| | | OGN | AGTR1 |
| | CEACAM7 | OGN | AGTR1 |
| CD1C | HHEX | KLRG1 | MMP16, FGFR2, GATA2, |
| | | | CEACAM7, OGN, AGTR1 |
| | | MMP16 | FGFR2, GATA2, |
| | | | CEACAM7, OGN, AGTR1 |
| | | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | KLRG1 | MMP16 | FGFR2, GATA2, |
| | | | CEACAM7, OGN, AGTR1 |
| | | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | MMP16 | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | FGFR2 | GATA2 | CEACAM7, OGN, AGTR1 |
| | | CEACAM7 | OGN, AGTR1 |
| | | OGN | AGTR1 |
| | GATA2 | CEACAM7 | OGN, AGTR1 |
| | | OGN | AGTR1 |
| | CEACAM7 | OGN | AGTR1 |
| HHEX | KLRG1 | MMP16 | FGFR2, GATA2, |
| | | | CEACAM7, OGN, AGTR1 |
| | | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | MMP16 | FGFR2 | GATA2, CEACAM7, OGN, |
| | | | AGTR1 |
| | | GATA2 | CEACAM7, OGN, AGTR1 |
| | | CEACAM7 | OGN, AGTR1 |
| | | OGN | AGTR1 |
| | FGFR2 | GATA2 | CEACAM7, OGN, AGTR1 |
| | | CEACAM7 | OGN, AGTR1 |
| | | OGN | AGTR1 |
| | GATA2 | CEACAM7 | OGN, AGTR1 |
| | | OGN | AGTR1 |
| | CEACAM7 | OGN | AGTR1 |
| KLRG1 | MMP16 | FGFR2 | GATA2, CEACAM7, |
| | | | OGN, AGTR1 |
| | | GATA2 | CEACAM7, OGN, AGTR1 |
| | | CEACAM7 | OGN, AGTR1 |
| | | OGN | AGTR1 |
| | FGFR2 | GATA2 | CEACAM7, OGN, AGTR1 |
| | | CEACAM7 | OGN, AGTR1 |
| | | OGN | AGTR1 |
| | GATA2 | CEACAM7 | OGN, AGTR1 |
| | | OGN | AGTR1 |
| | CEACAM7 | OGN | AGTR1 |
| MMP16 | FGFR2 | GATA2 | CEACAM7, OGN, AGTR1 |
| | | CEACAM7 | OGN, AGTR1 |
| | | OGN | AGTR1 |
| | GATA2 | CEACAM7 | OGN, AGTR1 |
| | | OGN | AGTR1 |
| | CEACAM7 | OGN | AGTR1 |
| FGFR2 | GATA2 | CEACAM7 | OGN, AGTR1 |
| | | OGN | AGTR1 |
| | CEACAM7 | OGN | AGTR1 |
| GATA2 | CEACAM7 | OGN | AGTR1 |

In some of the same embodiments and other embodiments, five or more hypoxia biomarkers may be selected from Table 4.

TABLE 4

| Bio- | Bio- | Bio- | Bio- | Bio- |
|---|---|---|---|---|
| marker 1 | marker 2 | marker 3 | marker 4 | marker 5 |
| ARNTL | CD1C | HHEX | KLRG1 | MMP16, FGFR2, |
| | | | | GATA2, |
| | | | | CEACAM7, |
| | | | | OGN, AGTR1 |
| | | | MMP16 | FGFR2, GATA2, |
| | | | | CEACAM7, OGN, |
| | | | | AGTR1 |
| | | | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | KLRG1 | MMP16 | FGFR2, GATA2, |
| | | | | CEACAM7, OGN, |
| | | | | AGTR1 |
| | | | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | MMP16 | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | FGFR2 | GATA2, | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | HHEX | KLRG1 | MMP16 | FGFR2, GATA2, |
| | | | | CEACAM7, OGN, |
| | | | | AGTR1 |
| | | | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | MMP16 | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | KLRG1 | MMP16 | FGFR | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | MMP16 | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | FGFR2 | GATA2 | CEACAM7 | OGN, AGTR1 |
| | | | OGN | AGTR1 |
| | | CEACAM7 | OGN | AGTR1 |
| | GATA2 | CEACAM7 | OGN | AGTR1 |
| CD1C | HHEX | KLRG1 | MMP16 | FGFR2, GATA2, |
| | | | | CEACAM7, OGN, |
| | | | | AGTR1 |
| | | | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | MMP16 | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | KLRG1 | MMP16 | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | MMP16 | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | FGFR2 | GATA2 | CEACAM7 | OGN, AGTR1 |
| | | | OGN | AGTR1 |
| | | CEACAM7 | OGN | AGTR1 |
| | GATA2 | CEACAM7 | OGN | AGTR1 |
| HHEX | KLRG1 | MMP16 | FGFR2 | GATA2, |
| | | | | CEACAM7, |
| | | | | OGN, AGTR1 |
| | | | GATA2 | CEACAM7, OGN, |
| | | | | AGTR1 |
| | | | CEACAM7 | OGN, AGTR1 |
| | | | OGN | AGTR1 |
| | | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | MMP16 | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | FGFR2 | GATA2 | CEACAM7 | OGN, AGTR1 |
| | | | OGN | AGTR1 |
| | | CEACAM7 | OGN | AGTR1 |
| | GATA2 | CEACAM7 | OGN | AGTR1 |
| KLRG1 | MMP16 | FGFR2 | GATA2 | CEACAM7, OGN, |
| | | | | AGTR1 |
| | | | CEACAM7 | OGN, AGTR1 |
| | | | OGN | AGTR1 |
| | | GATA2 | CEACAM7 | OGN, AGTR1 |
| | | | OGN | AGTR1 |
| | | CEACAM7 | OGN | AGTR1 |
| | FGFR2 | GATA2 | CEACAM7 | OGN, AGTR1 |
| | | | OGN | AGTR1 |
| | | CEACAM7 | OGN | AGTR1 |
| | GATA2 | CEACAM7 | OGN | AGTR1 |
| MMP16 | FGFR2 | GATA2 | CEACAM7 | OGN, AGTR1 |
| | | | OGN | AGTR1 |
| | | CEACAM7 | OGN | AGTR1 |
| | GATA2 | CEACAM7 | OGN | AGTR1 |
| FGFR2 | GATA2 | CEACAM7 | OGN | AGTR1 |

By way of an illustrative example, in some instances of the present invention at least 6 hypoxia biomarkers may be measured, wherein the at least 6 hypoxia biomarkers comprise, consist or consist essentially of ARNTL, CEACAM7, GATA2, HHEX, KLRG1, and OGN.

In these examples, the indicator-determining methods suitably include determining a pair of biomarker values, wherein each biomarker value is a value measured or derived for at least one corresponding hypoxic biomarker of the subject and is at least partially indicative of a concentration of the hypoxic biomarker in a sample taken from the subject. The biomarker values are typically used to determine a derived biomarker value using the pair of biomarker values, wherein the derived biomarker value is indicative of a ratio of concentrations of the pair of hypoxic biomarkers. Thus, if the biomarker values denote the concentrations of the hypoxic biomarkers, then the derived biomarker value will be based on a ratio of the biomarker values. However, if the biomarker values are related to the concentrations of the biomarkers, for example if they are logarithmically related by virtue of the biomarker values being based on PCR cycle times, or the like, then the biomarker values may be combined in some other manner, such as by subtracting the cycle times to determine a derived biomarker value indicative of a ratio of the concentrations of the hypoxic biomarkers.

The derived biomarker value is then used to determine the indicator, either by using the derived biomarker value as an indicator value, or by performing additional processing, such as comparing the derived biomarker value to a reference or the like, as will be described in more detail below.

In some embodiments in which pairs of hypoxia biomarkers are used to determine a derived biomarker value, one biomarker of a biomarker pair is selected from Group 1 hypoxia biomarkers and the other is selected from Group 2 hypoxia biomarkers, wherein an individual Group 1 hypoxia biomarker is an expression product of a gene selected from the group consisting of: ARNTL, CD1C, HHEX, KLRG1, MMP16, FGFR2, GATA2, CEACAM7, OGN, and AGTR1; and wherein an individual Group 2 hypoxia biomarker is an expression product of G9a.

The derived biomarker values could be combined using a combining function such as an additive model; a linear model; a support vector machine; a neural network model; a tree-learning method (e.g., random forest model); a regression model; a genetic algorithm; an annealing algorithm; a weighted sum; a nearest neighbor model; an ensemble method (e.g., bagging, boosting weighted averaging); and a probabilistic model. In some embodiments, biomarker values are measured or derived for a Group 1 hypoxia biomarker and for a Group 2 hypoxia biomarker, and the indicator is determined by combining the biomarker values.

In some embodiments, the indicator is compared to an indicator reference, with a likelihood being determined in accordance with results of the comparison. The indicator reference may be derived from indicators determined for a number of individuals in a reference population. The reference population typically includes individuals having different characteristics, such as a plurality of individuals of different sexes; and/or ethnicities, with different groups being defined based on different characteristics, with the subject's indicator being compared to indicator references derived from individuals with similar characteristics. The reference population can also include a plurality of healthy individuals, a plurality of individuals with hypoxic condition (e.g., hypoxic cancer), a plurality of individuals with a non-hypoxic G9a-associated disease or condition, a plurality of individuals showing clinical signs of a hypoxic condition, a plurality of individuals showing clinical signs of a non-hypoxic G9a associated disease or condition.

The indicator can also be used for determining a likelihood of the subject having a first or second condition, wherein the first condition is a hypoxic condition (e.g., a hypoxic cancer) and the second condition is a normoxic condition; in other words, to distinguish between these conditions. In this case, this would typically be achieved by comparing the indicator to first and second indicator references, the first and second indicator references being indicative of first and second conditions and determining the likelihood in accordance with the results of the comparison. In particular, this can include determining first and second indicator probabilities using the results of the comparisons and combining the first and second indicator probabilities, for example using a Bayes method, to determine a condition probability corresponding to the likelihood of the subject having one of the conditions. In this situation the first and second conditions could include a hypoxic cancer and another non-hypoxic cancer, or hypoxia and normoxia. In this case, the first and second indicator references are distributions of indicators determined for first and second groups of a reference population, the first and second group consisting of individuals diagnosed with the first or second condition respectively.

In specific embodiments, the indicator-determining methods of the present invention are performed using at least one electronic processing device, such as a suitably programmed computer system or the like. In this case, the electronic processing device typically obtains at least one measured biomarker values, either by receiving these from a measuring or other quantifying device, or by retrieving these from a database or the like. The processing device then determines a first derived biomarker value indicative of a first hypoxia biomarker. and optionally, a second derived biomarker value indicative of a second hypoxia biomarker. In some of the same embodiments and other embodiments, the first derived biomarker value is value indicative of a ratio of first and second hypoxia biomarkers. In instances where more than one biomarker value is derived, the processing device may then determine the indicator by combining the first and second (and optionally third, fourth, fifth, etc.) derived biomarker values, as appropriate.

The processing device can then generate a representation of the indicator, for example by generating an alphanumeric indication of the indicator, a graphical indication of a comparison of the indicator to one or more indicator references or an alphanumeric indication of a likelihood of the subject having at least one medical condition.

The indicator-determining methods of the present invention typically include obtaining a sample from a subject, who typically has at least one clinical sign of a hypoxic condition (e.g., a hypoxic cancer), wherein the sample includes one or more hypoxia biomarkers (e.g., polynucleotide or polypeptide expression products of hypoxia genes) and quantifying at least two (e.g., 2, 3, 4, 5, 6, 7, 8, 9, 10 or more) of the hypoxia biomarkers within the sample to determine biomarker values. This can be achieved using any suitable technique, and will depend on the nature of the hypoxia biomarkers. Suitably, an individual measured or derived hypoxia biomarker value corresponds to the level, abundance or amount of a respective hypoxia biomarker or to a function that is applied to that level or amount. As used herein the terms “level”, “abundance” and “amount” are used interchangeably herein to refer to a quantitative amount (e.g., weight or moles), a semi-quantitative amount, a relative amount (e.g., weight % or mole % within class), a concentration, and the like. Thus, these terms encompass absolute or relative amounts or concentrations of hypoxia biomarkers in a sample. For example, if the indicator in some embodiments of the indicator-determining method of the present invention, which uses a plurality of hypoxia biomarkers, is based on a ratio of concentrations of the polynucleotide expression products, this process would typically include quantifying polynucleotide expression products by amplifying at least some polynucleotide expression products in the sample, determining an amplification amount representing a degree of amplification required to obtain a defined level of each of a pair of polynucleotide expression products and determining the indicator by determining a difference between the amplification amounts. In this regard, the amplification amount is generally a cycle time, a number of cycles, a cycle threshold and an amplification time. For example, in some embodiments the method includes determining a first derived biomarker value by determining the amplification amount of a first polynucleotide expression product, determining a second derived biomarker value by determining the amplification amounts of a second polynucleotide expression product, and determining the indicator by combining the first and second derived biomarker values.

In some embodiments, the presence or absence of a hypoxic condition (e.g., a hypoxic cancer) or prognosis for a disease or condition that is associated with hypoxia in a subject is established by determining two or more hypoxic biomarker values, wherein a hypoxic biomarker value is indicative of a value derived for hypoxia biomarkers in a subject or in a sample taken from the subject. These biomarkers are referred to herein as “sample hypoxia biomarkers”. In accordance with the present invention, a sample hypoxia biomarker corresponds to a reference hypoxia biomarker (also referred to herein as a “corresponding hypoxia biomarker”). By “corresponding hypoxia biomarker” is meant a hypoxia biomarker that is structurally and/or functionally similar to a reference hypoxia biomarker as set forth for example in Table 5. Representative corresponding hypoxia biomarkers include expression products of allelic variants (same locus), homologues (different locus), and orthologues (different organism) of reference hypoxia biomarker genes. Nucleic acid variants of reference hypoxia biomarker genes and encoded hypoxia biomarker polynucleotide expression products can contain nucleotide substitutions, deletions, inversions and/or insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of a reference hypoxia polypeptide.

Generally, variants of a particular hypoxia biomarker gene or polynucleotide will have at least about 40%, 45%, 50%, 51%, 52%, 53%, 54%, 55%, 56%, 57%, 58%, 59% 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to that particular nucleotide sequence as determined by sequence alignment programs known in the art using default parameters. In some embodiments, the hypoxia biomarker gene or polynucleotide displays at least about 40%, 45%, 50%, 51%, 52%, 53%, 54%, 55%, 56%, 57%, 58%, 59% 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any one of the hypoxia biomarkers listed in Table 5.

TABLE 5

| Gene biomarker/GenBank Acc. No | SEQ ID NO: |
|---|---|
| ARNTL/NM_001178 | 1 |
| CD1C/NM_001178 | 2 |
| HHEX/NM_002729 | 3 |
| KLRG1/NM_001329099 | 4 |
| MMP16/NM_005941 | 5 |
| FGFR2/NM_000141 | 6 |
| GATA2/NM_001145661 | 7 |
| CEACAM7/NM_001291485 | 8 |
| OGN/NM_024416 | 9 |
| AGTR1/NM_000685 | 10 |
| ARMCX1/NM_016608 | 11 |
| C10orf25/XM_498436 | 12 |
| C14orf169/XM_012963799 | 13 |
| C18orf34/NM_001105528 | 14 |
| C18orf62/NM_001037331 | 15 |
| C1QTNF9/NM_001303137 | 16 |
| C2orf76/NM_001017927 | 17 |
| C6orf165/NM_001031743 | 18 |
| C7orf45/NM_145268 | 19 |
| C7orf62/NM_152706 | 20 |
| C9orf131/NM_001040412 | 21 |
| C9orf85/NM_182505 | 22 |
| CA10/NM_001082533 | 23 |
| CAPNS2/NM_032330 | 24 |
| CCDC121/NM_001142683 | 25 |
| CCDC141/NM_001316745 | 26 |
| CDH10/NM_006727 | 27 |
| CDH11/NM_001308392 | 28 |
| CEP170P1/NR_003135 | 29 |
| CYTIP/NM_004288 | 30 |
| DBF4/NM_001180360 | 31 |
| DDX56/NM_019082 | 32 |
| DPPA2/NM_138815 | 33 |
| DPPA3P2/NC_000014 | 34 |
| DTX2/NM_001102594 | 35 |
| EIF4G2/NM_001172705 | 36 |
| ELMO1/NM_001206480 | 37 |
| ELOVL3/NM_152310 | 38 |
| ENAM/NM_031889 | 39 |
| FAM172BP/NR_036433 | 40 |
| FCGR1A/NM_000566 | 41 |
| FFAR2 | 42 |
| FGF12/NM_005306 | 43 |
| FLJ42102/NR_038862 | 44 |
| FLJ44838/NM_001082575 | 45 |
| FLJ45721/NM_207490 | 46 |
| FN1/NM_001306129 | 47 |
| FOXP2/NM_014491 | 48 |
| GDI2/NM_001494 | 49 |
| GGTLC2/NM_199127 | 50 |
| GK2/NM_033214 | 51 |
| GPR78/NM_080819 | 52 |
| HARBI1/NM_173811 | 53 |
| HEMGN/NM_197978 | 54 |
| HIST1H2AA/NM_170745 | 55 |
| HMGN2/NM_005517 | 56 |
| HMGN2P28/NC_000006 | 57 |
| HMGN2P46/NR_022014 | 58 |
| HOXC13/NM_017410 | 59 |
| HPDL/NM_032756 | 60 |
| HRCT1/NM_001039792 | 61 |
| HRH1/NM_000861 | 62 |
| HS3ST3B1/NM_006041 | 63 |
| HSD3B2/NM_000198 | 64 |
| ICA1L/NM_001288622 | 65 |
| IDO2/NM_194294 | 66 |
| IL10/NM_000572 | 67 |
| IL17A/NM_002190 | 68 |
| IL18/NM_001243211 | 69 |
| IL1B/NM_000576 | 70 |
| KARS/NM_001130089 | 71 |
| KBTBD6/NM_152903 | 72 |
| KCNA2/NM_001204269 | 73 |
| KLB/NM_175737 | 74 |
| KMO/NM_003679 | 75 |
| KRT10/NM_000421 | 76 |
| KRT222/NM_152349 | 77 |
| LCN1/NM_002297 | 78 |
| LIG4/NM_001098268 | 79 |
| LINC00312/NR_024065 | 80 |
| LINC00328/AL391382 | 81 |
| LINC00596/NR_024081 | 82 |
| LLPH/NM_032338 | 83 |
| LOC100049716/NR_122124 | 84 |
| LOC100287879/NR_033978 | 85 |
| LOC642947/NM_001039895 | 86 |
| LRRC37A5P/NR_034087 | 87 |
| LRRC53/XM_017003080 | 88 |
| LTA/NM_000595 | 89 |
| MEIG1/NM_001080836 | 90 |
| METTL2A/NM_181725 | 91 |
| MGAT2/NM_181725 | 92 |
| MILR1/NM_001085423 | 93 |
| MIR148A/NR_029597 | 94 |
| MIR218-1/NR_029631 | 95 |
| MIR34B/NR_029839 | 96 |
| MIR494/NR_030174 | 97 |
| MIR516B2/NR_030174 | 98 |
| MMP27/NM_022122 | 99 |
| MPP1/NM_002436 | 100 |
| MRPL30/NM_145212 | 101 |
| MUTYH/NM_012222 | 102 |
| MYH8/NM_002472 | 103 |
| NBLA00301/NR_003679 | 104 |
| NCRNA00185/NR_125733 | 105 |
| ND6/J01415.2 | 106 |
| NEFM/NM_001105541 | 107 |
| NEUROD1/NM_002500 | 108 |
| NONO/NM_001145408 | 109 |
| NPY5R/NM_001317091 | 110 |
| OCM/NM_001097622 | 111 |
| OCM2/NM_006188 | 112 |
| OR10A2/NM_001004460 | 113 |
| OR2A12/NM_001004135 | 114 |
| OR2D3/NM_001004684 | 115 |
| OR2M3/NM_001004689 | 116 |
| OR2T2/NM_001004136 | 117 |
| OR2T35/NM_001001827 | 118 |
| OR4D10/NM_001004705 | 119 |
| OR4D2/NM_001004707 | 120 |
| OR5J2/NM_001005492 | 121 |
| OR5M10/NM_001004741 | 122 |
| OR8H3/NM_001005201 | 123 |
| OR9G1/NM_001005213 | 124 |
| ORM2/NM_000608 | 125 |
| OTOG/NM_001277269 | 126 |
| PAGE2B/NM_001015038 | 127 |
| PLAG1/NM_001114634 | 128 |
| PRL/NM_000948 | 129 |
| PRPF8/NM_006445 | 130 |
| PSCA/NM_005672 | 131 |
| PTPN20A/NM_001042357 | 132 |
| RBM44/NM_001080504 | 133 |
| RBMY1F/NM_152585 | 134 |
| RFK/NM_018339 | 135 |
| RIMBP3/NM_015672 | 136 |
| RN18S1/NR_003286 | 137 |
| RN5S1/NR_023363 | 138 |
| RN7SK/NR_001445 | 139 |
| RN7SL1/NR_002715 | 140 |
| RNF152/NM_173557 | 141 |
| RNF185/NM_001135825 | 142 |
| RNPS1/NM_001286625 | 143 |
| RNU11/NR_004407 | 144 |
| RNU1-4/NR_004421 | 145 |
| RNU2-1/NR_002716 | 146 |
| RNU4-2/NR_003137 | 147 |
| RNU5F-1/NR_002753 | 148 |
| RNU6-2/NR_125730 | 149 |
| RNY1/NR_004391 | 150 |
| RP11-165H20.1/NM_015629 | 151 |
| RPL21P68/NG_010409 | 152 |
| RPS27AP17/NG_011207 | 153 |
| S1PR3/NM_005226 | 154 |
| SART1/NM_005146 | 155 |
| SDAD1/NM_001288983 | 156 |
| SEPP1/NM_005410 | 157 |
| SETD9/NM_153706 | 158 |
| SIGLEC14/NM_001098612 | 159 |
| SKAP1/NM_003726 | 160 |
| SLC36A3/NM_001145017 | 161 |
| SLC8A1/NM_001112800 | 162 |
| SLC9B1P2/NG_009550 | 163 |
| SLCO4C1/NM_180991 | 164 |
| SMNDC1/NM_005871 | 165 |
| SNORA1/NR_003026 | 166 |
| SNORA72/NR_002581 | 167 |
| SNORA75/NR_002921 | 168 |
| SNORD114-2/NR_003194 | 169 |
| SNORD115-10/NR_003302 | 170 |
| SNORD116-19/NR_001290 | 171 |
| SNORD18A/NR_002441 | 172 |
| SNORD3B1/NR_003271 | 173 |
| SPOCK3/NM_001040159 | 174 |
| ST6GAL1/NM_173216 | 175 |
| STAMBPL1/NM_020799 | 176 |
| TAF1D/NM_024116 | 177 |
| TAS2R14/NM_023922 | 178 |
| TBCCD1/NM_001134415 | 179 |
| TCEB3B/NM_016427 | 180 |
| TMEM100/NM_001099640 | 181 |
| TMIGD1/NM_001099640 | 182 |
| ERBB2/NM_016272 | 183 |
| TUBB8/NM_177987 | 184 |
| UBD/NM_006398 | 185 |
| UGT2B17/NM_001077 | 186 |
| UQCRFS1/NM_006003 | 187 |
| VNN1/NM_004666 | 188 |
| ZC3HC1/NM_016478 | 189 |
| ZCCHC12/NM_173798 | 190 |
| ZNF222/NM_001129996 | 191 |
| ZNF259/NM_001129996 | 192 |
| ZNF439/NM_001348718 | 193 |
| ZNF487P/NR_026693 | 194 |
| ZNF569/NM_152484 | 195 |
| ZNF678/NM_178549 | 196 |
| ZNF684/NM_152373 | 197 |
| ZNF716/NM_001159279 | 198 |
| ZNF718/NM_001039127 | 199 |
| ZNRF3/NM_001206998 | 200 |
| ZSCAN29/NM_152455 | 201 |

Corresponding hypoxic biomarkers also include amino acid sequences that display substantial sequence similarity or identity to the amino acid sequence of a reference hypoxia biomarker polypeptide. In general, an amino acid sequence that corresponds to a reference amino acid sequence will display at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to a reference amino acid sequence selected from any one of the sequences listed in Table 6.

In some embodiments, calculations of sequence similarity or sequence identity between sequences are performed as follows:

To determine the percentage identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, usually at least 40%, more usually at least 50%, 60%, and even more usually at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percentage identity between the two sequences is a function of the number of identical amino acid residues shared by the sequences at individual positions, taking into account the number of gaps, and the length of each gap, which need to be introduced for optimal alignment of the two sequences. By contrast, the percentage similarity between the two sequences is a function of the number of identical and similar amino acid residues shared by the sequences at individual positions, taking into account the number of gaps, and the length of each gap, which need to be introduced for optimal alignment of the two sequences.

TABLE 6

| Peptide biomarker | SEQ ID NO: |
|---|---|
| ARNTL/O00327 | 202 |
| CD1C/P29017 | 203 |
| HHEX/Q03014 | 204 |
| KLRG1/Q96E93 | 205 |
| MMP16/P51512 | 206 |
| FGFR2/NP_000132 | 207 |
| GATA2/P23769 | 208 |
| CEACAM7/Q14002 | 209 |
| OGN/P20774 | 210 |
| AGTR1/P30556 | 211 |
| ARMCX1/Q9P291 | 212 |
| C10orf25/Q5T742 | 213 |
| C14orf169/NP_078920 | 214 |
| C18orf34/Q5BJE1 | 215 |
| C18orf62/Q3B7S5 | 216 |
| C1QTNF9/P0C862 | 217 |
| C2orf76/Q3KRA6 | 218 |
| C6orf165/Q8IYR0 | 219 |
| C7orf45/Q8WWF3 | 220 |
| C7orf62/Q8TBZ9 | 221 |
| C9orf131/Q5VYM1 | 222 |
| C9orf85/Q96MD7 | 223 |
| CA10/Q9NS85 | 224 |
| CAPNS2/Q96L46 | 225 |
| CCDC121/Q6ZUS5 | 226 |
| CCDC141/B8ZZB3 | 227 |
| CDH10/Q9Y6N8 | 228 |
| CDH11/P55287 | 229 |
| CEP170P1/XP_011542644 | 230 |
| CYTIP/O60759 | 231 |
| DBF4/P32325 | 232 |
| DDX56/Q9NY93 | 233 |
| DPPA2/Q7Z7J5 | 234 |
| DTX2/Q86UW9 | 235 |
| EIF4G2/P78344 | 236 |
| ELMO1/Q92556 | 237 |
| ELOVL3/Q9HB03 | 238 |
| ENAM/Q9NRM1 | 239 |
| FAM172BP/A6NC97 | 240 |
| FCGR1A/P12314 | 241 |
| FFAR2/O15552 | 242 |
| FGF12/O15552 | 243 |
| FLJ42102/Q6ZVU0 | 244 |
| FLJ44838/A6NFN3 | 245 |
| FLJ45721/Q6ZS92 | 246 |
| FN1/P02751 | 247 |
| FOXP2/O15409 | 248 |
| GDI2/P50395 | 249 |
| GGTLC2/Q14390 | 250 |
| GK2/Q14410 | 251 |
| GPR78/Q96P69 | 252 |
| HARBI1/Q96MB7 | 253 |
| HEMGN/Q9BXL5 | 254 |
| HIST1H2AA/Q96QV6 | 255 |
| HMGN2/P05204 | 256 |
| HOXC13/P31276 | 257 |
| HPDL/Q96IR7 | 258 |
| HRCT1/Q6UXD1 | 259 |
| HRH1/P35367 | 260 |
| HS3ST3B1/Q9Y662 | 261 |
| HSD3B2/P26439 | 262 |
| ICA1L/Q8NDH6 | 263 |
| IDO2/Q6ZQW0 | 264 |
| IL10/P22301 | 265 |
| IL17A/Q16552 | 266 |
| IL18/Q14116 | 267 |
| IL1B/P01584 | 268 |
| KARS/Q15046 | 269 |
| KBTBD6/Q86V97 | 270 |
| KCNA2/P16389 | 271 |
| KLB/Q86Z14 | 272 |
| KMO/015229 | 273 |
| KRT10/P13645 | 274 |
| KRT222/Q8N1A0 | 275 |
| LCN1/P31025 | 276 |
| LIG4/P49917 | 277 |
| LINC00312/Q9Y6C7 | 278 |
| LINC00596/Q86U02 | 279 |
| LLPH/Q9BRT6 | 280 |
| LRRC37A5P/Q49AS3 | 281 |
| LRRC53/A6NM62 | 282 |
| LTA/P01374 | 283 |
| MEIG1/Q5JSS6 | 284 |
| METTL2A/Q96IZ6 | 285 |
| MGAT2/Q96IZ6 | 286 |
| MILR1/Q7Z6M3 | 287 |
| MMP27/Q9H306 | 288 |
| MPP1/Q00013 | 289 |
| MRPL30/Q8TCC3 | 290 |
| MUTYH/Q9UIF7 | 291 |
| MYH8/P13535 | 292 |
| ND6/P03923 | 293 |
| NEFM/Q9UK51 | 294 |
| NEUROD1/Q13562 | 295 |
| NONO/Q15233 | 296 |
| NPCDR1/Q9BY65 | 297 |
| NPY5R/Q15761 | 298 |
| OCM/P0CE72 | 299 |
| OCM2/P0CE71 | 300 |
| OR10A2/Q9H208 | 301 |
| OR2A12/Q8NGT7 | 302 |
| OR2D3/Q8NGH3 | 303 |
| OR2M3/Q8NG83 | 304 |
| OR2T2/Q6IF00 | 305 |
| OR2T35/Q8NGX2 | 306 |
| OR4D10/Q8NGI6 | 307 |
| OR4D2/P58180 | 308 |
| OR5J2/Q8NH18 | 309 |
| OR5M10/Q6IEU7 | 310 |
| OR8H3/Q8N146 | 311 |
| OR9G1/Q8NH87 | 312 |
| ORM2/P19652 | 313 |
| OTOG/Q6ZRI0 | 314 |
| PAGE2B/Q5JRK9 | 315 |
| PLAG1/Q6DJT9 | 316 |
| PRL/P01236 | 317 |
| PRPF8/Q6P2Q9 | 318 |
| PSCA/D3DWI6 | 319 |
| PTPN20A/Q4JDL3 | 320 |
| RBM44/Q6ZP01 | 321 |
| RBMY1C/P0DJD4 | 322 |
| RBMY1F/Q15415 | 323 |
| RFK/Q969G6 | 324 |
| RIMBP3/Q9UFD9 | 325 |
| RNF152/Q8N8N0 | 326 |
| RNF185/Q96GF1 | 327 |
| RNPS1/Q15287 | 328 |
| RP11-165H20.1/Q8WWY3 | 329 |
| S1PR3/Q99500 | 330 |
| SART1/O43290 | 331 |
| SDAD1/Q9NVU7 | 332 |
| SEPP1/P49908 | 333 |
| SETD9/Q8NE22 | 334 |
| SIGLEC14/Q08ET2 | 335 |
| SKAP1/Q86WV1 | 336 |
| SLC36A3/Q495N2 | 337 |
| SLC8A1/P32418 | 338 |
| SLCO4C1/Q6ZQN7 | 339 |
| SMNDC1/O75940 | 340 |
| SPOCK3/Q9BQ16 | 341 |
| ST6GAL1/P15907 | 342 |
| STAMBPL1/Q96FJ0 | 343 |
| TAF1D/Q9H5J8 | 344 |
| TAS2R14/Q9NYV8 | 345 |
| TBCCD1/Q9NVR7 | 346 |
| TCEB3B/Q8IYF1 | 347 |
| TMEM100/Q9NV29 | 348 |
| TMIGD1/Q9NV29 | 349 |
| TOB2/Q14106 | 350 |
| TUBB8/Q3ZCM7 | 351 |
| UBD/O15205 | 352 |
| UGT2B17/O75795 | 353 |
| UQCRFS1/P47985 | 354 |
| VNN1/O95497 | 355 |
| ZC3HC1/Q86WB0 | 356 |
| ZCCHC12/Q6PEW1 | 357 |
| ZNF222/Q9UK12 | 358 |
| ZNF259/O7531 | 359 |
| ZNF439/Q8NDP4 | 360 |
| ZNF569/Q5MCW4 | 361 |
| ZNF678/Q5SXM1 | 362 |
| ZNF684/Q5T5D7 | 363 |
| ZNF716/A6NP11 | 364 |
| ZNF718/Q3SXZ3 | 365 |
| ZNRF3/Q9ULT6 | 366 |
| ZSCAN29/Q8IWY8 | 367 |

The comparison of sequences and determination of percentage identity or percentage similarity between sequences can be accomplished using a mathematical algorithm. In certain embodiments, the percentage identity or similarity between amino acid sequences is determined using the Needleman and Wünsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In specific embodiments, the percent identity between nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A non-limiting set of parameters (and the one that should be used unless otherwise specified) includes a Blossum 62 scoring matrix with a gap penalty of 12, a gap extend penalty of 4, and a frameshift gap penalty of 5.

In some embodiments, the percentage identity or similarity between amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J Mol Biol., 215: 403-10). BLAST nucleotide searches can be performed with the NBLAST program, score=100, wordlength=12 to obtain nucleotide sequences homologous to 53010 nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Corresponding hypoxia biomarker polynucleotides also include nucleic acid sequences that hybridize to reference hypoxia biomarker polynucleotides, or to their complements, under stringency conditions described below. As used herein, the term “hybridizes under low stringency, medium stringency, high stringency, or very high stringency conditions” describes conditions for hybridization and washing. “Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a corresponding hypoxia biomarker polynucleotide is one that hybridizes to a disclosed nucleotide sequence under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

2.1 Sample Preparation

Generally, a sample is processed prior to hypoxia biomarker detection or quantification. For example, nucleic acid and/or proteins may be extracted, isolated, and/or purified from a sample prior to analysis. Various DNA, mRNA, and/or protein extraction techniques are well known to those skilled in the art. Processing may include centrifugation, ultracentrifugation, ethanol precipitation, filtration, fractionation, resuspension, dilution, concentration, etc. In some embodiments, methods and systems provide analysis (e.g., quantification of RNA or protein biomarkers) from raw sample (e.g., biological fluid such as blood, serum, etc.) without or with limited processing.

Methods may comprise steps of homogenizing a sample in a suitable buffer, removal of contaminants and/or assay inhibitors, adding a hypoxia biomarker capture reagent (e.g., a magnetic bead to which is linked an oligonucleotide complementary to a target hypoxia nucleic acid biomarker), incubated under conditions that promote the association (e.g., by hybridization) of the target biomarker with the capture reagent to produce a target biomarker:capture reagent complex, incubating the target biomarker:capture complex under target biomarker-release conditions. In some embodiments, multiple hypoxia biomarkers are isolated in each round of isolation by adding multiple hypoxia biomarkers capture reagents (e.g., specific to the desired biomarkers) to the solution. For example, multiple hypoxia biomarker capture reagents, each comprising an oligonucleotide specific for a different target hypoxia biomarker can be added to the sample for isolation of multiple hypoxia biomarker. It is contemplated that the methods encompass multiple experimental designs that vary both in the number of capture steps and in the number of target hypoxia biomarker captured in each capture step. In some embodiments, capture reagents are molecules, moieties, substances, or compositions that preferentially (e.g., specifically and selectively) interact with a particular biomarker sought to be isolated, purified, detected, and/or quantified. Any capture reagent having desired binding affinity and/or specificity to the particular hypoxia biomarker can be used in the present technology.

For example, the capture reagent can be a macromolecule such as a peptide, a protein (e.g., an antibody or receptor), an oligonucleotide, a nucleic acid (e.g., nucleic acids capable of hybridizing with the hypoxia biomarkers), vitamins, oligosaccharides, carbohydrates, lipids, or small molecules, or a complex thereof. As illustrative and non-limiting examples, an avidin target capture reagent may be used to isolate and purify targets comprising a biotin moiety, an antibody may be used to isolate and purify targets comprising the appropriate antigen or epitope, and an oligonucleotide may be used to isolate and purify a complementary oligonucleotide.

Any nucleic acids, including single-stranded and double-stranded nucleic acids, that are capable of binding, or specifically binding, to a target hypoxia biomarker can be used as the capture reagent. Examples of such nucleic acids include DNA, RNA, aptamers, peptide nucleic acids, and other modifications to the sugar, phosphate, or nucleoside base. Thus, there are many strategies for capturing a target and accordingly many types of capture reagents are known to those in the art.

In addition, hypoxia biomarker capture reagents may comprise a functionality to localize, concentrate, aggregate, etc. the capture reagent and thus provide a way to isolate and purify the target hypoxia biomarker when captured (e.g., bound, hybridized, etc.) to the capture reagent (e.g., when a target:capture reagent complex is formed). For example, in some embodiments the portion of the capture reagent that interacts with the hypoxia biomarker (e.g., an oligonucleotide) is linked to a solid support (e.g., a bead, surface, resin, column, and the like) that allows manipulation by the user on a macroscopic scale. Often, the solid support allows the use of a mechanical means to isolate and purify the target:capture reagent complex from a heterogeneous solution. For example, when linked to a bead, separation is achieved by removing the bead from the heterogeneous solution, e.g., by physical movement. In embodiments in which the bead is magnetic or paramagnetic, a magnetic field is used to achieve physical separation of the capture reagent (and thus the target hypoxia biomarker) from the heterogeneous solution.

The hypoxia biomarkers may be quantified or detected using any suitable technique. In specific embodiments, the hypoxia biomarkers are quantified using reagents that determine the level, abundance or amount of individual hypoxia biomarkers. Non-limiting reagents of this type include reagents for use in nucleic acid- and protein-based assays.

2.2 Quantification or Detection of Nucleic Acid Biomarkers

In illustrative nucleic acid-based assays, nucleic acid is isolated from cells contained in the biological sample according to standard methodologies (Sambrook, et al., 1989, supra; and Ausubel et al., 1994, supra). The nucleic acid is typically fractionated (e.g., poly A⁺ RNA) or whole cell RNA. Where RNA is used as the subject of detection, it may be desired to convert the RNA to a complementary DNA. In some embodiments, the nucleic acid is amplified by a template-dependent nucleic acid amplification technique. A number of template dependent processes are available to amplify the hypoxia biomarker sequences present in a given template sample. An exemplary nucleic acid amplification technique is the polymerase chain reaction (referred to as PCR), which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). Briefly, in PCR, two primer sequences are prepared that are complementary to regions on opposite complementary strands of the biomarker sequence. An excess of deoxynucleotide triphosphates are added to a reaction mixture along with a DNA polymerase, e.g., Taq polymerase. If a cognate hypoxia biomarker sequence is present in a sample, the primers will bind to the biomarker and the polymerase will cause the primers to be extended along the biomarker sequence by adding on nucleotides. By raising and lowering the temperature of the reaction mixture, the extended primers will dissociate from the biomarker to form reaction products, excess primers will bind to the biomarker and to the reaction products and the process is repeated. A reverse transcriptase PCR amplification procedure may be performed in order to quantify the amount of mRNA amplified. Methods of reverse transcribing RNA into cDNA are well known and described in Sambrook et al., 1989, supra. Alternative methods for reverse transcription utilize thermostable, RNA-dependent DNA polymerases. These methods are described in WO 90/07641. Polymerase chain reaction methodologies are well known in the art. In specific embodiments in which whole cell RNA is used, cDNA synthesis using whole cell RNA as a sample produces whole cell cDNA.

In certain advantageous embodiments, the template-dependent amplification involves quantification of transcripts in real-time. For example, RNA or DNA may be quantified using the real-time PCR technique (Higuchi, 1992, et al., Biotechnology 10: 413-417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR products and the relative mRNA abundance is only true in the linear range of the PCR reaction. The final concentration of the target DNA in the plateau portion of the curve is determined by the availability of reagents in the reaction mix and is independent of the original concentration of target DNA. In specific embodiments, multiplexed, tandem PCR (MT-PCR) is employed, which uses a two-step process for gene expression profiling from small quantities of RNA or DNA, as described for example in U.S. Pat. Appl. Pub. No. 20070190540. In the first step, RNA is converted into cDNA and amplified using multiplexed gene specific primers. In the second step each individual gene is quantitated by real time PCR. Real-time PCR is typically performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments of RT-PCR assays, a TAQMAN® probe is used for quantitating nucleic acid. Such assays may use energy transfer (“ET”), such as fluorescence resonance energy transfer (“FRET”), to detect and quantitate the synthesized PCR product. Typically, the TAQMAN® probe comprises a fluorescent label (e.g., a fluorescent dye) coupled to one end (e.g., the 5′-end) and a quencher molecule is coupled to the other end (e.g., the 3′-end), such that the fluorescent label and the quencher are in close proximity, allowing the quencher to suppress the fluorescence signal of the dye via FRET. When a polymerase replicates the chimeric amplicon template to which the fluorescent labeled probe is bound, the 5′-nuclease of the polymerase cleaves the probe, decoupling the fluorescent label and the quencher so that label signal (such as fluorescence) is detected. Signal (such as fluorescence) increases with each PCR cycle proportionally to the amount of probe that is cleaved.

TAQMAN® probes typically comprise a region of contiguous nucleotides having a sequence that is identically present in or complementary to a region of a hypoxia biomarker polynucleotide such that the probe is specifically hybridizable to the resulting PCR amplicon. In some embodiments, the probe comprises a region of at least 6 contiguous nucleotides having a sequence that is fully complementary to or identically present in a region of a target hypoxia biomarker polynucleotide, such as comprising a region of at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a target hypoxia biomarker polynucleotide to be detected and/or quantitated.

In addition to the TAQMAN® assays, other real-time PCR chemistries useful for detecting PCR products in the methods presented herein include, but are not limited to, Molecular Beacons, Scorpion probes and intercalating dyes, such as SYBR Green, EvaGreen, thiazole orange, YO-PRO, TO-PRO, etc. For example, Molecular Beacons, like TAQMAN® probes, use FRET to detect and quantitate a PCR product via a probe having a fluorescent label (e.g., a fluorescent dye) and a quencher attached at the ends of the probe. Unlike TAQMAN® probes, however, Molecular Beacons remain intact during the PCR cycles. Molecular Beacon probes form a stem-loop structure when free in solution, thereby allowing the fluorescent label and quencher to be in close enough proximity to cause fluorescence quenching. When the Molecular Beacon hybridizes to a target, the stem-loop structure is abolished so that the fluorescent label and the quencher become separated in space and the fluorescent label fluoresces. Molecular Beacons are available, e.g., from Gene Link™ (see, genelink.com/newsite/products/mbintro.asp).

In some embodiments, Scorpion probes can be used as both sequence-specific primers and for PCR product detection and quantitation. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent label (e.g., a fluorescent dye molecule) is attached to the 5′-end of the Scorpion probe, and a quencher is attached to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the fluorescent label on the 5′-end to fluoresce and generate a signal. Scorpion probes are available from, e.g., Premier Biosoft International (see, www.premierbiosoft.com/tech_notes/Scorpion.html).

In some embodiments, labels that can be used on the FRET probes include colorimetric and fluorescent dyes such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some embodiments, the same quencher may be used for multiple dyes, for example, a broad spectrum quencher, such as an Iowa Black® quencher (Integrated DNA Technologies, Coralville, Iowa) or a Black Hole Quencher™ (BHQ™; Sigma-Aldrich, St. Louis, Mo.).

In some embodiments, for example, in a multiplex reaction in which two or more moieties (such as amplicons) are detected simultaneously, each probe comprises a detectably different dye such that the dyes may be distinguished when detected simultaneously in the same reaction. One skilled in the art can select a set of detectably different dyes for use in a multiplex reaction. In some embodiments, multiple target hypoxia biomarker polynucleotides are detected and/or quantitated in a single multiplex reaction. In some embodiments, each probe that is targeted to a different hypoxia biomarker polynucleotide is spectrally distinguishable when released from the probe. Thus, each target hypoxia biomarker polynucleotide is detected by a unique fluorescence signal.

Specific examples of fluorescently labeled ribonucleotides useful in the preparation of real-time PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of real-time PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In certain embodiments, target nucleic acids are quantified using blotting techniques, which are well known to those of skill in the art. Southern blotting involves the use of DNA as a target, whereas Northern blotting involves the use of RNA as a target. Each provides different types of information, although cDNA blotting is analogous, in many aspects, to blotting or RNA species. Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above. Following detection/quantification, one may compare the results seen in a given subject with a control reaction or a statistically significant reference group or population of control subjects as defined herein. In this way, it is possible to correlate the amount of hypoxia biomarker nucleic acid detected with the progression or severity of the disease.

Also contemplated are biochip-based technologies such as those described by Hacia et al. (1996, Nature Genetics 14: 441-447) and Shoemaker et al. (1996, Nature Genetics 14: 450-456). Briefly, these techniques involve quantitative methods for analyzing large numbers of genes rapidly and accurately. By tagging genes with oligonucleotides or using fixed nucleic acid probe arrays, one can employ biochip technology to segregate target molecules as high-density arrays and screen these molecules on the basis of hybridization. See also Pease et al. (1994, Proc. Natl. Acad. Sci. U.S.A. 91: 5022-5026); Fodor et al. (1991, Science 251: 767-773). Briefly, nucleic acid probes to hypoxia biomarker polynucleotides are made and attached to biochips to be used in screening and diagnostic methods, as outlined herein. The nucleic acid probes attached to the biochip are designed to be substantially complementary to specific expressed hypoxia biomarker nucleic acids, i.e., the target sequence (either the target sequence of the sample or to other probe sequences, for example in sandwich assays), such that hybridization of the target sequence and the probes of the present invention occur. This complementarity need not be perfect; there may be any number of base pair mismatches, which will interfere with hybridization between the target sequence and the nucleic acid probes of the present invention. However, if the number of mismatches is so great that no hybridization can occur under even the least stringent of hybridization conditions, the sequence is not a complementary target sequence. In certain embodiments, more than one probe per sequence is used, with either overlapping probes or probes to different sections of the target being used. That is, two, three, four or more probes, with three being desirable, are used to build in a redundancy for a particular target. The probes can be overlapping (i.e., have some sequence in common), or separate.

In an illustrative biochip analysis, oligonucleotide probes on the biochip are exposed to or contacted with a nucleic acid sample suspected of containing one or more hypoxia biomarker polynucleotides under conditions favoring specific hybridization. Sample extracts of DNA or RNA, either single or double-stranded, may be prepared from fluid suspensions of biological materials, or by grinding biological materials, or following a cell lysis step which includes, but is not limited to, lysis effected by treatment with SDS (or other detergents), osmotic shock, guanidinium isothiocyanate and lysozyme. Suitable DNA, which may be used in the method of the invention, includes cDNA. Such DNA may be prepared by any one of a number of commonly used protocols as for example described in Ausubel, et al., 1994, supra, and Sambrook, et al., 1989, supra.

Suitable RNA, which may be used in the method of the invention, includes messenger RNA, complementary RNA transcribed from DNA (cRNA) or genomic or subgenomic RNA. Such RNA may be prepared using standard protocols as for example described in the relevant sections of Ausubel, et al. 1994, supra and Sambrook, et al. 1989, supra).

cDNA may be fragmented, for example, by sonication or by treatment with restriction endonucleases. Suitably, cDNA is fragmented such that resultant DNA fragments are of a length greater than the length of the immobilized oligonucleotide probe(s) but small enough to allow rapid access thereto under suitable hybridization conditions. Alternatively, fragments of cDNA may be selected and amplified using a suitable nucleotide amplification technique, as described for example above, involving appropriate random or specific primers.

Usually the target hypoxia biomarker polynucleotides are detectably labeled so that their hybridization to individual probes can be determined. The target polynucleotides are typically detectably labeled with a reporter molecule illustrative examples of which include chromogens, catalysts, enzymes, fluorochromes, chemiluminescent molecules, bioluminescent molecules, lanthanide ions (e.g., Eu³⁴), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like. Illustrative labels of this type include large colloids, for example, metal colloids such as those from gold, selenium, silver, tin and titanium oxide. In some embodiments in which an enzyme is used as a direct visual label, biotinylated bases are incorporated into a target polynucleotide.

The hybrid-forming step can be performed under suitable conditions for hybridizing oligonucleotide probes to test nucleic acid including DNA or RNA. In this regard, reference may be made, for example, to NUCLEIC ACID HYBRIDIZATION, A PRACTICAL APPROACH (Homes and Higgins, eds.) (IRL press, Washington D.C., 1985). In general, whether hybridization takes place is influenced by the length of the oligonucleotide probe and the polynucleotide sequence under test, the pH, the temperature, the concentration of mono- and divalent cations, the proportion of G and C nucleotides in the hybrid-forming region, the viscosity of the medium and the possible presence of denaturants. Such variables also influence the time required for hybridization. The preferred conditions will therefore depend upon the particular application. Such empirical conditions, however, can be routinely determined without undue experimentation.

After the hybrid-forming step, the probes are washed to remove any unbound nucleic acid with a hybridization buffer. This washing step leaves only bound target polynucleotides. The probes are then examined to identify which probes have hybridized to a target polynucleotide.

The hybridization reactions are then detected to determine which of the probes has hybridized to a corresponding target sequence. Depending on the nature of the reporter molecule associated with a target polynucleotide, a signal may be instrumentally detected by irradiating a fluorescent label with light and detecting fluorescence in a fluorimeter; by providing for an enzyme system to produce a dye which could be detected using a spectrophotometer; or detection of a dye particle or a colored colloidal metallic or non-metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focused beam or laser light. In such a case, a charge couple device (CCD) or a photocell can be used to scan for emission of light from a probe:target polynucleotide hybrid from each location in the micro-array and record the data directly in a digital computer. In some cases, electronic detection of the signal may not be necessary. For example, with enzymatically generated color spots associated with nucleic acid array format, visual examination of the array will allow interpretation of the pattern on the array. In the case of a nucleic acid array, the detection means is suitably interfaced with pattern recognition software to convert the pattern of signals from the array into a plain language genetic profile. In certain embodiments, oligonucleotide probes specific for different hypoxia biomarker polynucleotides are in the form of a nucleic acid array and detection of a signal generated from a reporter molecule on the array is performed using a “chip reader”. A detection system that can be used by a “chip reader” is described for example by Pirrung et al. (U.S. Pat. No. 5,143,854). The chip reader will typically also incorporate some signal processing to determine whether the signal at a particular array position or feature is a true positive or maybe a spurious signal. Exemplary chip readers are described for example by Fodor et al. (U.S. Pat. No. 5,925,525). Alternatively, when the array is made using a mixture of individually addressable kinds of labeled microbeads, the reaction may be detected using flow cytometry.

In certain embodiments, the hypoxia biomarker is a target RNA (e.g., mRNA) or a DNA copy of the target RNA whose level or abundance is measured using at least one nucleic acid probe that hybridizes under at least low, medium, or high stringency conditions to the target RNA or to the DNA copy, wherein the nucleic acid probe comprises at least 15 (e.g., 15, 16, 17, 18, 19, 20, 21, 22, 23, 24, 25, 26, 27, 28, 29, 30, or more) contiguous nucleotides of hypoxia biomarker polynucleotide. In some embodiments, the measured level or abundance of the target RNA or its DNA copy is normalized to the level or abundance of a reference RNA or a DNA copy of the reference RNA. Suitably, the nucleic acid probe is immobilized on a solid or semi-solid support. In illustrative examples of this type, the nucleic acid probe forms part of a spatial array of nucleic acid probes. In some embodiments, the level of nucleic acid probe that is bound to the target RNA or to the DNA copy is measured by hybridization (e.g., using a nucleic acid array). In other embodiments, the level of nucleic acid probe that is bound to the target RNA or to the DNA copy is measured by nucleic acid amplification (e.g., using a polymerase chain reaction (PCR)). In still other embodiments, the level of nucleic acid probe that is bound to the target RNA or to the DNA copy is measured by nuclease protection assay.

Sequencing technologies such as Sanger sequencing, pyrosequencing, sequencing by ligation, massively parallel sequencing, also called “Next-generation sequencing” (NGS), and other high-throughput sequencing approaches with or without sequence amplification of the target can also be used to detect or quantify the presence of hypoxia nucleic acid biomarker in a sample. Sequence-based methods can provide further information regarding alternative splicing and sequence variation in previously identified genes. Sequencing technologies include a number of steps that are grouped broadly as template preparation, sequencing, detection and data analysis. Current methods for template preparation involve randomly breaking genomic DNA into smaller sizes from which each fragment is immobilized to a support. The immobilization of spatially separated fragment allows thousands to billions of sequencing reaction to be performed simultaneously. A sequencing step may use any of a variety of methods that are commonly known in the art. One specific example of a sequencing step uses the addition of nucleotides to the complementary strand to provide the DNA sequence. The detection steps range from measuring bioluminescent signal of a synthesized fragment to four-color imaging of single molecule. In some embodiments in which NGS is used to detect or quantify the presence of a hypoxia nucleic acid biomarker in a sample, the methods are suitably selected from semiconductor sequencing (Ion Torrent; Personal Genome Machine); Helicos True Single Molecule Sequencing (tSMS) (Harris et al. 2008, Science 320:106-109); 454 sequencing (Roche) (Margulies et al. 2005, Nature, 437, 376-380); SOLiD technology (Applied Biosystems); SOLEXA sequencing (Illumina); single molecule, real-time (SMRT™) technology of Pacific Biosciences; nanopore sequencing (Soni and Meller, 2007. Clin Chem 53: 1996-2001); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys).

2.3 Quantification or Detection of Protein Biomarkers

In other embodiments, hypoxia biomarker protein levels are assayed using protein-based assays known in the art. For example, when the hypoxia biomarker protein is an enzyme, the protein can be quantified based upon its catalytic activity or based upon the number of molecules of the protein contained in a sample. Antibody-based techniques may be employed including, for example, immunoassays, such as the enzyme-linked immunosorbent assay (ELISA) and the radioimmunoassay (RIA). For example, the anti-G9A antibody ab133482 (abcam) could be used to measure the group 2 hypoxia biomarker G9a.

In specific embodiments, protein-capture arrays that permit simultaneous detection and/or quantification of a large number of proteins are employed. For example, low-density protein arrays on filter membranes, such as the universal protein array system (Ge, 2000 Nucleic Acids Res. 28(2): e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in subjects pre- and post-drug treatment.

Exemplary protein capture arrays include arrays comprising spatially addressed antigen-binding molecules, commonly referred to as antibody arrays, which can facilitate extensive parallel analysis of numerous proteins defining a proteome or subproteome. Antibody arrays have been shown to have the required properties of specificity and acceptable background, and some are available commercially (e.g., BD Biosciences, Clontech, Bio-Rad and Sigma). Various methods for the preparation of antibody arrays have been reported (see, e.g., Lopez et al., 2003 J. Chromatogram. B 787:19-27; Cahill, 2000 Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210). The antigen-binding molecules of such arrays may recognize at least a subset of proteins expressed by a cell or population of cells, illustrative examples of which include growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors and cell-surface antigens.

Individual spatially distinct protein-capture agents are typically attached to a support surface, which is generally planar or contoured. Common physical supports include glass slides, silicon, microwells, nitrocellulose or PVDF membranes, and magnetic and other microbeads.

Particles in suspension can also be used as the basis of arrays, providing they are coded for identification; systems include color coding for microbeads (e.g., available from Luminex, Bio-Rad and Nanomics Biosystems) and semiconductor nanocrystals (e.g., QDOTS™, available from Quantum Dots), and barcoding for beads (ULTRAPLEX™, available from Smartbeads) and multimetal microrods (NANOBARCODES™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtiter plate or in separate test tubes.

In operation, a protein sample, which is optionally fragmented to form peptide fragments (see, e.g., U.S. Pat. App. Pub. 2002/0055186), is delivered to a protein-capture array under conditions suitable for protein or peptide binding, and the array is washed to remove unbound or non-specifically bound components of the sample from the array. Next, the presence or amount of protein or peptide bound to each feature of the array is detected using a suitable detection system. The amount of protein bound to a feature of the array may be determined relative to the amount of a second protein bound to a second feature of the array. In certain embodiments, the amount of the second protein in the sample is already known or known to be invariant.

In specific embodiments, the hypoxia biomarker is a target polypeptide whose level is measured using at least one antigen-binding molecule that is immuno-interactive with the target polypeptide. In these embodiments, the measured level of the target polypeptide is normalized to the level of a reference polypeptide. Suitably, the antigen-binding molecule is immobilized on a solid or semi-solid support. In illustrative examples of this type, the antigen-binding molecule forms part of a spatial array of antigen-binding molecule. In some embodiments, the level of antigen-binding molecule that is bound to the target polypeptide is measured by immunoassay (e.g., using an ELISA).

2.4 Diseases and Conditions Associated with Hypoxia

The methods described above and elsewhere herein are suitable for determining the likelihood of any disease or condition that is known to occur in hypoxic environments. By way of a non-limiting example, the suitable diseases or conditions include cancer, ischemic stroke, arthritis (e.g., rheumatoid arthritis), inflammation, cartilage erosion, abnormal energy metabolism, oxidative damage, etc.

In some embodiments, the hypoxic condition is diagnosed in a cancer or tumor.

In certain embodiments of this type, the cancer is a solid tumor. In some embodiments of this type, the cancer is a blood tumor (i.e., not a solid tumor). The type of cancer includes, but is not limited to, one or more of the cancer types such as primary cancer, metastatic cancer, breast cancer, colon cancer, rectal cancer, lung cancer, oropharyngeal cancer, hypopharyngeal cancer, oesophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningeal cancer, astrocytoma, glioma, glioblastoma, retinoblastoma, neuroma, neuroblastoma, Schwannoma, meningioma, solid tumors arising from hematopoietic malignancies, leukaemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer, melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, refractory malignancy, advanced metastatic cancer, solid tumor, metastatic melanoma, prostate cancer, solid tumors, recurrent or persistent ovarian epithelial cancer, fallopian tube cancer, lung cancer, and primary peritoneal cancer.

2.5 Diseases and Conditions Associated with Aberrant G9a Expression

The biomarkers disclosed above and elsewhere herein also have utility for determining the likelihood of the presence or absence of a G9a-associated disease or condition in a subject. Although aberrant G9a expression is shown herein to be indicative of a hypoxic condition, other conditions that occurring in normoxic environments that have increased G9a polypeptide relative to a healthy condition are considered to be suitable for diagnosing with the biomarkers disclosed above and elsewhere herein.

For example, diseases and conditions that are associated with aberrant G9a expression include choline deficiency disease and borderline glaucoma.

Accordingly, the present invention also provides methods for determining an indicator used in assessing a likelihood of the presence or absence of a G9a-associated disease or condition in a subject, the method comprising, consisting or consisting essentially of: (1) determining a biomarker value that is measured or derived for at least one G9a-associated biomarker (e.g., 1, 2, 3, 4, 5, 6, 7, 8, 9 or 10 G9a-associated biomarkers) in a sample obtained from the subject, wherein the at least one G9a-associated biomarker is selected from ARNTL, CD1C, HHEX, KLRG1, MMP16, FGFR2, GATA2, CEACAM7, OGN, and AGTR1; and (2) determining the indicator using the biomarker value(s), wherein the indicator is at least partially indicative of the likelihood of the presence or absence of the G9a-associated condition in the subject.

2.6 Kits

All the essential reagents required for detecting and quantifying the hypoxia biomarkers of the invention may be assembled together in a kit. In some embodiments, the kit comprises a reagent that permits quantification of at least one hypoxia biomarker. In some embodiments the kit comprises: (i) a reagent that allows quantification (e.g., determining the level or abundance) of at least one hypoxia biomarker. In some embodiments, the kit further comprises (ii) a reagent that allows quantification (e.g., determining the level or abundance) of a second hypoxia biomarker, wherein the second hypoxia biomarker is G9a.

In the context of the present invention, “kit” is understood to mean a product containing the different reagents necessary for carrying out the methods of the invention packed so as to allow their transport and storage. Materials suitable for packing the components of the kit include crystal, plastic (polyethylene, polypropylene, polycarbonate and the like), bottles, vials, paper, envelopes and the like. Additionally, the kits of the invention can contain instructions for the simultaneous, sequential or separate use of the different components contained in the kit. The instructions can be in the form of printed material or in the form of an electronic support capable of storing instructions such that they can be read by a subject, such as electronic storage media (magnetic disks, tapes and the like), optical media (CD-ROM, DVD) and the like. Alternatively, or in addition, the media can contain internet addresses that provide the instructions.

Reagents that allow quantification of a hypoxia biomarker include compounds or materials, or sets of compounds or materials, which allow quantification of the hypoxia biomarker. In specific embodiments, the compounds, materials or sets of compounds or materials permit determining the expression level of a gene (e.g., hypoxia biomarker gene), including without limitation the extraction of RNA material, the determination of the level of a corresponding RNA, etc., primers for the synthesis of a corresponding cDNA, primers for amplification of DNA, and/or probes capable of specifically hybridizing with the RNAs (or the corresponding cDNAs) encoded by the genes, TaqMan probes, etc.

The kits may also optionally include appropriate reagents for detection of labels, positive and negative controls, washing solutions, blotting membranes, microtiter plates, dilution buffers and the like. For example, a nucleic acid-based detection kit may include (i) a hypoxia biomarker polynucleotide (which may be used as a positive control); and (ii) a primer or probe that specifically hybridizes to a hypoxia biomarker polynucleotide. Also included may be enzymes suitable for amplifying nucleic acids including various polymerases (reverse transcriptase, Taq, SEQUENASE™, DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) a hypoxia biomarker polypeptide (which may be used as a positive control); and (ii) an antibody that binds specifically to a hypoxia biomarker polypeptide. The kit can also feature various devices (e.g., one or more) and reagents (e.g., one or more) for performing one of the assays described herein; and/or printed instructions for using the kit to quantify the expression of a hypoxia biomarker gene.

The reagents described herein, which may be optionally associated with detectable labels, can be presented in the format of a microfluidics card, a chip or chamber, a microarray or a kit adapted for use with the assays described in the examples or below, e.g., RT-PCR or Q PCR techniques described herein.

The reagents also have utility in compositions for detecting and quantifying the biomarkers of the invention. For example, a reverse transcriptase may be used to reverse transcribe RNA transcripts, including mRNA, in a nucleic acid sample, to produce reverse transcribed transcripts, including reverse transcribed mRNA (also referred to as “cDNA”). In specific embodiments, the reverse transcribed mRNA is whole cell reverse transcribed mRNA (also referred to herein as “whole cell cDNA”). The nucleic acid sample is suitably derived from components of the immune system, representative examples of which include components of the innate and adaptive immune systems as broadly discussed for example above. In specific embodiments, the reverse transcribed RNA is derived blood cells (e.g., peripheral blood cells). Suitably, the reverse transcribed RNA is derived leukocytes.

The reagents are suitably used to quantify the reverse transcribed transcripts. For example, oligonucleotide primers that hybridize to the reverse transcribed transcript can be used to amplify at least a portion of the reverse transcribed transcript via a suitable nucleic acid amplification technique, e.g., RT-PCR or qPCR techniques described herein. Alternatively, oligonucleotide probes may be used to hybridize to the reverse transcribed transcript for the quantification, using a nucleic acid hybridization analysis technique (e.g., microarray analysis), as described for example above. Thus, in some embodiments, a respective oligonucleotide primer or probe is hybridized to a complementary nucleic acid sequence of a reverse transcribed transcript in the compositions of the invention. The compositions typically comprise labeled reagents for detecting and/or quantifying the reverse transcribed transcripts. Representative reagents of this type include labeled oligonucleotide primers or probes that hybridize to RNA transcripts or reverse transcribed RNA, labeled RNA, labeled reverse transcribed RNA as well as labeled oligonucleotide linkers or tags (e.g., a labeled RNA or DNA linker or tag) for labeling (e.g., end labeling such as 3′ end labeling) RNA or reverse transcribed RNA. The primers, probes, RNA or reverse transcribed RNA (i.e., cDNA) (whether labeled or non-labeled) may be immobilized or free in solution. Representative reagents of this type include labeled oligonucleotide primers or probes that hybridize to reverse transcribed and transcripts as well as labeled reverse transcribed transcripts. The label can be any reporter molecule as known in the art, illustrative examples of which are described above and elsewhere herein.

The present invention also encompasses non-reverse transcribed RNA embodiments in which cDNA is not made and the RNA transcripts are directly the subject of the analysis. Thus, in other embodiments, reagents are suitably used to quantify RNA transcripts directly. For example, oligonucleotide probes can be used to hybridize to transcripts for quantification of hypoxia biomarkers of the invention, using a nucleic acid hybridization analysis technique (e.g., microarray analysis), as described for example above. Thus, in some embodiments, a respective oligonucleotide probe is hybridized to a complementary nucleic acid sequence of a hypoxia biomarker transcript in the compositions of the invention. In illustrative examples of this type, the compositions may comprise labeled reagents that hybridize to transcripts for detecting and/or quantifying the transcripts. Representative reagents of this type include labeled oligonucleotide probes that hybridize to transcripts as well as labeled transcripts. The primers or probes may be immobilized or free in solution.

2.7 Methods of Managing Therapy

The present invention also extends to the management of a disease or condition that is associated with hypoxia (e.g., a hypoxic cancer), or prevention of further progression of the disease or condition, or assessment of the efficacy of therapies in subjects following positive diagnosis for the presence of a hypoxic condition, in a subject. Once a subject is positively identified as having a hypoxic condition, the subject may be administered a therapeutic agent for treating the hypoxic condition such as a G9a antagonist, illustrative examples of which are described in the International PCT Patent Publication No. WO2015/200329 and U.S. Patent Publication No. 2015/0274660, which are incorporated herein by reference in their entirety.

Other examples of G9a antagonists that may be suitable for use with the present invention include chaetocin, BIX-01294, UNC0224, UNC0638, UNC0642, UNC0646, and A-366.

Typically, the therapeutic agents will be administered in pharmaceutical (or veterinary) compositions together with a pharmaceutically acceptable carrier and in an effective amount to achieve their intended purpose. The dose of active compounds administered to a subject should be sufficient to achieve a beneficial response in the subject over time such as a reduction in, or relief from, hypoxia. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner. In determining the effective amount of the active compound(s) to be administered in the treatment or prevention of a hypoxic condition, the medical practitioner or veterinarian may evaluate severity of any symptom or clinical sign associated with the presence of the hypoxic. In any event, those of skill in the art may readily determine suitable dosages of the therapeutic agents and suitable treatment regimens without undue experimentation.

The therapeutic agents may be administered in concert with adjunctive (palliative) therapies to increase oxygen supply to major organs, increase blood flow to major organs and/or to reduce the inflammatory response. Illustrative examples of such adjunctive therapies include non-steroidal-anti-inflammatory drugs (NSAIDs), intravenous saline and oxygen.

The present invention also contemplates the use of the indicator-determining methods, apparatus, compositions and kits disclosed herein in methods of treating, preventing or inhibiting the development of a hypoxic condition (e.g., a hypoxic cancer) in a subject. These methods (also referred to herein as “treatment methods”) generally comprise: exposing the subject to a treatment regimen for treating a hypoxic condition, or avoiding exposing the subject to a treatment regimen for treating a disease or condition that is not associated with hypoxia, based on an indicator obtained from an indicator-determining method as disclosed herein. In specific embodiments, the treatment methods comprise: (a) determining a plurality of biomarker values for at least one (e.g., 1, 2, 3, 4, 5, 6, 7, 8, 9, 10, or more) hypoxia biomarkers of the subject, each biomarker value being indicative of a value measured or derived for a respective hypoxia biomarker; (b) determining an indicator using a combination of the plurality of biomarker values, the indicator being at least partially indicative of the presence or absence of a hypoxic condition (e.g., a hypoxic cancer), wherein: (i) at least one (e.g., 1, 2, 3, 4, 5, 6, 7, 8, 9, 10, or more) hypoxia biomarkers; and (ii) the indicator has a performance value greater than or equal to a performance threshold representing the ability of the indicator to diagnose the presence or absence of hypoxia; and (c) administering to the subject, on the basis that the indicator indicates the presence of a hypoxic condition, an effective amount of an agent that treats or ameliorates the symptoms or reverses or inhibits the development of the hypoxic condition.

In advantageous embodiments, the treatment methods comprise: (1) determining a plurality of measured biomarker values, each measured biomarker value being a measured value of an individual hypoxia biomarker of the subject; and (2) applying a function to at least two of the measured biomarker values to determine at least one derived biomarker value, the at least one derived biomarker value being indicative of a value of a corresponding derived hypoxia biomarker. The function suitably includes at least one of: (a) multiplying two biomarker values; (b) dividing two biomarker values; (c) adding two biomarker values; (d) subtracting two biomarker values; (e) a weighted sum of at least two biomarker values; (f) a log sum of at least two biomarker values; (g) a geometric mean of at least two biomarker values; and (h) a sigmoidal function of at least two biomarker values.

The present invention can be practiced in the field of predictive medicine for the purpose of diagnosis or monitoring the presence or development of hypoxic condition in a subject, and/or monitoring response to therapy efficacy. The biomarker profiles and corresponding indicators of the present invention further enable determination of endpoints in pharmacotranslational studies. For example, clinical trials can take many months or even years to establish the pharmacological parameters for a medicament to be used in treating or preventing a hypoxic condition (e.g., hypoxic cancer). However, these parameters may be associated with a biomarker profile and corresponding indicator of a health state (e.g., a healthy condition). Hence, the clinical trial can be expedited by selecting a treatment regimen (e.g., medicament and pharmaceutical parameters), which results in a biomarker profile associated with a desired health state (e.g., healthy condition). This may be determined for example by: (1) determining biomarker values that are measured or derived for at least two (e.g., 2, 3, 4, 5, 6, 7, 8, 9, 10, or more) corresponding hypoxia biomarker of a subject after treatment with a treatment regimen; (2) determining the indicator using the biomarker values; and (3) determining that the treatment regimen is effective for changing the health status of the subject to the desired health state (e.g., healthy condition) on the basis that the indicator indicates the presence of a healthy condition or the presence of a condition of a lower degree relative to the degree of the condition in the subject before treatment with the treatment regimen. As used herein, the term “degree” refers to the extent or stage of a condition. Alternatively, selection of the treatment regimen may be determined by: (a) determining a plurality of biomarker values for at least two (e.g., 2, 3, 4, 5, 6, 7, 8, 9, 10, or more) hypoxia biomarkers of a subject after treatment with a treatment regimen, each biomarker value being indicative of a value measured or derived for a respective hypoxia biomarker; (b) determining an indicator using a combination of the plurality of hypoxia biomarker values, the indicator being at least partially indicative of the presence or absence of a hypoxic condition, wherein: (i) at least two (e.g., 2, 3, 4, 5, 6, 7, 8, 9, 10, or more) hypoxic biomarkers have a mutual correlation in respect of the at least one condition that lies within a mutual correlation range, the mutual correlation range being between ±0.9; and (ii) the indicator has a performance value greater than or equal to a performance threshold representing the ability of the indicator to diagnose the presence or absence of the hypoxic condition, or to provide a prognosis for the at least one condition, the performance threshold being indicative of an explained variance of at least 0.3, and (c) determining that the treatment regimen is effective for changing the status of the subject to the desired state (e.g., normoxia) on the basis that the indicator indicates the presence of a normoxia or the presence of a hypoxia of a lower degree relative to the degree of hypoxia in the subject before treatment with the treatment regimen. Accordingly, this aspect of the present invention advantageously provides methods of monitoring the efficacy of a particular treatment regimen in a subject (for example, in the context of a clinical trial) already diagnosed with a hypoxic condition. These methods take advantage of measured or derived biomarker values that correlate with treatment efficacy to determine, for example, whether measured or derived biomarker values of a subject undergoing treatment partially or completely normalize during the course of or following therapy or otherwise shows changes associated with responsiveness to the therapy.

Accordingly, the invention provides methods of correlating a biomarker profile with an effective treatment regimen for a hypoxic condition. In some embodiments, these methods comprise: (1) determining a biomarker profile defining biomarker values that are measured or derived for at least one (e.g., 1, 2, 3, 4, 5, 6, 7, 8, 9, 10, or more) corresponding hypoxia biomarkers of a subject with hypoxia and for whom an effective treatment has been identified; and (2) correlating the biomarker profile so determined with an effective treatment regimen for a hypoxic condition (e.g., a G9a antagonist). In some embodiments, these methods comprise: (a) determining a biomarker profile defining a combination of at least two (e.g., 2, 3, 4, 5, 6, 7, 8, 9, 10, or more) biomarker values corresponding to values of at least two hypoxia biomarkers that can be measured or derived for a subject with hypoxia and for whom an effective treatment has been identified, wherein: (i) the at least two hypoxia biomarkers have a mutual correlation in respect of the condition that lies within a mutual correlation range, the mutual correlation range being between ±0.9; and (ii) the combination of at least two biomarker values has a performance value greater than or equal to a performance threshold representing the ability of the combination of at least two biomarker values to diagnose the presence or absence of a hypoxic condition, or to provide a prognosis for a disease or condition that is associated with hypoxia, the performance threshold being indicative of an explained variance of at least 0.3; and (b) correlating the biomarker profile so determined with an effective treatment regimen for a hypoxic condition (e.g., a G9a antagonist). In specific embodiments, an indicator or biomarker profile is correlated to a global probability or a particular outcome, using ROC curves.

The invention further provides methods for determining whether a treatment regimen is effective for treating a subject with a hypoxic condition (e.g., hypoxic cancer). In some embodiments, these methods comprise: (1) determining post-treatment biomarker values that are measured or derived for at least two (e.g., 2, 3, 4, 5, 6, 7, 8, 9, 10, or more) corresponding hypoxia biomarkers of a subject after treatment with a treatment regimen; (2) determining a post-treatment indicator using the post-treatment biomarker values, wherein the post-treatment indicator is at least partially indicative of the presence or absence of a hypoxic condition, wherein the post-treatment indicator indicates whether the treatment regimen is effective for treating the hypoxic condition in the subject on the basis that post-treatment indicator indicates the presence of a healthy condition or the presence of hypoxia of a lower degree relative to the degree of hypoxia in the subject before treatment with the treatment regimen. In other embodiments, these methods comprise: (a) determining a plurality of post-treatment biomarker values, each post-treatment hypoxia biomarker value being indicative of a value measured or derived for at least one (e.g., 1, 2, 3, 4, 5, 6, 7, 8, 9, 10, or more) hypoxia biomarker of a subject after treatment with the treatment regimen; (b) determining a post-treatment indicator using a combination of the plurality of post-treatment biomarker values, the post-treatment indicator being at least partially indicative of the presence or absence of a hypoxic condition, wherein: (i) at least two (e.g., 2, 3, 4, 5, 6, 7, 8, 9, 10, or more) hypoxia biomarkers have a mutual correlation in respect of the at least one condition that lies within a mutual correlation range, the mutual correlation range being between ±0.9; and (ii) the post-treatment indicator has a performance value greater than or equal to a performance threshold representing the ability of the post-treatment indicator to diagnose the presence or absence of a hypoxic condition, the performance threshold being indicative of an explained variance of at least 0.3, wherein the post-treatment indicator indicates whether the treatment regimen is effective for treating the hypoxic condition in the subject on the basis that post-treatment indicator indicates the presence of a healthy condition (i.e, normoxia) or the presence of a hypoxic condition of a lower degree relative to the degree of hypoxia in the subject before treatment with the treatment regimen.

The invention can also be practiced to evaluate whether a subject is responding (i.e., a positive response) or not responding (i.e., a negative response) to a treatment regimen. This aspect of the invention provides methods of correlating a biomarker profile with a positive or negative response to a treatment regimen. In some embodiments, these methods comprise: (1) determining a biomarker profile defining biomarker values that are measured or derived for at least two (e.g., 2, 3, 4, 5, 6, 7, 8, 9, 10, or more) corresponding hypoxia biomarkers of a subject following commencement of the treatment regimen; and (2) correlating the biomarker profile so determined with a positive or negative response to the treatment regimen. In other embodiments, these methods comprise: (a) determining a biomarker profile defining a combination of at least two (e.g., 2, 3, 4, 5, 6, 7, 8, 9, 10, or more) biomarker values corresponding to values of at least two hypoxia biomarkers that can be measured or derived for a subject following commencement of the treatment regimen, wherein: (i) the at least two hypoxia biomarkers have a mutual correlation in respect of hypoxia, which lies within a mutual correlation range, the mutual correlation range being between ±0.9; and (ii) the combination of at least two biomarker values has a performance value greater than or equal to a performance threshold representing the ability of the combination of at least two biomarker values to diagnose the presence or absence of a hypoxic condition, or to provide a prognosis for disease or condition that is associated with the hypoxic condition, the performance threshold being indicative of an explained variance of at least 0.3; and (b) correlating the biomarker profile so determined with a positive or negative response to the treatment regimen.

The invention also encompasses methods of determining a positive or negative response to a treatment regimen by a subject with a hypoxic condition. In some embodiments, these methods comprise: (1) correlating a reference biomarker profile with a positive or negative response to the treatment regimen, wherein the biomarker profile defines biomarker values that are measured or derived for at least two (e.g., 2, 3, 4, 5, 6, 7, 8, 9, 10, or more) corresponding hypoxic biomarkers of a control subject or control group; (2) determining a sample biomarker profile defining biomarker values that are measured or derived for the at least two (e.g., 2, 3, 4, 5, 6, 7, 8, 9, 10, or more) corresponding hypoxic biomarker of the subject following commencement of the treatment regimen, wherein the sample biomarker profile indicates whether the subject is responding positively or negatively to the treatment regimen, based on the correlation of the reference biomarker signature with the positive or negative response to the treatment regimen. In other embodiments, the methods comprise: (a) correlating a reference biomarker profile with a positive or negative response to the treatment regimen, wherein the biomarker profile defines a combination of at least two (e.g., 2, 3, 4, 5, 6, 7, 8, 9, 10, or more) biomarker values corresponding to values of at least two hypoxia biomarkers that are measured for or derived from a control subject or control group, wherein: (i) the at least two hypoxia biomarkers have a mutual correlation in respect of hypoxia, which lies within a mutual correlation range, the mutual correlation range being between ±0.9; and (ii) the combination of at least two biomarker values has a performance value greater than or equal to a performance threshold representing the ability of the combination of at least two biomarker values to diagnose the presence or absence of a hypoxic condition, or to provide a prognosis for a disease or condition that is associated with hypoxia, the performance threshold being indicative of an explained variance of at least 0.3; (b) determining a sample biomarker profile defining a combination of at least two (e.g., 2, 3, 4, 5, 6, 7, 8, 9, 10, or more) biomarker values corresponding to values of the at least two hypoxia biomarkers that are measured or derived from the subject following commencement of the treatment regimen, wherein the sample biomarker profile indicates whether the subject is responding positively or negatively to the treatment regimen, based on the correlation of the reference biomarker profile with the positive or negative response to the treatment regimen.

In related embodiments, the present invention further contemplates methods of determining a positive or negative response to a treatment regimen by a biological subject. In some embodiments, these methods comprise: (1) determining a sample biomarker profile defining biomarker values that are measured or derived for at least two (e.g., 2, 3, 4, 5, 6, 7, 8, 9, 10, or more) corresponding hypoxia biomarker of a subject following commencement of the treatment regimen, wherein the sample biomarker profile is correlated with a positive or negative response to the treatment regimen; and (2) determining whether the subject is responding positively or negatively to the treatment regimen based on the sample biomarker profile. In other embodiments, these methods comprise: (a) determining a sample biomarker profile defining a combination of at least two (e.g., 2, 3, 4, 5, 6, 7, 8, 9, 10, or more) biomarker values corresponding to values of at least two hypoxia biomarkers that are measured for or derived from a subject following commencement of the treatment regimen, wherein: (i) the at least two hypoxia biomarkers have a mutual correlation in respect of hypoxia, which lies within a mutual correlation range, the mutual correlation range being between ±0.9; and (ii) the combination of at least two biomarker values has a performance value greater than or equal to a performance threshold representing the ability of the combination of at least two biomarker values to diagnose the presence or absence of a hypoxic condition, or to provide a prognosis for a disease or condition that is associated with hypoxia, the performance threshold being indicative of an explained variance of at least 0.3, wherein the sample biomarker profile is correlated with a positive or negative response to the treatment regimen; and (b) determining whether the subject is responding positively or negatively to the treatment regimen based on the sample biomarker profile.

The above methods can be practiced to identify responders or non-responders relatively early in the treatment process, i.e., before clinical manifestations of efficacy. In this way, the treatment regimen can optionally be discontinued, a different treatment protocol can be implemented and/or supplemental therapy can be administered. Thus, in some embodiments, a sample hypoxia biomarker profile is obtained within about 2 hours, 4 hours, 6 hours, 12 hours, 1 day, 2 days, 3 days, 4 days, 5 days, 1 week, 2 weeks, 3 weeks, 4 weeks, 6 weeks, 8 weeks, 10 weeks, 12 weeks, 4 months, six months or longer of commencing therapy.

The present invention also contemplates methods in which the indicator-determining method of the invention is implemented using one or more processing devices. In some embodiments, these methods comprise: (1) determining a pair of biomarker values, the pair of biomarker values consisting of a first biomarker value indicative of a concentration of polynucleotide expression products of a group 1 hypoxia biomarker gene (e.g., ARNTL, CD1C, HHEX, KLRG1, MMP16, FGFR2, GATA2, CEACAM7, OGN, and AGTR1) and a group 2 hypoxia biomarker gene (e.g., G9a); (2) determining an indicator indicative of a ratio of the concentrations of the polynucleotide expression products using both biomarker values; (3) retrieving previously determined indicator references from a database, the indicator references being determined based on indicators determined from a reference population, one of the groups consisting of individuals diagnosed with a hypoxic condition; (4) comparing the indicator to the indicator references; (5) using the results of the comparison to determine a probability indicative of the subject having or not having a hypoxic condition; and (6) generating a representation of the probability, the representation being displayed to a user to allow the user to assess the likelihood of a biological subject having a hypoxic condition.

Similarly apparatus can be provided for determining the likelihood of a subject having a hypoxic condition, the apparatus including: (A) a sampling device that obtains a sample taken from a subject, the sample including polynucleotide expression products; (B) a measuring device that quantifies polynucleotide expression products within the sample to determine three biomarker values, at least one biomarker value consisting of: (a) a first pair of biomarker values indicative of a concentration of polynucleotide expression products of a group 1 hypoxia biomarker gene (e.g., ARNTL, CD1C, HHEX, KLRG1, MMP16, FGFR2, GATA2, CEACAM7, OGN, and AGTR1) and a group 2 hypoxia biomarker gene (e.g., G9a); (C) at least one processing device that: (i) receives an indication of the pair of biomarker values from the measuring device; (ii) determines an indicator using a ratio of the concentration of the first, second and third polynucleotide expression products using the biomarker values; (iii) compares the indicator to at least one indicator reference; (iv) determines a likelihood of the subject having or not having a hypoxic condition using the results of the comparison; and (v) generates a representation of the indicator and the likelihood for display to a user.

The present invention also encompasses methods for differentiating between hypoxia and normoxia in a subject. These methods suitably comprise: (a) obtaining a sample taken from a subject showing a clinical sign of a hypoxic condition (e.g., a hypoxic condition), the sample including polynucleotide expression products; (b) in a measuring device: (i) amplifying at least some polynucleotide expression products in the sample; (ii) determining an amplification amount representing a degree of amplification required to obtain a defined level of polynucleotide expression products including: amplification amounts for a first pair of polynucleotide expression products of a group 1 hypoxia biomarker gene (e.g., ARNTL, CD1C, HHEX, KLRG1, MMP16, FGFR2, GATA2, CEACAM7, OGN, and AGTR1) and a group 2 hypoxia biomarker gene (e.g., G9a); (c) in a processing system: (i) retrieving the amplification amounts; (ii) determining an indicator by: determining a first derived biomarker value indicative of a ratio of concentrations of the first pair of polynucleotide expression products by determining a difference between the amplification amounts for the first pair; (d) determining the indicator using the derived biomarker value; (e) retrieving previously determined indicator references from a database, wherein the indicator references are distributions of indicators determined for first and second groups of a reference population, the first and second groups consisting of individuals diagnosed with a hypoxic condition and the other with a normoxic condition, respectively; (f) comparing the indicator to the first and second indicator references; (g) using the results of the comparison to determine a probability of the subject being classified within the first or second group; (h) generating a representation at least partially indicative of the indicator and the probability; and (i) providing the representation to a user to allow the user to assess the likelihood of a subject having or not having a hypoxic condition or a normoxic condition.

Additionally, methods can be provided for determining an indicator used in assessing a likelihood of a subject having a presence or absence of a hypoxic condition, or in providing a prognosis for a disease or condition that is associated with hypoxia. These methods suitably include: (1) determining a plurality of biomarker values, each biomarker value being indicative of a value measured or derived for at least one corresponding hypoxia biomarker of the subject and being at least partially indicative of a concentration of the hypoxia biomarker in a sample taken from the subject; (2) determining the indicator using a combination of the plurality of biomarker values, wherein: at least two biomarkers have a mutual correlation in respect of a hypoxic condition that lies within a mutual correlation range, the mutual correlation range being between ±0.9; and the indicator has a performance value greater than or equal to a performance threshold representing the ability of the indicator to diagnose the presence or absence of a hypoxic condition, or to provide a prognosis for the disease or condition that is associated with hypoxia, the performance threshold being indicative of an explained variance of at least 0.3.

2.8 Ancillary Treatments

In some embodiments, the treatment regimens described above or elsewhere herein comprise a combination therapy comprising administering a G9a antagonist together with an ancillary treatment.

It is well known that chemotherapy and radiation therapy target rapidly dividing cells and/or disrupt the cell cycle or cell division. These treatments are offered as part of the treating several forms of cancer and autoimmune disease, aiming either at slowing their progression or reversing the symptoms of disease by means of a curative treatment. In some embodiments, therefore, upon determining a likelihood that a subject has a hypoxic condition (for example, a cancer or tumor) a combination therapy can employ G9a inhibitor that is administered together with a chemotherapeutic agent, which is suitably selected from cytostatic agents and cytotoxic agents. Non-limiting examples of cytostatic agents are selected from: (1) microtubule-stabilizing agents such as but not limited to taxanes, paclitaxel, docetaxel, epothilones and laulimalides; (2) kinase inhibitors, illustrative examples of which include Iressa®, Gleevec, Tarceva™, (Erlotinib HCl), BAY-43-9006, inhibitors of the split kinase domain receptor tyrosine kinase subgroup (e.g., PTK787/ZK 222584 and SU11248); (3) receptor kinase targeted antibodies, which include, but are not limited to, Trastuzumab (Herceptin®), Cetuximab (Erbitux®), Bevacizumab (Avastin™), Rituximab (Ritusan®), Pertuzumab (Omnitarg™); (4) mTOR pathway inhibitors, illustrative examples of which include rapamycin and CCI-778; (5) Apo2L/Trail, anti-angiogenic agents such as but not limited to endostatin, combrestatin, angiostatin, thrombospondin and vascular endothelial growth inhibitor (VEGI); (6) antineoplastic immunotherapy vaccines, representative examples of which include activated T-cells, non-specific immune boosting agents (i.e., interferons, interleukins); (7) antibiotic cytotoxic agents such as but not limited to doxorubicin, bleomycin, dactinomycin, daunorubicin, epirubicin, mitomycin and mitozantrone; (8) alkylating agents, illustrative examples of which include Melphalan, Carmustine, Lomustine, Cyclophosphamide, Ifosfamide, Chlorambucil, Fotemustine, Busulfan, Temozolomide and Thiotepa; (9) hormonal antineoplastic agents, non-limiting examples of which include Nilutamide, Cyproterone acetate, Anastrozole, Exemestane, Tamoxifen, Raloxifene, Bicalutamide, Aminoglutethimide, Leuprorelin acetate, Toremifene citrate, Letrozole, Flutamide, Megestrol acetate and Goserelin acetate; (10) gonadal hormones such as but not limited to Cyproterone acetate and Medoxyprogesterone acetate; (11) antimetabolites, illustrative examples of which include Cytarabine, Fluorouracil, Gemcitabine, Topotecan, Hydroxyurea, Thioguanine, Methotrexate, Colaspase, Raltitrexed and Capicitabine; (12) anabolic agents, such as but not limited to, Nandrolone; (13) adrenal steroid hormones, illustrative examples of which include Methylprednisolone acetate, Dexamethasone, Hydrocortisone, Prednisolone and Prednisone; (14) neoplastic agents such as but not limited to Irinotecan, Carboplatin, Cisplatin, Oxaliplatin, Etoposide and Dacarbazine; and (15) topoisomerase inhibitors, illustrative examples of which include topotecan and irinotecan.

Illustrative cytotoxic agents can be selected from sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)] tetrachloride, diarizidinylspermine, arsenic trioxide, 1-(11-dodecylamino-10-hydroxyundecyI)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycarminomycin, annamycin, galarubicin, elinafide, MEN10755, 4-demethoxy-3-deamino-3-aziridinyl-4-methylsulphonyl-daunombicin (see International Publication WO 00/50032), methoxtrexate, gemcitabine, and mixture thereof.

Radiotherapies include radiation and waves that induce DNA damage for example, γ-irradiation, X-rays, UV irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half life of the isotope, the strength and type of radiation emitted, and the uptake by the neoplastic cells.

Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

Chemotherapeutic agents may be selected from any one or more of the following categories:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyridines like 5-fluorouracil and tegafur, raltitrexed, methotrexate, cytosine arabinoside and hydroxyurea; anti-tumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and docetaxel; and topoisomerase inhibitors (for example epipodophyllotoxins like etoposide and teniposide, amsacrine, topotecan and camptothecin);

(ii) cytostatic agents such as antiestrogens (for example tamoxifen, toremifene, raloxifene, droloxifene and idoxifene), oestrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5a-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazoli-n-4-amine (CI 1033)), for example inhibitors of the platelet-derived growth factor family and for example inhibitors of the hepatocyte growth factor family;

(v) anti-angiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example those which are directed to the targets listed above, such as ISIS 2503, an anti-ras antisense; and

(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Immunotherapy approaches, include for example ex vivo and in vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

Examples of other cancer therapies include phototherapy, cryotherapy, toxin therapy or pro-apoptosis therapy. One of skill in the art would know that this list is not exhaustive of the types of treatment modalities available for cancer and other hyperplastic lesions.

In exemplary embodiments of this type, the group 1 hypoxia biomarker is a nucleic acid expression product, and the group 2 hypoxia biomarker is a polypeptide expression product.

In order that the invention may be readily understood and put into practical effect, particular preferred embodiments will now be described by way of the following non-limiting examples.

### EXAMPLES

### Example 1

### Hypoxia Stabilizes G9a Methyltransferase

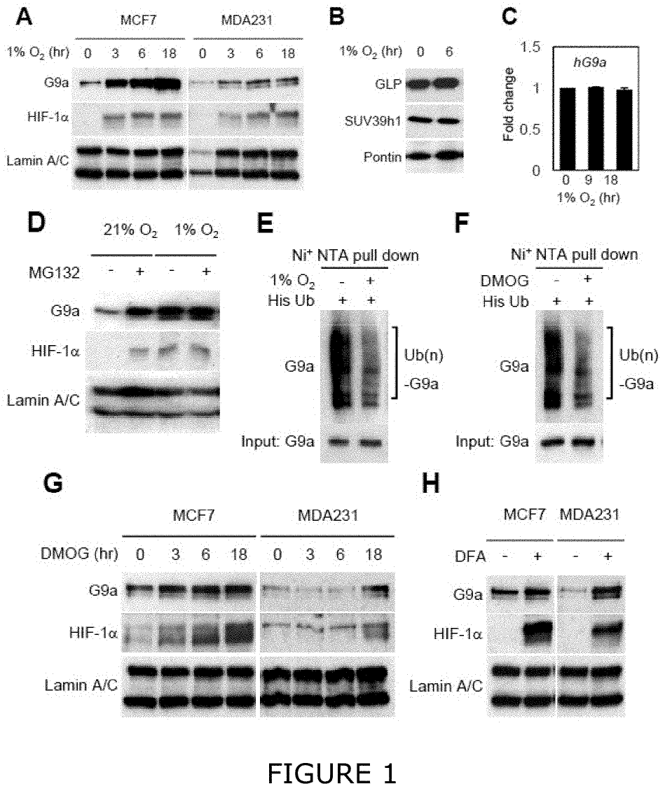

In order to determine the mechanism by which hypoxia alters the level of G9a protein, MCF7 and MDA-MB-231 (MDA231) breast cancer cells were exposed to hypoxic conditions. Strikingly, a significant increase in G9a protein level was observed. This increase was detectable as early as three hours (FIG. 1A) while there was no significant change in two other methyltransferases that target H3K9 GLP and SUV39h1 (see, FIG. 1B)). To investigate whether this increase in G9a protein level was due to an increase in transcription of G9a, quantitative PCR (qPCR) was performed. It was found that G9a transcription was not altered, suggesting that it is post-transcriptionally regulated (see, FIG. 1C). Indeed, contacting cells with MG132 (a proteasomal inhibitor) resulted in an increase in G9a protein level in normoxia, but not in hypoxia (FIG. 1D), indicating that hypoxia-mediated regulation of G9a protein stability may involve perturbation of the proteasomal degradation pathway.

As protein ubiquitination is linked to proteasomal degradation we assessed the extent of G9a ubiquitination in normoxia and hypoxia. Ubiquitination assays revealed that polyubiquitination of G9a was significantly reduced in hypoxia (see, FIG. 1E). As G9a showed very similar stabilization dynamics to that of HIF-1a protein in hypoxia, the present inventors examined the effect of inhibiting prolyl hydroxylase domain (PHD) enzymes on G9a protein stability. The level of G9a polyubiquitination was markedly reduced with treatment of the prolyl hydroxylase inhibitor dimethyloxaloylglycine (DMOG) (see, FIG. 1F) consistent with the increase in G9a protein levels in both MCF7 and MDA231 breast cancer cells treated with either DMOG or deferoxamine (DFA) (see, FIGS. 1G and H).

Together, these data strongly suggest that hypoxic condition leads to G9a protein stabilization by inhibiting the activities of PHD enzymes and that this could facilitate changes in gene expression that are independent of HIFs.

### Materials and Methods

Cell Culture and Drug Treatments

Breast cancer cell lines were obtained from ATCC and cultured as per ATCC instructions. AT3 mammary adenocarcinoma, was maintained as previously described (in Ngiow, 2012). All cell lines were regularly tested for mycoplasma and authenticated using short tandem repeat profiling. UNC0642 was purchased from Sigma-Aldrich and MG132 (M-1157) from A.G. Scientific.

Ubiquitination Assay

The ubiquitination assay was performed as described previously (Lee, Mol. Cell, 2012). Briefly, cells were transfected with expression constructs including His-tagged ubiquitin and treated with 5 μg/ml of MG132 for 4 hrs, lysed in buffer A (6 M guanidinium-HCl, 100 mM sodium phosphate, 10 mM Tris-HCl (pH 8.0), 5 mM imidazole, and 10 mM β-mercaptoethanol), and incubated with Ni²⁺-NTA beads (QIAGEN) for 4 hrs at room temperature. The beads were then washed sequentially with buffer A, buffer B (8 M urea, 100 mM sodium phosphate buffer, 10 mM Tris-HCl (pH 8.0), and 10 mM β-mercaptoethanol), and buffer C (8 M urea, 100 mM sodium phosphate buffer, 10 mM Tris-HCl (pH 6.3), and 10 mM β-mercaptoethanol). Bound proteins were eluted with buffer D (200 mM imidazole, 150 mM Tris-HCl (pH 6.7), 30% glycerol, 0.72 M β-mercaptoethanol, and 5% SDS), and subject to immunoblot analysis.

### Example 2

### Prolyl Hydroxylation-Mediated G9a Degradation

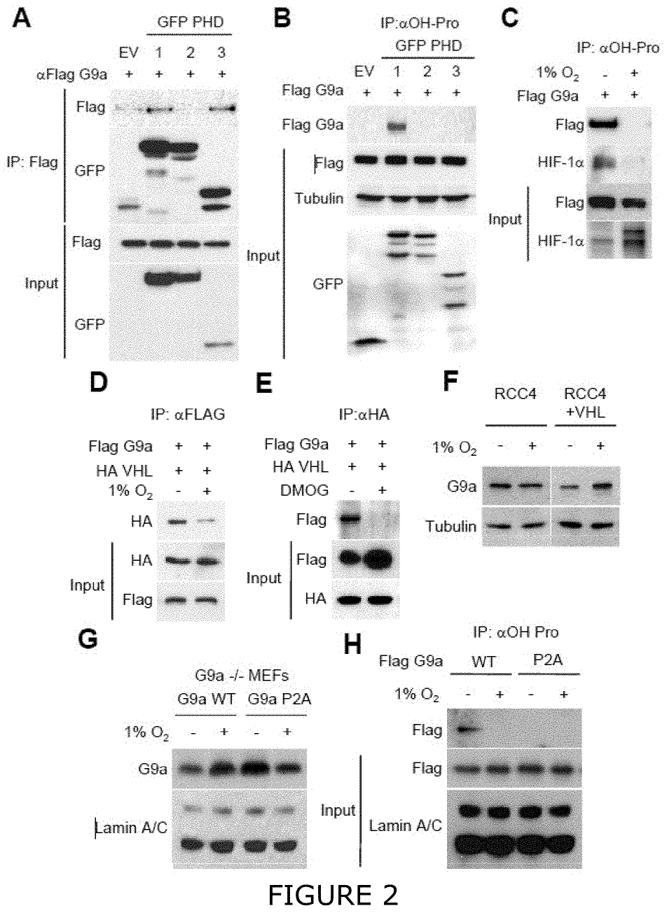

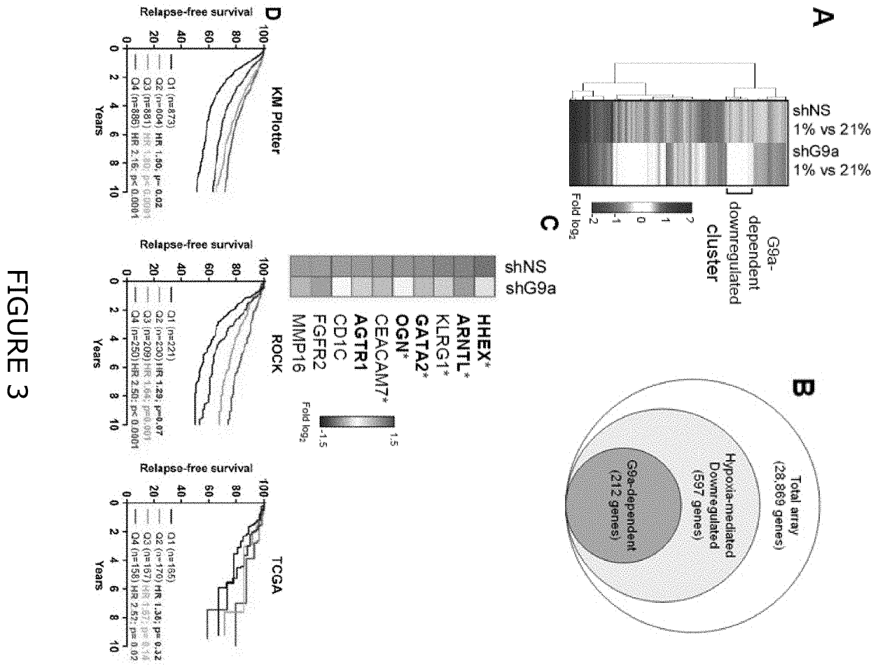

Since inhibition of PHD enzyme activity leads to a change in G9a protein stability, we examined the interaction between G9a and known PHD enzymes by performing co-immunoprecipitation assays. From these experiments, it was clear that PHD1 and PHD3 were capable of physically interacting with G9a (see, FIG. 2A). However, hydroxyproline pull-down assays using anti-hydroxy prolyl antibody revealed that only PHD1 was able to hydroxylate G9a (see, FIG. 2B). Moreover, although G9a hydroxylation was readily detected in normoxic condition, it was almost completely undetectable in hypoxic condition (see, FIG. 2C). Because pVHL is known to be involved in degrading hydroxylated HIFa proteins (see, Ivan et al., 2001; and Jaakkola et al., 2001), a co-immunoprecipitation assay between G9a and pVHL was performed to determine whether G9a degradation was also regulated in a pVHL-dependent manner. In this regard, G9a showed an interaction with pVHL in normoxia but this interaction was significantly reduced in hypoxia (see, FIG. 2D). Moreover, the interaction between G9a and pVHL was significantly inhibited by DMOG treatment suggesting that G9a hydroxylation appears to be an essential modification required for G9a recognition by pVHL (see, FIG. 2E). The involvement of pVHL in regulating the G9a protein level in hypoxia was further confirmed in the RCC4 cell line in which pVHL is defective. Hypoxia-dependent stabilization of G9a was absent in RCC4 cells. However, expression of wild-type pVHL restored G9a sensitivity to hypoxia (see, FIG. 2F).

Based on the known consensus hydroxylation motif “LXXLXP”, we identified two potential proline hydroxylation sites on G9a at amino acid residues P606 and P1206. The two proline residues were mutated to alanine by site-directed mutagenesis to generate a G9a P2A mutant. The G9a P2A mutant failed to show hypoxia-dependent accumulation when expressed in G9a-deficient mouse embryonic fibroblasts (MEF), but appeared to show a higher level of protein in normoxia compared to that of wild-type G9a (see, FIG. 2G).

The present inventors then performed hydroxylation assays and found that while a considerable amount of proline hydroxylation was detected for wild-type G9a, no detectable level of proline hydroxylation was observed for the G9a P2A mutant (see, FIG. 2H).

These data clearly show that PHD1 and pVHL have important roles in regulating G9a protein stability.

### Materials and Methods

Antibodies

The following commercially available antibodies were used: anti-GFP (sc-8334) was purchased from Santa Cruz Biotechnology. Anti-HA (MMS-101P-500) was from Jomar Biosciences. Anti-H3 (ab1791), anti-H3K9me1 (ab9045), anti-H3K9me2 (ab1220), anti-H3K9me3 (ab8898), anti-RNA Polymerase II (ab817), anti-tubulin (ab6046), anti-pontin (ab51500), anti-LC3 (ab38394) and anti-hydroxyl proline (ab37067) were from Abcam. Anti-G9a (07-551), anti-GLP (B0422), anti-Lamin A/C (05-714) and anti-SUV39h1 (S8316) were from EMD Millipore. Anti-FLAG (F3165) was from Sigma Aldrich. Anti-HIF-1a (NB100-479), anti-PHD1(NB100-310), anti-PHD2 (NBP1-30328), and anti-PHD3 (NB100-303) were from Novus Biologicals.

shRNA Knock Down

shRNA-mediated knockdown cells were generated as previously described (Lee, Mol. Cell, 2010). To generate knockdown cells, retroviral shRNA constructs (shNS and shG9a) with viral packaging plasmids (pMD2.G and pMD-MLV) were transfected into HEK293T cells. Viral supernatant was collected after three days for using for target cell infection.

### Example 3

### Identification of G9a Target Genes and its Use as a Prognostic Indicator

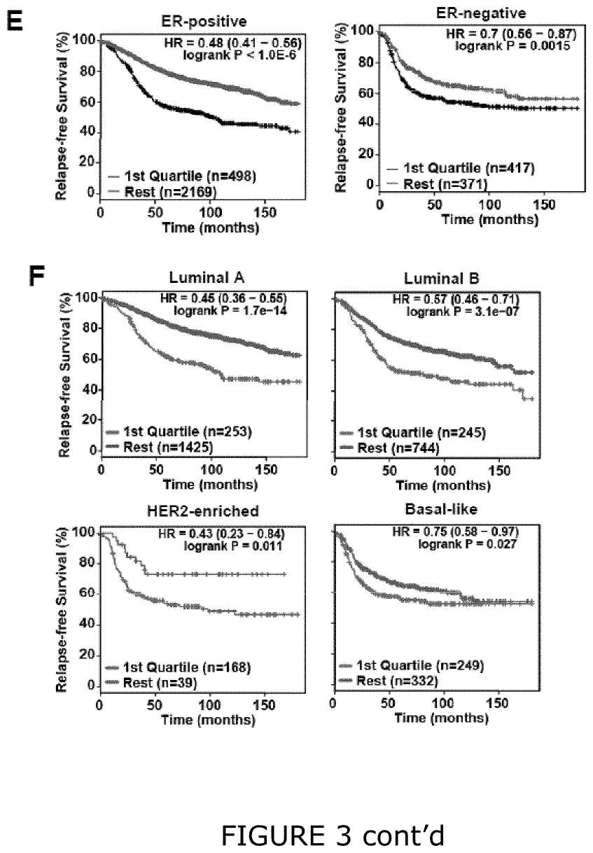

In order to determine the functional consequence of G9a accumulation in hypoxic conditions, the present inventors performed a microarray analysis from RNA isolated from MCF7 cells expressing either non-silencing control shRNA (shNS) or G9a shRNA (shG9a) exposed to normoxic and hypoxic conditions. The global analysis investigated the general impact of hypoxia and G9a knockdown on gene expression. A Gaussian curve fitting was used to determine the applicable cut-offs in selecting genes that showed significant changes in response to hypoxia (methods as taught by Hwang et al., 2005). Hypoxia-responsive genes could be largely categorized as being upregulated genes or downregulated genes (see, FIG. 3A). Given the strong association between the G9a methyltransferase activity toward H3K9 methylation in gene silencing (see, Chen et al., 2010; and Yamamizu et al., 2012), G9a was investigated as a potential mediator of hypoxia-dependent gene repression (see, FIG. 3B). Among those genes downregulated by hypoxia, 36% of genes (i.e., 212 genes) appeared to be G9a-dependent as these genes were no longer downregulated by hypoxia in the G9a knock-down model. The remaining 64% of genes (i.e., 385 genes) were downregulated by hypoxia in a G9a-independent manner.

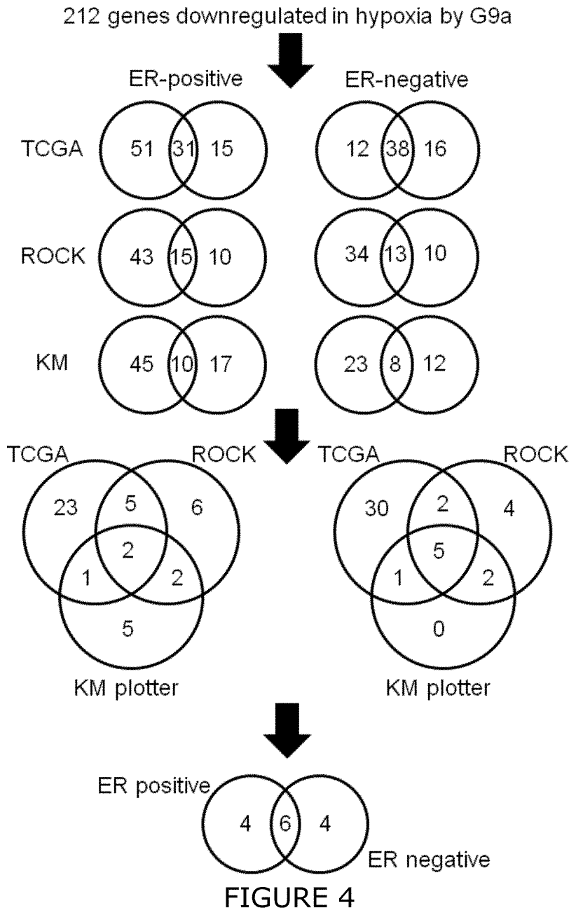

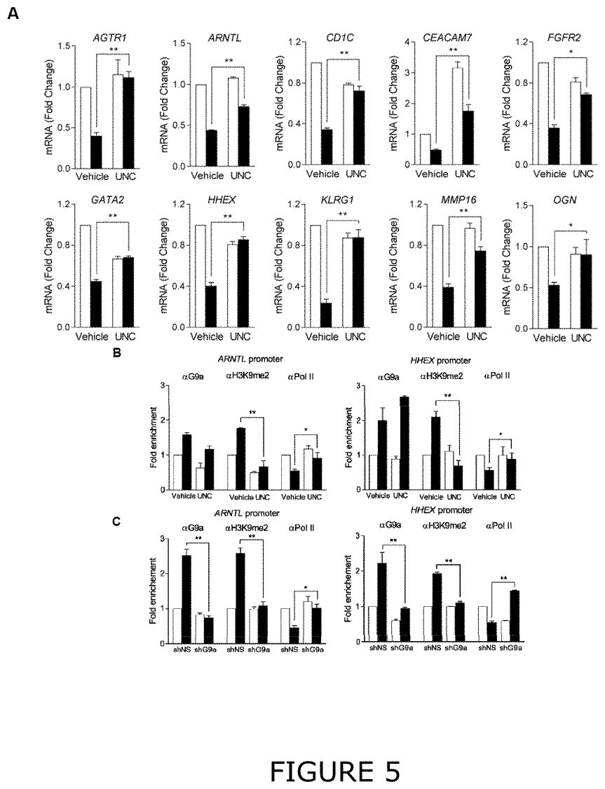

Given that tumor hypoxia is correlated with treatment resistance (Vaupel, 2004) and metastatic transformation of cancer cells (Joyce and Pollard, 2009), the present inventors tested whether genes repressed by G9a (but not hypoxia per se) are correlated with patient outcome. As breast cancer is heterogeneous and molecular subtypes have been well characterized, patient survival analysis was divided into estrogen receptor-positive (ER-positive) and estrogen receptor-negative (ER-negative) groups. The analysis was performed on these two groups in three large gene expression datasets for breast cancer; KM plotter, ROCK and TCGA (see, FIG. 4). The previously published hypoxic markers (13 genes, see, for example, Hu et al., 2009) as a marker for hypoxia in the tumors. With the same rationale of the in vitro studies, the present inventors identified those genes whose expression were inversely correlated to G9a expression and those whose expression were inversely correlated to hypoxia (determined by the hypoxic markers). These genes were then analyzed for commonality between the three datasets as shown in the Venn diagrams in FIG. 3D.

Ten genes were found that were common between at least two of the three datasets analyzed for each breast cancer subtypes (e.g., 10 genes for ER-positive; and 10 genes for ER-negative). Out of the 20 genes, there were 14 distinct genes with 6 genes overlapping. Out of these 14 genes, only 10 genes associated with relapse-free survival (see, FIG. 3C and FIG. 4). In order to determine whether these genes were prognostic, an association analysis was performed to find those genes that associated with poor relapse-free survival in the three datasets analyzed. The 10 genes identified in FIG. 3C (i.e., ARNTL, CD1C, HHEX, KLRG1, MMP16, FGFR2, GATA2, CEACAM7, OGN, and AGTR1) were analyzed for their association with relapse-free survival as a gene signature. The average expression of the 10 genes was used as a G9a-suppressed gene signature for each patient. Breast cancer cases in each of the three datasets (KM plotter, ROCK and TCGA) were allocated to one of four quartiles based on the G9a-suppressed gene signature, and relapse-free survival was compared (FIG. 3D-F). KM plotter breast cancer gene expression database contained the largest patient number analyzed (n=3524). Patients with the lowest expression of the G9a-suppressed signature (1st quartile; Q1 in black) were associated with a poorer relapse-free survival with only about 50% of patients surviving after 10 years, while the patients with the highest G9a-suppressed gene signature (quartile 4; Q4 in red) associated with significantly better survival with close to 80% of patients surviving (FIG. 3D). The ROCK and TCGA gene expression datasets also showed similar association between the G9a-suppressed gene signature and relapse-free survival, further strengthening the prognostic power of the G9a-suppressed gene signature. In the ER-positive patient group, patient group with the lowest expression of the G9a-suppressed gene signature (1st quartile in black) was associated with significantly poorer survival compared to the rest of the group (rest in red). In the ER-negative patient group the patient stratification using the G9a-suppressed gene signature was not as efficient as the ER-positive group. Further, breast cancer patients were divided into four histopathological subtypes (luminal A, luminal B, HER2-enriched, and basal-like). Survival of the patients with the lowest G9a-suppressed gene signature was compared to the rest of the patients within each group (FIG. 3F). The G9a-suppressed gene signature was able to identify a distinct patient group associated with a poor survival (1st quartile) in all subgroups except for the basal-like group. Notably, six genes (i.e., ARNTL, CEACAM7, GATA2, HHEX, KLRG1 and OGN) of the gene signature have been reported to possess tumor suppressor activity (Li et al., 2014; Hu et al., 2016; Li et al., 2014; Noy et al., 2010; Kershaw et al., 2014; Scholzel et al., 2000).

### Materials and Methods

Microarray Analysis

Briefly, MCF7 cells transfected with non-silencing control shRNA or shRNA targeting G9a were exposed to normoxia or hypoxia for 9 h, and total RNA was isolated with the RNAeasy Mini Kit (Qiagen); 500 ng of total RNA was used for microarray analysis. The Microarray analysis was performed using the Affymetrix Human Gene 1.0 ST Array by the Seoul National University Genome Research Facility.

### Example 4

### Functional Role of G9a Accumulation

Upon verifying the prognostic value of the G9a-suppressed gene signature using patient survival data, the present inventors tested the ability of a G9a inhibitor to reverse G9a-mediated suppression. Expression of the 10 G9a-suppressed genes was analyzed by real-time qPCR. The expression of all 10 genes was significantly downregulated by hypoxia in control cells, while this repression was abolished by treating cells with UNC0642, a small molecule inhibitor of G9a methyltransferase activity (see, FIG. 4A). This shows that their expression is controlled via G9a-mediated actions. To understand the molecular mechanism by which hypoxia-mediated accumulation of G9a results in the repression of specific genes, chromatin immunoprecipitation (ChIP) was performed on promoters of the 10 G9a-repressed genes (see, FIG. 4B and Figure S4). Hypoxia significantly increased H3K9me2 at the promoters of these genes and correlated with transcriptional silencing (FIG. 4B). Hypoxia-mediated increase in H3K9me2 was almost completely abrogated or significantly reduced in promoters of G9a-repressed genes with G9a knockdown or with UNC0642 treatment (see, FIGS. 4B and 4C). Together, these results support the mechanism by which G9a represses transcription of specific genes via increasing H3K9me2, and that this increase in H3K9me2 can be abrogated by the use of small molecule inhibitor of G9a.

Effects of G9a inhibition in breast cancer cell lines using a small molecule inhibitor.

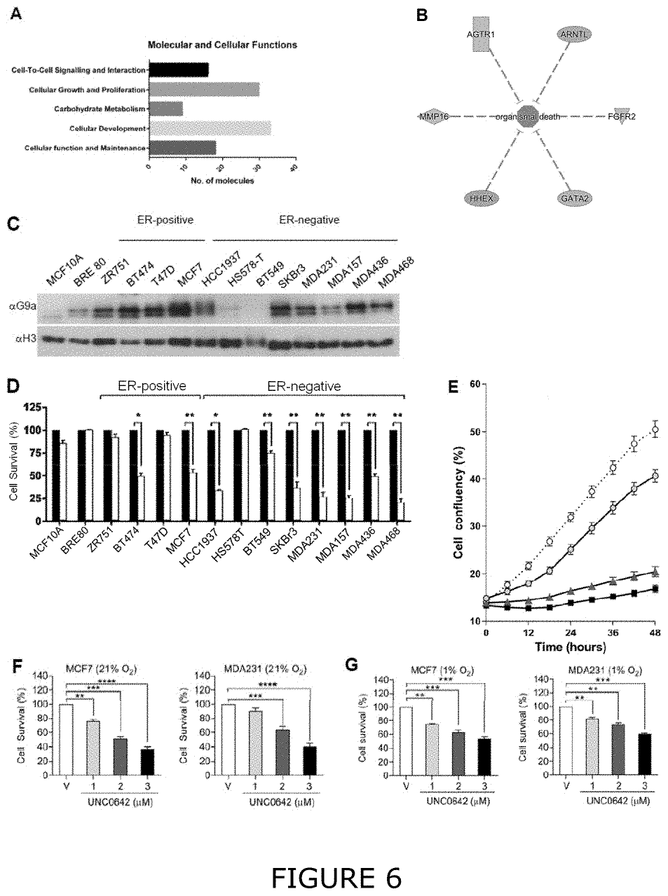

In order to determine the molecular function of G9a, the present inventors utilized the gene expression data acquired in FIG. 3 and performed a functional annotation network analysis through the use of Ingenuity Pathway Analysis (IPA®, QIAGEN, Redwood City). Several molecular and cellular pathways related to cellular growth and development were identified as affected (see, FIG. 6A). It also predicted that G9a-suppressed gene signature was associated with increased survival (see, FIG. 6B). Western immunoblotting analysis was performed to determine G9a protein levels in 14 different breast epithelial cells including normal-like (namely, MCF10A and Bre80 cells), four ER-positive (namely, ZR751, BT474, T47D and MCF7 cells) and eight ER-negative subtypes (namely, HCC1937, HS578-T, BT549, SKBr3, MDA231, MDA157, MDA436 and MDA468 cells). Most breast cancer cell lines expressed higher level of G9a protein in normoxia compared to that observed in normal-like breast epithelial cells (see, FIG. 6A). Upon evaluating the therapeutic potential of inhibiting G9a in these breast cancer cell lines using UNC0642 revealed that cell survival was unaffected in normal-like Bre80 and MCF10A breast epithelial cells, while survival of cancer cell lines was significantly attenuated by UNC0642 treatment (see, FIG. 6D). MCF7 and MDA231 cells were selected to further characterize the effect of UNC0642. Proliferation was evaluated by performing real time cell imaging using the IncuCyte Zoom where cells were grown for 48 hours in the presence or in absence of various concentrations of UNC0642 (see, FIGS. 6E and 6A). Inhibiting G9a resulted in a dose-dependent reduction in proliferation compared to vehicle control. The confluency of UNC0642 treated cells (2-3 μM) at the end of the experiment was 2 to 2.5-fold less compared to vehicle control (see, FIG. 6E). The inhibitory effect of UNC0642 on cellular proliferation and survival in both normoxic and hypoxic conditions was confirmed by performing SRB assay in which less than 20% of cells were present after 48 hour treatment in MCF7 (see, FIG. 6F) and MDA231 (see, FIG. 6G). A significant reduction in global H3K9me2 was observed (see, FIG. 6H) suggesting that the dose used was effectively inhibiting G9a methyltransferase activity. Since a considerable number of genes identified from the gene expression analyzes were those involved in cell-to-cell signaling and interaction (see, FIG. 6A), the present inventors examined whether pharmacologic inhibition of G9a impacts breast cancer cell motility by performing scratch wound assay, under normoxic and hypoxic conditions.

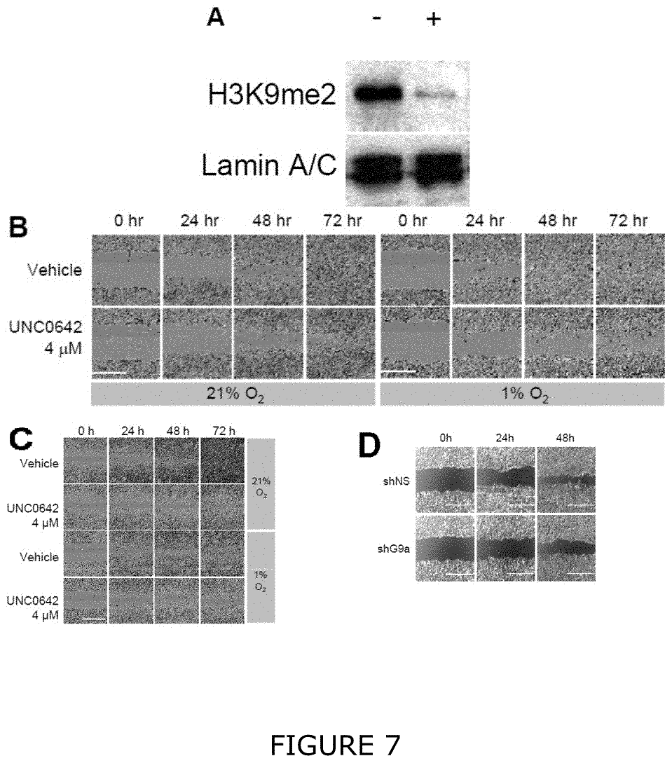

A scratch wound assay revealed that UNC0642 was able to inhibit cellular migration as demonstrated by the greater denuded area observed in the treated cells compared with vehicle control (FIGS. 7A and B). The inhibitory effect of UNC0642 on cellular migration was also evident in hypoxic condition in which cells were not able to close the wound even after 72 hours.

A further scratch wound assay comparing MCF7 (ER-positive) cells expressing shNS and shG9a in hypoxic condition demonstrated that G9a knockdown leads to a reduction in the cells ability to migrate (see, FIGS. 7C and D). Together, these results evidence the oncogenic function of G9a in enhancing cellular proliferation and migration.

### Materials and Methods

Quantitative Real-Time RT-PCR and ChIP Assays

Quantitative RT-PCR and ChIP assays were conducted as previously described.

Briefly, total RNA was isolated using Trizol (Invitrogen) and reverse transcription was performed from 2.5 μg of total RNA using the Superscript III cDNA synthesis kit (Invitrogen). The abundance of mRNA was detected by an ABI VIIA7 system with SYBR Green Master Mix (Life Technologies). Primer pairs were designed to amplify 90-150 bp mRNA specific fragments and were confirmed as unique products by melting curve analysis. The quantity of mRNA was calculated using ΔΔCt method and normalized by using primers to detect HPRT. All reactions were performed in triplicates.

See Table 7 for primer sequences used.

TABLE 7
ChIP Primers (5′-3′)

| | | |
|---|---|---|
| AGTR1 | FWD | TCATCCTTGCTGCCGTCAAT |
| | REV | CGTTGCTGCTTCTTGGGTTC |
| ARNTL | FWD | GACCTGAGGGGAAAGGGAGA |
| | REV | CTGCTACTTTCCTGCCACCA |
| CD1C | FWD | TGGAGAGTGGAGGCAAAGTT |
| | REV | TCCCTCTGGATTTTGCATGTCA |
| CEACAM7 | FWD | CTCTGTCACCTTCCTGCTGG |
| | REV | ACCTATGCTGTGTTCTGGCC |
| FGFR2 | FWD | AAACAACGTAACGCAGTCGC |
| | REV | AGCGACAGCCTCCGAATAAG |
| GATA2 | FWD | CACTTCCTTGCTTCCCCCAT |
| | REV | GTTTAGCTAAGTGCAGGCGC |
| HHEX | FWD | GTCCGAGGCCTCCAAATGAA |
| | REV | GCGCTCCCTGGATTAACAGT |
| KLRG1 | FWD | CACCACCACACCCAGCTAAT |
| | REV | CGCCTGTAATCCCAGCACTT |
| MMP16 | FWD | CGTTTTTAGATGCGAGGCGG |
| | REV | GACAGTATCTCCCATCCCGC |
| OGN | FWD | GCAGACTGAGTGCAGCAGTT |
| | REV | AAAATTTCAGGGCCCAGCAG |

qRT-PCR Primers (5′-3′)

| | | |
|---|---|---|
| G9a | FWD | CATTTCCGCATGAGTGATGATGT |
| | REV | GGCAGAACCTAACTCCTCCGA |
| AGTR1 | FWD | GGCTATTGTTCACCCAATGAAGT |
| | REV | TGGGACTCATAATGGAAAGCAC |
| ARNTL | FWD | CATTAAGAGGTGCCACCAATCC |
| | REV | TCATTCTGGCTGTAGTTGAGGA |
| CD1C | FWD | GCATCCCAGGAACACGTCTC |
| | REV | GCCATGAGTCTGCAACTCGT |
| CEACAM7 | FWD | TCAGCCTGTCCATACAGAGTG |
| | REV | TTGAACGGCACGACATCAATA |
| FGFR2 | FWD | GGAAAGTGTGGTCCCATCTGA |
| | REV | TCCAGGTGGTACGTGTGATTG |
| GATA2 | FWD | CAGCAAGGCTCGTTCCTGTT |
| | REV | GGCTTGATGAGTGGTCGGT |
| HHEX | FWD | TCAGAATCGACGCGCTAAATG |
| | REV | AGAGCTATCCAAAGAAGCACCT |
| KLRG1 | FWD | CCAGACCGCTGGATGAAATATG |
| | REV | CTGATTGTCCGTTATCACAAGGA |
| MMP16 | FWD | AGCACTGGAAGACGGTTGG |
| | REV | CTCCGTTCCGCAGACTGTA |
| OGN | FWD | TCTACACTTCTCCTGTTACTGCT |
| | REV | GAGGTAATGGTGTTATTGCCTCA |

IncuCyte Real-Time Imaging, Sulforhodamine B (SRB) and MU Assays

For proliferation studies, cells (5×10³) were seeded in 96-place wells and allowed to attach overnight, and then incubated in fresh growth medium in the presence of either the G9a inhibitor UNC0642 (Sigma Aldrich) or the vehicle control DMSO (Sigma Aldrich). Proliferation was evaluated via real-time imaging using IncuCyte Zoom (Essen BioScience) or by performing SRB assays at the end of the treatment. For SRB assays, medium was aspirated and cells were fixed in methylated spirits before being washed with water and stained with SRB (Sigma Aldrich) solution (0.4% in 1% acetic acid). The intensity of the staining was obtained using an optical plate reader at 564 nm. Cell viability was determined using a 3-(4,5-dimethylthiazol-2-yl)-2,5-diphenyltetrazolium bromide (MU) assay. Cells (3×10³/well) were seeded directly into 96-well plates and allowed to adhere overnight. 96 hours after drug treatment, 20 μL of MU (5 mg/mL; Sigma-Aldrich) was added. The plates were incubated at 37° C. for 3 hrs before the supernatants were removed, and 100 μL of isopropanol was added to each well. The absorbance value (optical density) of each well was measured at 570 nm.

Meta-Analysis of Breast Cancer Global Gene Expression

The genes identified from the microarray analysis (212 genes) were investigated in three large gene expression datasets from breast cancer; TCGA, ROCK and KM plotter datasets. The breast cancer cases in each of the three datasets (TCGA, ROCK and KM plotter) were allocated to one of four quartiles based on the hypoxia-G9a signature and the survival of these patients were compared. We also used the KM Plotter dataset (version 2014) to compare the relapse-free survival of breast cancer patients between tumors with the lowest expression (bottom 25%, quartile 1) to the rest of the tumors. Survival curves were constructed using GraphPad Prism v 6.0 (GraphPad Software, San Diego, Calif., USA), and the log-rank (Mantel-Cox) Test was used for statistical comparisons of survival curves.

### Example 5

### Gsa Inhibition Reduces Tumor Growth In Vivo

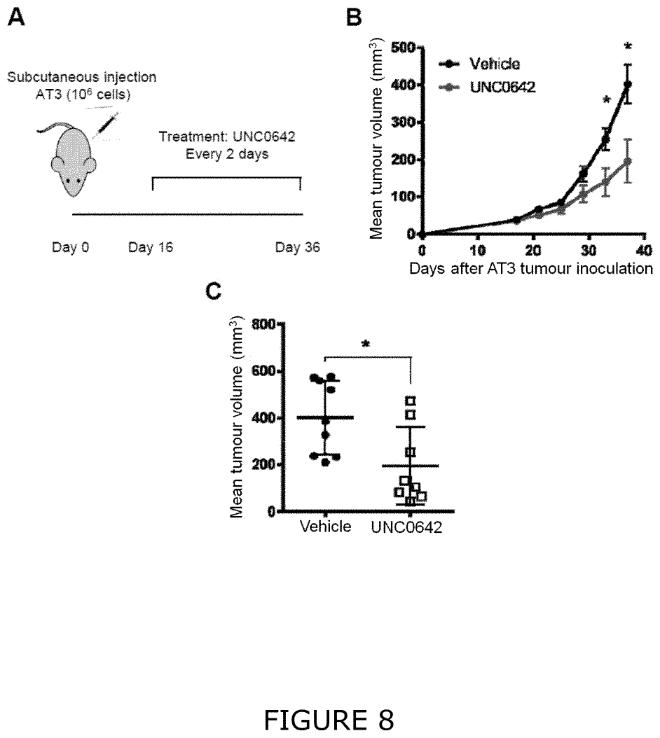

To determine the effect of inhibiting G9a methyltransferase activity on tumor growth, AT3 syngeneic mammary tumor cells were subcutaneously injected into C57BL/6 mice and allowed to form palpable tumors (over 2 weeks) before administering the UNC0642 G9a inhibitor (FIG. 8A). Consistent with the in vitro data, administration of UNC0642 significantly reduced tumor growth (FIG. 8B). The mean tumor volume was significantly lower in the UNC0642 treated group compared to that of the vehicle treated group at end-point (FIG. 8C) demonstrating that tumor growth can be suppressed by administering G9a inhibitor in vivo.

### Materials and Methods

C57BL/6 mice were purchased from the ARC Animal Resources Center. Groups of 8 to 10 mice per experiment were used for experimental tumor assays to ensure adequate power to detect biologic differences. All experiments were approved by the QIMR Berghofer Medical Research Institute Animal Ethics Committee. A total of 1×10⁶ AT3 tumor cells were subcutaneously injected into mice in a 100 μL volume (on day 0) and UNC0642 administered every two days between days 16 and day 36. Tumor growth was measured using a digital calliper, and tumor volumes are presented as mean±SEM.

### Example 6

### Gsa Signature as a Prognostic Indicator

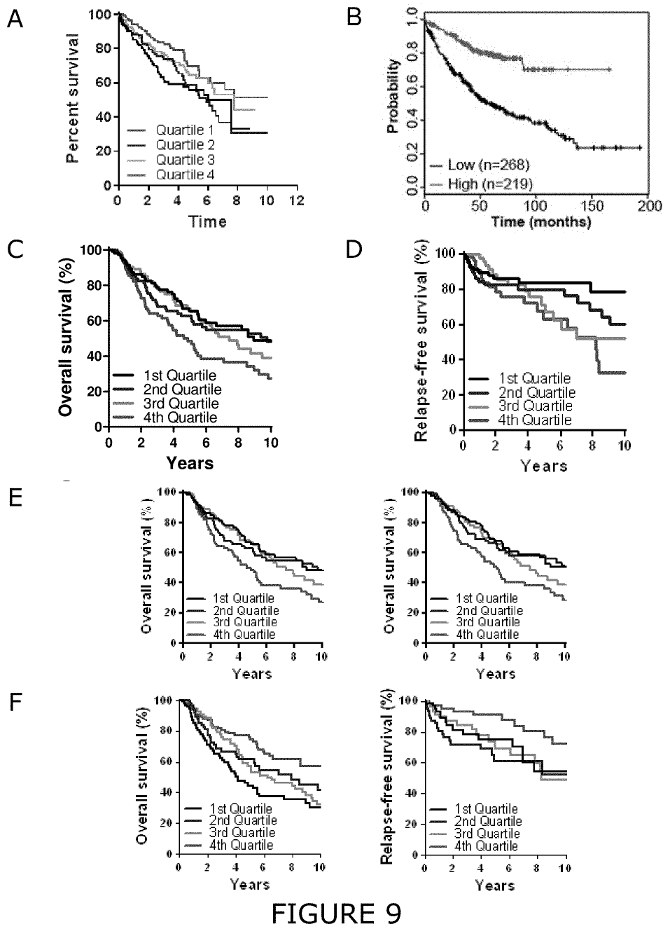

In order to demonstrate that the identified hypoxia markers are suitable for clinical diagnosis and/or prognosis, the biomarkers identified in the above breast cancer model were assessed in other hypoxic cancer types (i.e., kidney clear cell carcinoma and lung adenocarcinoma). FIG. 9A and FIG. 9B show patient survival curves in a retrospective meta-analysis of patient survival. Patients were stratified according to their average expression of the 10 above-identified hypoxia biomarkers.

Data from patients with kidney clear cell carcinoma (from the Cancer Genome Atlas (TCGA) was divided into quartiles (FIG. 9A) and the lung adenocarcinoma patient dataset from Kaplan Meyer Plotter was divided into two groups as dependent on whether they expressed “Low” or “High” levels of the hypoxia biomarkers. In both cancer-types, patients expressing high levels of the hypoxia biomarkers were demonstrated to be associated with a better survival chance when compared to those with lower expression levels of the hypoxia biomarkers.

Next, patients with melanoma were analyzed. FIG. 9C demonstrates that G9a expression is also associated with the survival outcome of melanoma and specifically, patients expressing a low amount of G9a protein are more likely to survive the disease than those expressing a high amount (FIG. 9C). Similarly, FIG. 9D shows that a low level of G9a protein correlates with an increased likelihood that the patient will remain relapse-free than patients with a high amount of G9a protein.

G9a expression also associates with survival outcome specifically in metastatic melanoma. For example, the overall survival of patients stratified using G9a expression was compared between all melanoma patients (471 patients; left hand graph) and metastatic patients (368 patients; right hand graph) and found that in both groups overall survival rates increased with those subjects with a low level of G9a protein.

Patients were allocated into one of four quartiles based on the average expression of a subset of hypoxic biomarkers (i.e., ARNTL, CD1C, HHEX, KLRG1 and MMP16). The subset of hypoxia biomarkers clearly associates with overall survival and relapse-free survival in melanoma. Patients with higher average expression (e.g., 4th Quartile) associated with a better overall survival (left-hand graph) and relapse-free survival (right-hand graph) as compared to those with lower expression (e.g., Quartile 1).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Harris A L (2002) Hypoxia—a key regulatory factor in tumor growth. Nat Rev Cancer 2(1): 38-47.
Semenza G L (2003) Targeting HIF-1 for cancer therapy. Nat Rev Cancer 3(10): 721-732.
Vaupel P (2004) Tumor microenvironmental physiology and its implications for radiation oncology. Semin Radiat Oncol 14(3): 198-206.
Bache M, Kappler M, Said H M, Staab A, & Vordermark D (2008) Detection and specific targeting of hypoxic regions within solid tumors: current preclinical and clinical strategies. Curr Med Chem 15(4): 322-338.
Erler J T, et al. (2004) Hypoxia-mediated down-regulation of Bid and Bax in tumors occurs via hypoxia-inducible factor 1-dependent and -independent mechanisms and contributes to drug resistance. Mol Cell Biol 24(7): 2875-2889.
Semenza G L (2000) Hypoxia, clonal selection, and the role of HIF-1 in tumor progression. Crit Rev Biochem Mol Biol 35(2): 71-103.
Hill R P, Marie-Egyptienne D T, & Hedley D W (2009) Cancer stem cells, hypoxia and metastasis. Semin Radiat Oncol 19(2): 106-111.
Pennacchietti 5, et al. (2003) Hypoxia promotes invasive growth by transcriptional activation of the met protooncogene. Cancer Cell 3(4): 347-361.
Chang Q, Jurisica I, Do T, & Hedley D W (2011) Hypoxia predicts aggressive growth and spontaneous metastasis formation from orthotopically grown primary xenografts of human pancreatic cancer. Cancer Res 71(8): 3110-3120.
McGary E C, Lev D C, & Bar-Eli M (2002) Cellular adhesion pathways and metastatic potential of human melanoma. Cancer Bio Ther 1(5): 459-465.
Lee H K, Lee D S, & Park J C (2015) Nuclear factor I-C regulates E-cadherin via control of KLF4 in breast cancer. BMC Cancer 15: 113.
Scully O J, Bay B H, Yip G, & Yu Y (2012) Breast cancer metastasis. Cancer Gen Prot proteomics 9(5): 311-320.
Ema M, et al. (1997) A novel bHLH-PAS factor with close sequence similarity to hypoxia-inducible factor lalpha regulates the VEGF expression and is potentially involved in lung and vascular development. Proc Natl Acad Sci USA 94(9): 4273-4278.
Semenza G L & Wang G L (1992) A nuclear factor induced by hypoxia via de novo protein synthesis binds to the human erythropoietin gene enhancer at a site required for transcriptional activation. Molecular and cellular biology 12(12): 5447-5454.
Dewhirst M W, Cao Y, & Moeller B (2008) Cycling hypoxia and free radicals regulate angiogenesis and radiotherapy response. Nat Rev Cancer 8(6): 425-437.
Poon E, Harris A L, & Ashcroft M (2009) Targeting the hypoxia-inducible factor (HIF) pathway in cancer. Expert Rev Mol Med 11: e26.
Ivan M, et al. (2001) HlFalpha targeted for VHL-mediated destruction by proline hydroxylation: implications for O₂ sensing. Science 292(5516): 464-468.
Jaakkola P, et al. (2001) Targeting of HIF-alpha to the von Hippel-Lindau ubiquitylation complex by O₂-regulated prolyl hydroxylation. Science 292(5516): 468-472.
Maxwell P H, et al. (1999) The tumor suppressor protein VHL targets hypoxia-inducible factors for oxygen-dependent proteolysis. Nature 399(6733): 271-275.
Ohh M, et al. (2000) Ubiquitination of hypoxia-inducible factor requires direct binding to the beta-domain of the von Hippel-Lindau protein. Nat Cell Biol 2(7): 423-427.
Yun Z, Maecker H L, Johnson R S, & Giaccia A J (2002) Inhibition of PPAR gamma 2 gene expression by the HIF-1-regulated gene DEC1/Stra13: a mechanism for regulation of adipogenesis by hypoxia. Dev Cell 2(3): 331-341.
Chakrabarti J, et al. (2004) The transcription factor DEC1 (stral3, SHARP2) is associated with the hypoxic response and high tumor grade in human breast cancers. Br 3 Cancer 91(5): 954-958.
Ivanov S V, Salnikow K, Ivanova A V, Bai L, & Lerman M I (2007) Hypoxic repression of STAT1 and its downstream genes by a pVHL/HIF-1 target DEC1/STRA13. Oncogene 26(6): 802-812.
Tachibana M, et al. (2005) Histone methyltransferases G9a and GLP form heteromeric complexes and are both crucial for methylation of euchromatin at H3-K9. Genes Dev 19(7): 815-826.
Chen M W, et al. (2010) H3K9 histone methyltransferase G9a promotes lung cancer invasion and metastasis by silencing the cell adhesion molecule Ep-CAM. Cancer Res 70(20): 7830-7840.
Dong C, et al. (2012) G9a interacts with Snail and is critical for Snail-mediated E-cadherin repression in human breast cancer. J Clin Invest 122(4): 1469-1486.
Liu 5, et al. (2015) G9a is essential for EMT-mediated metastasis and maintenance of cancer stem cell-like characters in head and neck squamous cell carcinoma. Oncotarget 6(9): 6887-6901.
Zhong X, et al. (2015) Overexpression of G9a and MCM7 in oesophageal squamous cell carcinoma is associated with poor prognosis. Histopathology 66(2): 192-200.
Hua K T, et al. (2014) The H3K9 methyltransferase G9a is a marker of aggressive ovarian cancer that promotes peritoneal metastasis. Mol Cancer 13: 189.
Wozniak R J, Klimecki W T, Lau S S, Feinstein Y, & Futscher B W (2007) 5-Aza-2′-deoxycytidine-mediated reductions in G9A histone methyltransferase and histone H3 K9 di-methylation levels are linked to tumor suppressor gene reactivation. Oncogene 26(1): 77-90.
Chen H, Yan Y, Davidson T L, Shinkai Y, & Costa M (2006) Hypoxic stress induces dimethylated histone H3 lysine 9 through histone methyltransferase G9a in mammalian cells. Cancer Res 66(18): 9009-9016.
Hwang D, et al. (2005) A data integration methodology for systems biology: experimental verification. Proc Natl Acad Sci USA 102(48): 17302-17307.
Yamamizu K, et al. (2012) Protein kinase A determines timing of early differentiation through epigenetic regulation with G9a. Cell Stem Cell 10(6): 759-770.
Joyce J A & Pollard J W (2009) Microenvironmental regulation of metastasis. Nature reviews. Cancer 9(4): 239-252.
Hu Z, et al. (2009) A compact VEGF signature associated with distant metastases and poor outcomes. BMC medicine 7: 9.
Li L, et al. (2014) Quantitative proteomics approach to screening of potential diagnostic and therapeutic targets for laryngeal carcinoma. PloS One 9(2):e90181.
Hu Q P, Kuang J Y, Yang Q K, Bian X W, & Yu S C (2016) Beyond a tumor suppressor: Soluble E-cadherin promotes the progression of cancer. IntJ Cancer 138(12): 2804-2812.
Li Y W, et al. (2014) Decreased expression of GATA2 promoted proliferation, migration and invasion of HepG2 in vitro and correlated with poor prognosis of hepatocellular carcinoma. PloS One 9(1): e87505.
Noy P, Williams H, Sawasdichai A, Gaston K, & Jayaraman P S (2010) PRH/Hhex controls cell survival through coordinate transcriptional regulation of vascular endothelial growth factor signaling. Mol Cell Biol 30(9): 2120-2134.
Kershaw R M, Siddiqui Y H, Roberts D, Jayaraman P S, & Gaston K (2014) PRH/HHex inhibits the migration of breast and prostate epithelial cells through direct transcriptional regulation of Endoglin. Oncogene 33(49): 5592-5600.
Scholzel 5, et al. (2000) Carcinoembryonic antigen family members CEACAM6 and CEACAM7 are differentially expressed in normal tissues and oppositely deregulated in hyperplastic colorectal polyps and early adenomas. Am J Path 156(2): 595-605.
Messick C A, et al. (2010) CEACAM-7: a predictive marker for rectal cancer recurrence. Surgery 147(5): 713-719.
Yeh C M, et al. (2014) Epigenetic silencing of ARNTL, a circadian gene and potential tumor suppressor in ovarian cancer. Int J Oncology 45(5): 2101-2107.
Tachibana M, Matsumura Y, Fukuda M, Kimura H, & Shinkai Y (2008) G9a/GLP complexes independently mediate H3K9 and DNA methylation to silence transcription. EMBO J 27(20): 2681-2690.
Ma D K, Chiang C H, Ponnusamy K, Ming G L, & Song H (2008) G9a and Jhdm2a regulate embryonic stem cell fusion-induced reprogramming of adult neural stem cells. Stem Cells 26(8): 2131-2141.
Mole D R, et al. (2009) Genome-wide association of hypoxia-inducible factor (HIF)-lalpha and HIF-2alpha DNA binding with expression profiling of hypoxia-inducible transcripts. J Biol Chem 284(25): 16767-16775.
Pugh C W, Ratcliffe P J (2003) Regulation of angiogenesis by hypoxia: role of the HIF system. Nat Med 9(6): 677-684.
Lee J S, et al. (2010) Negative regulation of hypoxic responses via induced Reptin methylation. Mol Cell 39(1): 71-85.
Lee J S, et al. (2011) Hypoxia-induced methylation of a pontin chromatin remodeling factor. Proc Natl Acad Sci USA 108(33): 13510-13515.
Baselga J, et al. (2012) Everolimus in postmenopausal hormone-receptor-positive advanced breast cancer. New Eng J Med 366(6): 520-529.
Figueroa-Magalhaes M C, Jelovac D, Connolly R M, & Wolff A C (2014) Treatment of HER2-positive breast cancer. Breast 23(2): 128-136.
Ngiow S F, et al. (2012) Anti-TIM3 antibody promotes T cell IFN-gamma-mediated antitumor immunity and suppresses established tumors. Cancer research 71(10): 3540-3551.
   GATA2, CEACAM7, OGN, and AGTR1; (2) determining an indicator using the biomarker value(s); and (3) administering an effective amount of a G9a antagonist to the subject on the basis that the indicator is at least partially indicative of the likelihood of the presence of the hypoxic condition in the subject.

6. A method of reducing the malignancy of a hypoxic tumor in a subject, the method comprising, consisting, or consisting essentially of: (1) determining a biomarker value that is measured or derived for at least one hypoxia biomarker (e.g., 1, 2, 3, 4, 5, 6, 7, 8, 9, or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; (2) determining an indicator using the biomarker value(s); and (3) administering an effective amount of a G9a antagonist to the subject on the basis that the indicator is at least partially indicative of the likelihood that the tumor is hypoxic.

7. A method of treating a hypoxic tumor in a subject, the method comprising, consisting, or consisting essentially of: (1) determining a biomarker value that is measured or derived for at least one hypoxia biomarker (e.g., 1, 2, 3, 4, 5, 6, 7, 8, 9, or 10 hypoxia biomarkers) in sample obtained from the subject, wherein the at least one hypoxia biomarker is selected from ARNTL, CD1C, HHEX, KLRG1, MMP16, FGFR2, GATA2, CEACAM7, OGN, and AGTR1; and (2) determining an indicator using the biomarker value(s); and (3) administering an effective amount of a G9a antagonist to the subject on the basis that the indicator is at least partially indicative of the likelihood that the tumor is hypoxic.

8. The method of any one of claims 5 to 7, wherein the subject is administered with an ancillary treatment.

9. The method of claim 8, wherein the ancillary treatment is chemotherapy and/or radiotherapy.

10. The method of any one of claims 1 to 9, wherein the indicator comprises a biomarker value for at least 2, 3, 4, 5, 6, 7, 8, 9, 10, 15, 20 (and every integer in between) hypoxia biomarkers.

11. The method of any one of claims 1 to 10, wherein the sample is a biological sample.

12. The method of claim 11, wherein the biological sample comprises tumor cells.

13. The method of claim any one of claims 1 to 12, wherein the at least one hypoxia biomarker is selected from the group consisting of: (a) a polynucleotide expression product comprising a nucleotide sequence that shares at least 70% (or at least 71% to at least 99% and all integer percentages in between) sequence identity with the sequence set forth in any one of SEQ ID NO: 1-10, or a complement thereof; (b) a polynucleotide expression product comprising a nucleotide sequence that encodes a polypeptide comprising the amino acid sequence set forth in any one of SEQ ID NO: 202-211; (c) a polynucleotide expression product comprising a nucleotide sequence that encodes a polypeptide that shares at least 70% (or at least 71% to at least 99% and all integer percentages in between) sequence similarity or identity with at least a portion of the sequence set forth in SEQ ID NO: 202-211; (d) a polynucleotide expression product comprising a nucleotide sequence that hybridizes to the sequence of (a), (b), (c) or a complement thereof, under medium or high stringency conditions; (e) a polypeptide expression product comprising the amino acid sequence set forth in any one of SEQ ID NO: 202-211; and (f) a polypeptide expression product comprising an amino acid sequence that shares at least 70% (or at least 71% to at least 99% and all integer percentages in between) sequence similarity or identity with the sequence set forth in any one of SEQ ID NO: 202-211.

14. The method of any one of claims 1 to 13, wherein the biomarker value is at least partially indicative of a concentration of the at least one hypoxia biomarker in the sample obtained from the subject.

15. The method of any one of claims 1 to 13, wherein the biomarker value is at least partially indicated of the level of gene expression of the at least one hypoxia biomarker in the sample obtained from the subject.

16. The method of any one of claims 1 to 13, wherein the biomarker value includes the abundance of the biomarker.

17. The method of any one of claims 1 to 16 wherein the level of the at least one hypoxia biomarker is reduced relative to the level of the biomarker that correlates with the presence of normal (i.e., non-hypoxic) conditions, and the indicator is thereby determined to be at least partially indicative of a hypoxia.

18. A method according to any one of claims 1 to 16, wherein the level of the at least one hypoxia biomarker is about the same as the level of the biomarker that correlates with the presence of normal (i.e., non-hypoxic) conditions, and the indicator is determined to be at least partially indicative of a normoxia

19. A method of determining an indicator used in assessing a likelihood of the presence or absence of a hypoxic condition (e.g., a hypoxic cancer) in a subject, the method comprising, consisting or consisting essentially of: (1) determining a biomarker value that is measured or derived for at least one group 1 hypoxia biomarker (e.g., 1, 2, 3, 4, 5, 6, 7, 8, 9 or 10 hypoxia biomarkers) in a sample obtained from the subject, wherein the at least one hypoxia biomarker is selected from ARNTL, CD1C, HHEX, KLRG1, MMP16, FGFR2, GATA2, CEACAM7, OGN, and AGTR1; (2) determining a biomarker value that is measured or derived for a group 2 hypoxia biomarker, wherein the group 2 hypoxia biomarker is G9a; and (3) determining the indicator using the biomarker values, wherein the indicator is at least partially indicative of the likelihood of the presence or absence of the hypoxic condition in the subject.

20. The method of claim 19, wherein the method further comprises applying a combining function to the at least one group 1 hypoxia biomarker value(s) and the group 2 hypoxia biomarker

21. A method according to claim 19 or claim 20, wherein the indicator is a ratio of the biomarker values recorded on the group 1 hypoxia biomarker and the group 2 hypoxia biomarkers.

22. The method of any one of claims 1 to 21, wherein the biomarker value(s) is(are) measured using microscopy, flow cytometry, immunoassays, mass spectrometry, sequencing platforms, array and hybridization platforms, or a combination thereof.

23. A composition for determining an indicator used in assessing a likelihood of hypoxia, the composition comprising, consisting, or consisting essentially of at least one cDNA and at least one oligonucleotide primer or probe that hybridizes to the cDNA, wherein the at least one cDNA is a selected from ARNTL, CD1C, HHEX, KLRG1, MMP16, FGFR2, GATA2, CEACAM7, OGN, and AGTR1.

24. A complex comprising, consisting, or consisting essentially of at least one cDNA and at least one oligonucleotide primer or probe that hybridizes to the cDNA, wherein the at least one cDNA is a selected from ARNTL, CD1C, HHEX, KLRG1, MMP16, FGFR2, GATA2, CEACAM7, OGN, and AGTR1.

25. The composition of claim 23 or the complex of claim 24, comprising two or more cDNAs and at least one oligonucleotide primer or probe that hybridizes to an individual one of the cDNAs.

26. The composition or complex of claim 25, wherein the composition or complex comprises a population of cDNAs corresponding to mRNA derived from a cell or cell population.

27. The composition or complex of to claim 26, wherein the cell is a cell of a tumor.

28. The composition or complex of any one of claims 23 to 27, wherein the at least one oligonucleotide primer or probe is hybridized to an individual one of the cDNAs.

29. The composition or complex of any one of claims 23 to 28, wherein the composition or complex further comprises a labelled reagent for detecting the cDNAs.

30. The composition or complex according to claim 29, wherein the labelled reagent is a labelled said at least one oligonucleotide primer or probe.

31. The composition or complex of claim 30, wherein the labelled reagent is a labelled said cDNA.

32. A composition or complex of any one of claims 23 to 31, wherein the at least one oligonucleotide primer or probe is in a form other than a high density array.

33. A kit for determining an indicator indicative of the likelihood of hypoxia in a subject, the kit comprising, consisting, or consisting essentially of, (a) at least one reagent that allows quantification of a hypoxia biomarker, wherein the at least one hypoxia biomarker is selected from ARNTL, CD1C, HHEX, KLRG1, MMP16, FGFR2, GATA2, CEACAM7, OGN, and AGTR1; and optionally (b) instructions for using the at least one reagent.

34. A composition comprising, consisting, or consisting essentially of at least one (i.e., 2, 3, 4, 5, 6, 7, 8, 9, 10, or more than 10) cDNAs, and for each respective cDNA two oligonucleotide primers that hybridize to opposite complementary strands of the cDNA, and an oligonucleotide probe that hybridizes to the cDNA, wherein the at least one cDNA is a selected from ARNTL, CD1C, HHEX, KLRG1, MMP16, FGFR2, GATA2, CEACAM7, OGN, and AGTR1.

35. The composition of claim 34, wherein the composition further comprises a labelled reagent for detecting the cDNA.

36. The composition of claim 34, wherein the labelled reagent is a labelled said at least one oligonucleotide primer or probe.
